(19) United States
(12) Patent Application Publication
(10) Pub. No.: US 2025/0057964 A1
(43) Pub. Date: Feb. 20, 2025

(54) LIGAND-DRUG-CONJUGATE COMPRISING A SINGLE MOLECULAR WEIGHT POLYSARCOSINE

(71) Applicant: Mablink Bioscience SAS, Lyon (FR)

(72) Inventor: Warren VIRICEL, Lyon (FR)

(21) Appl. No.: 18/894,701

(22) Filed: Sep. 24, 2024

Related U.S. Application Data
(63) Continuation of application No. 16/758,638, filed on Apr. 23, 2020, now abandoned.

(30) Foreign Application Priority Data
Oct. 23, 2017 (EP) 17306457.7
Jul. 18, 2018 (EP) 18305985.6

(51) Int. Cl. A61K 47/62; A61K 47/68

(52) U.S. Cl. CPC A61K 47/62; A61K 47/6803; A61K 47/68031; A61K 47/68037; A61K 47/6855; A61K 47/6883

(57) ABSTRACT

The invention relates to a Ligand-Drug-Conjugate (LDC) comprising a single molecular weight homopolymer, in particular a single molecular weight polysarcosine.

The present invention also relates to a Ligand-Drug-Conjugate (LDC) comprising a single molecular weight homopolymer, in particular a single molecular weight polysarcosine.

Ligand-drug-conjugates (LDC) are comprised of at least one ligand unit which is a polypeptide or protein that is covalently linked to at least one therapeutic, diagnostic or labelling molecule (hereinafter referred as drug or D) via a synthetic linker. This synthetic linker may comprise one or several divalent arms for joining the ligand unit(s) and the drug unit(s), which may be selected from spacers, connectors and cleavable moieties. Said linker may also bear any monovalent moiety that can improve the LDC performance, such as storage stability, plasmatic stability or pharmacokinetics properties. The protein or polypeptide is usually a targeting unit, but can have intrinsic therapeutic properties. When the ligand unit of the conjugate is an antibody or an antibody fragment and is associated with a cytotoxic or chemotherapy drug, the term antibody-drug-conjugates (ADCs) is commonly used.

The design of an ADC involves consideration of numerous diverse factors: (i) the nature, the number, the overall hydrophobicity and the location of the synthetic linker used for conjugation on the ligand; (ii) the nature and mechanism of action of the drug; (iii) the structural elements responsible for the drug release after cell internalization and during intracellular trafficking; (iv) the properties of the monoclonal antibody (mAb) and the selected antigen target. Recent methodologies have addressed some of the shortcomings of available ADCs, such as heterogeneous drug loading (ADC subspecies with different pharmacological properties), limited mAb-linker or drug-linker stability, and suboptimal pharmacokinetic properties (Beck et al., Nat. Rev. Drug. Discov., 2017, 16(5), 315-337).

Another important factor to consider when designing conjugates is the drug ratio (or drug-antibody-ratio (DAR) for ADCs), which is the average number of drug units conjugated to the antibody. As a result of recent findings, the actual trend in the ADC field is to generate and bring to the clinic homogeneous conjugates with low-to-moderate DAR (usually 2 to 4). Nonetheless more recently have emerged new linker-drug technologies aiming to overcome the drawbacks (unfavorable pharmacokinetic properties and tendency to form aggregates thus complicating conjugate formulation) of highly loaded ADC's. Such technologies have the potential to bring to the clinic next-generation ADC's with improved efficacy, improved pharmacokinetics properties, improved therapeutic indices and able to target tumors with low target expression, slow internalization or inefficient intracellular processing. To achieve such high payload loadings without sacrificing pharmacokinetic properties and formulation stability, new linker-drug design approaches aiming to mask the apparent hydrophobicity of cytotoxic payloads needs to be developed.

In W02014/093394A1, it is reported a protein-polymer-drug conjugate that exhibits high drug load and strong binding to target antigen. This conjugate involves a biodegradable and biocompatible poly-[1-hydroxymethylethylene hydroxylmethylformal] polymeric entity, which allows the conjugation of approximately 12 to 25 cytotoxic molecules per mAb with good pharmacokinetic properties. The main drawback of this approach is the extreme polydispersity of the final conjugates, arising from (i) the polydisperse nature of the linker, (ii) the heterogeneous number of cytotoxic molecules per polymeric arm and (iii) the heterogeneous number of polymeric arm grafted per mAb.

In W02015/057699A2 and W02016/059377A1, it is reported the formulation of 8 to 36-drug loaded ADC's by inclusion of orthogonal poly-ethyleneglycol (PEG) moieties in the linker design. PEG is well-known to improve hydrophilicity, stability and circulation time of small drugs, proteins, bioconjugates and nanoparticles due to its hydrophilic properties, biocompatibility and high hydration shell. However, PEG is not exempt of drawbacks, such as non-biodegradability, possible complement activation leading to hypersensitivity and unclear pharmacokinetics because of anti-PEG antibodies expressed by some healthy individuals.

There is a need for ligand-drug-conjugates that combines: (i) high drug loading while maintaining favorable pharmacokinetic and stability properties, (ii) complete homogeneity of the conjugate at the drug-linker level (chemically monodisperse drug-linker) and at the conjugate level (homogeneously-loaded conjugate) and, (iii) based on a biodegradable hydrophilic homopolymer that acts as a hydrophobicity masking moiety.

Polysarcosine (poly-N-methylglycine or PSAR) could be an alternative to PEG and could be used to design novel protein conjugates with improved properties. PSAR is a highly hydrophilic, biodegradable, non-immunogenic and water-soluble polymer that has been employed in several delivery systems for drugs or diagnostics. To date, PSAR is only available as a polydisperse form, as it is accessed via a condensative ring-opening polymerization reaction of sarcosine N-carboxyanhydride (NCA) or sarcosine N-thiocarboxyanhydride (NTA). Albeit relatively well defined with acceptable dispersities (Gaussian distribution of molecular weights with a polydispersity index >1), these polydisperse PSAR cannot be used in certain application areas that require the use of shorter homopolymer compounds having consistent length (unique and specific molecular weight) and therefore absolute homogeneity.

Using discrete monodisperse PSAR for macromolecule modification is a requirement to develop conjugates with absolute chemical homogeneity. Such homogeneous conjugates have the advantage of sharing the exact same pharmacological properties (pharmacokinetic and potency), are more straightforward to characterize, allow greater control of the reproducibility of the manufacturing process and meets the requirements of the more and more stringent regulatory requirements for bioconjugates.

In accordance with the present invention, it has been obtained discrete monodisperse PSAR homopolymers with defined chain length using a step-wise on-resin sub-monomer approach. This method is inexpensive, easily scalable and gives final products with acceptable yields and excellent monomeric purity. These monodisperse PSAR homopolymers were used in protein conjugation technologies to provide Ligand-drug-conjugates (LDC) with improved drug loadability, pharmacokinetics and therapeutic efficacy.

Thus, the present invention provides a single molecular weight monofunctional homopolymer which fulfills the requirements above to be used in conjugation technologies, and specifically in LDC.

This homopolymer has formula (I) below

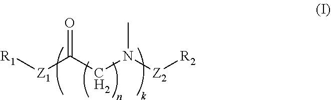

Wherein
R₁ and R₂ are different, and
one of R₁ and R₂ is H or an inert group, the other one of R₁ and R₂ being a functionalized reactive group, said group being reactive for covalently binding a bindable group, in such reaction conditions that the inert group is non-reactive,
Z₁ and Z₂, identical or different, are optional spacers, and
n is 1 or more and k is 2 or more.

Before exposing the invention in detail, definitions of terms employed in the present text are given below.

### Definitions

In accordance with the invention, any compound such as reactant, product, monomer, homopolymer, unit, may be in the form of salts, including acid addition salts, base addition salts, metal salts and ammonium and alkylated ammonium salts. Such salts are well-known from the skilled in the art. In view of the intended uses of an homopolymer of the invention, they are preferably in the form of pharmaceutically acceptable salts.

A single molecular weight homopolymer refers to a homopolymer having a unique and specific, molecular weight, as opposed to a mixture of homopolymers of the same nature but having a distribution of sizes and molecular weights, centered on an average molecular weight. A single molecular weight homopolymer can be defined with one absolute molecular formula having an absolute number of atoms.

The single molecular weight homopolymer can also be referred as “monodisperse” with a polydispersity index (PDI) equal to 1, as opposed to polydisperse homopolymer traditionally obtained by one-pot polymerization processes and having a PDI>1. It is generally admitted in the present description that the terms “monodisperse” and “discrete” are interchangeable, both defining a homopolymer having a unique and absolute molecular weight, molecular formula and molecular architecture, despite the fact that the term “monodisperse” does not accurately reflects the fabrication procedure of the product.

An inert group or capping group refers to any chemical non-reactive group that terminates one end of the homopolymer, said group being non-reactive when compared with the functionalized reactive group that terminates the other end of the homopolymer, in determined reaction conditions. The resulting homopolymer is in a way end-capped by this inert group and is not intended to be covalently bonded, when it is used, in particular in LDC technologies. In an embodiment, the group may only be rendered inert after its covalent binding to one end of the homopolymer.

Non-exhaustive listing of inert groups includes: acyl group especially acetyl group, amide group, alkyl group especially a C₁₋₂₀ alkyl group, alkyl ether group, alkyl ester group, alkyl orthoester group, alkenyl group, alkynyl group, aryl group, aryl ester group, tertiary amine group, hydroxyl group, aldehyde group. Said inert group may also be selected from the same listing of groups that defines a functionalized reactive group (see definition of a functionalized reactive group below).

A functionalized reactive group refers to any chemical moiety that is being reactive for covalently binding a bindable group, said group being reactive when compared with the inert group, in determined reaction conditions. In particular, it may bind the following groups: carboxylic acid; primary amine; secondary amine; tertiary amine; hydroxyl; halogen; activated ester such as N-hydroxysuccinimide ester, perfluorinated esters, nitrophenyl esters, aza-benzotriazole and benzotriazole activated ester, acylureas; alkynyl; alkenyl; azide; isocyanate; isothiocyanate; aldehyde; thiol-reactive moieties such as maleimide, halomaleimides, haloacetyls, pyridyl disulfides; thiol; acrylate; mesylate; tosylate; triflate, hydroxylamine; chlorosulfonyl; boronic acid —B(OR′)₂ derivatives wherein R′ is hydrogen or alkyl group.

Non-exhaustive listing of functionalized reactive group includes: carboxylic acid; primary amine; secondary amine; tertiary amine; hydroxyl; halogen; activated ester such as N-hydroxysuccinimide ester, perfluorinated esters, nitrophenyl esters, aza-benzotriazole and benzotriazole activated ester, acylureas; alkynyl; alkenyl; azide; isocyanate; isothiocyanate; aldehyde; thiol-reactive moieties such as maleimide, halomaleimides, haloacetyls, pyridyl disulfides; thiol; acrylate; mesylate; tosylate; triflate, hydroxylamine; chlorosulfonyl; boronic acid —B(OR′)₂ derivatives wherein R′ is hydrogen or alkyl group.

It should be mentioned that the terms “inert” and “functionalized reactive” for an inert group and a functionalized reactive group, respectively, are interdependent. This means that, in determined reaction conditions of a homopolymer of the invention as defined in any one of formulae (I), (II) and (III), the inert group will not react and the functionalized reactive group will react to covalently bind a reactant. Said inert group and functionalized reactive group in a homopolymer of any one of formulae (I), (II) and (III) are therefore different, but they may globally be selected from the same listing of groups.

The term “group” in a functionalized reactive group or an inert group in accordance with the present invention should be understood as a group which doesn't exhibit any other function than being able to covalently bind a reactant or being inert, respectively, in determined reaction conditions.

Alkyl, used alone or as part of alkyl ether or alkyl ester for example, refers to a saturated, straight-chained or branched hydrocarbon group having 1-20 carbon atoms, preferably 1-12, more preferably 1-6, especially 1-4.

Alkenyl and alkynyl refer to at least partially unsaturated, straight-chained or branched hydrocarbon group having 2-20 carbon atoms, preferably 2-12, more preferably 2-6, especially 2-4.

Aryl, used alone or as part of aryl ester for example, refers to an aromatic group which has one ring or more, containing from 6-14 ring carbon atoms, preferably 6-10, especially 6.

Alkylene, used alone or as part of alkylene glycol for example, refers to a divalent saturated, straight-chained or branched hydrocarbon group having 1-20 carbon atoms, preferably 1-12, more preferably 1-6, especially 1-4.

Arylene refers to a divalent aryl group as defined above.

Heteroalkyl refers to a straight or branched hydrocarbon chain consisting of 1 to 20 or 1 to 10 carbon atoms and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule.

Heteroalkylene refers to a divalent heteroalkyl as defined above. For heteroalkylene groups, heteroatoms can also occupy either or both of the chain termini.

C₃-C₈ carbocycle refers to a 3-, 4-, 5-, 6-, 7- or 8-membered monovalent, substituted or unsubstituted, saturated or unsaturated non-aromatic monocyclic or bicyclic carbocyclic ring.

C₃-C₈ carbocyclo refers to a divalent C₃-C₈ carbocycle as defined above.

C₃-C₈ heterocycle refers to a monovalent substituted or unsubstituted aromatic or non-aromatic monocyclic or bicyclic ring system having from 3 to 8 carbon atoms (also referred to as ring members) and one to four heteroatom ring members independently selected from N, O, P or S. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure.

C₃-C₈ heterocyclo refers to a divalent C₃-C₈ heterocycle as defined above.

Furthermore, the terms alkyl, alkenyl, alkynyl, aryl, alkylene, arylene, heteroalkyl, heteroalkylene, C₃-C₈ carbocycle, C₃-C₈ carbocyclo, C₃-C₈ heterocycle, C₃-C₈ heterocyclo refer to optionally substituted groups with one or more of the substituents selected from: —X, —R′, —O⁻, —OR′, ═O, —SR′, —S—, —NR′₂, —NR′₃, ═NR′, —CX₃, —CN, —OCN, —SCN, —N═C═O, —NCS, —NO, —NO₂, ═N₂, —N₃, —NRC(═O)R′, —C(═O)R′, —C(═O)NR′₂, —SO₃—, —SO₃H, —S(═O)₂R′, —OS(═O)₂OR′, —S(═O)₂NR′, —S(═O)R′, —OP(═O)(OR′)₂, —P(═O)(OR′)₂, —PO₃—, —PO₃H₂, —C(═O)R′, —C(═O)X, —C(═S)R′, —CO₂R′, —CO₂, —C(═S)OR′, C(═O)SR′, C(═S)SR′, C(═O)NR′₂, C(═S)NR′₂, and C(═NR′)NR′₂, where each X is independently a halogen: —F, —CI, —Br, or —I; and each R′ is independently —H, —C₁C₂₀ alkyl, —C₆-C₂₀ aryl, or —C₃-C₁₄ heterocycle.

Acyl group refers to —CO-alkyl wherein alkyl has the definition above.

A monofunctional homopolymer comprises a single type of monomer (e.g. N-methylglycine monomer for polysarcosine) having one ending bearing a functionalized reactive group as defined above and another ending bearing H or an inert group as defined above.

Support for solid-phase peptide synthesis (SPPS) refers to a support which is usually employed in SPPS, a well-known process in which a peptide anchored to a support, an insoluble polymer, is assembled by the successive addition of Fmoc- or Boc- protected aminoacids, via repeated cycles of deprotection-wash-coupling-wash. Each aminoacid addition is referred to as a cycle of: (i) cleavage of the Na-protecting group, (ii) washing steps, (iii) coupling of a fluroenylmethoxycarbonyl- (Fmoc-) or tert-butyloxycarbonyl- (Boc-) protected aminoacid using coupling reagents and a non-nucleophilic base, (iv) washing steps. As the growing chain is bound to said support the excess of reagents and soluble by-products can be removed by simple filtration. Because repeated coupling reactions with hindered Fmoc- or Boc-protected N-methylated aminoacids are difficult and often suboptimal, low crude purity, difficult purification and low yields are to be expected with this technique. Examples of said support are Wang resin, Rink amide resin, trityl- and 2-chlorotrityl resins, PAM resin, PAL resin, Sieber amide resin, MBHA resin, HMPB resin, HMBA resin which are commercially available and on which the peptide is directly or indirectly bound.

The term orthogonal connector refers to a branched linker unit component that connects a ligand to a homopolymer unit and to a drug unit so that the homopolymer unit is in a parallel configuration (as opposed to a series configuration) in relation to the drug unit. The orthogonal connector is a scaffold bearing attachment sites for components of the ligand-drug-conjugate, namely the ligand, the homopolymer and the drug units. The term “parallel” is used to denote branching of two components of a ligand-drug-conjugate (LDC) but is not being used to denote that the two components are necessarily in close proximity in space or have the same distance between them.

An exemplary graphical representation of a LDC having a homopolymer (e.g. polysarcosine) unit that is in a parallel (i.e. branched) orientation in relation to the drug unit is as follows:

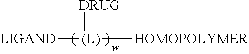

wherein (L) is the orthogonal connector unit and w is 1 or more, typically from 1 to 5, preferably 1 to 4, more preferably 1 to 3 and even 1 and 2. This orthogonal architecture is not to be confused with a linear architecture. An exemplary graphical representation of a LDC having a homopolymer (e.g. polysarcosine) unit that is in a serial (i.e. linear) orientation in relation to the drug unit is as follows:

LIGAND-HOMOPOLYMER-DRUG

Non-exhaustive listing of orthogonal connectors includes: natural or non-natural aminoacids, for example lysine, glutamic acid, aspartic acid, serine, tyrosine, cysteine, selenocysteine, glycine, homoalanine; amino alcohols; amino aldehydes; polyamines or any combination thereof. From his knowledge, the one skilled in the art is capable to select an orthogonal connector which is appropriate to the expected LDC compound. Advantageously, L is one or more natural or non-natural aminoacids. In one embodiment, L is selected from glutamic acid, lysine and glycine.

A spacer is a divalent linear arm that covalently binds two components of the ligand-drug-conjugate, such as:
the ligand unit and the orthogonal connector unit,
the orthogonal connector unit and an homopolymer unit,
the orthogonal connector and the cleavable moiety,
the cleavable moiety and the drug, or
the orthogonal connector and the drug.

For example, a spacer is a divalent linear alkylene group, preferably (CH₂)₄. Non-exhaustive listing of spacer units includes: alkylene, heteroalkylene (so an alkylene interrupted by at least one heteroatom selected from Si, N, O and S); alkoxy; polyether such as polyalkylene glycol and typically polyethylene glycol; one or more natural or non-natural aminoacids such as glycine, alanine, proline, valine, N-methylglycine; C₃-C₈ heterocyclo; C₃-C₈ carbocyclo; arylene, and any combination thereof. The spacer, when present between the cleavable moiety and the drug unit or between the orthogonal connector and the drug unit, can be linked to one or more drug units. For example, the spacer can be linked to 1 to 4 drug units, preferably 1 to 2 drug units. In one embodiment, the spacer between the cleavable moiety and the drug units is (4-amino-1,3-phenylene)dimethanol.

In one embodiment, the spacer unit is of formula (XVII), (XVIII), (XIX), (XX), (XXI) or (XXII),

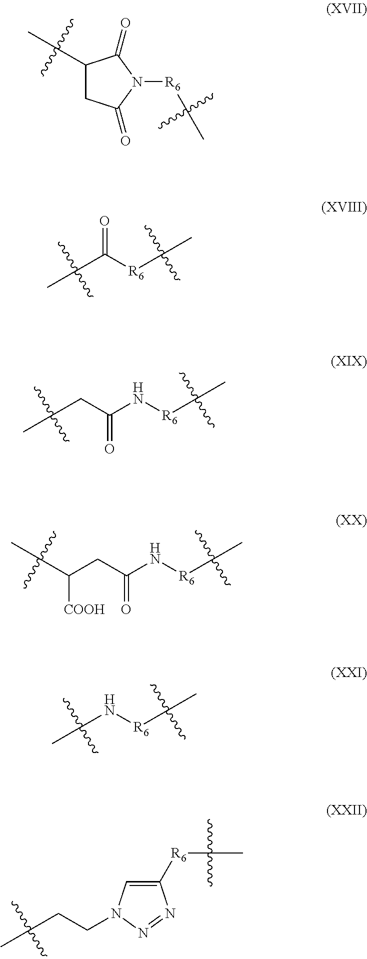

wherein the wavy bonds represent the attachment points and R₆ is —C₁-C₁₀ alkylene-, —C₁-C₁₀ heteroalkylene-, —C₃-C₈ carbocyclo-, —O—(C₁ C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀ heteroalkylene-C(═O)—, —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈carbocyclo-S—, —O—(C₁-C₈ alkyl)-)—S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, —(C₃-C₈ heterocyclo)-C₁-C₁₀ alkylene-S—, —C₁-C₁₀ alkylene-O—C(═O)—, —C₃-C₈ carbocyclo-O—C(═O)—, —O—(C₁-C₈ alkyl)-O—C(═O)—, -arylene-O—C(═O)—, —C₁-C₁₀ alkylene-arylene-O—C(═O)—, -arylene-C₁-C₁₀ alkylene-O—C(═O)—, —C₁-C₁₀ alkylene-(C₃-C₈carbocyclo)-O—C(═O)—, —(C₃-C₈ carbocyclo)-C₁-C₁₀ alkylene-O—C(═O)—, —C₃-C₈ heterocyclo-O—C(═O)—, —C₁-C₁₀ alkylene-(C₃-C₈heterocyclo)-O—C(═O)—, —(C₃-C₈ heterocyclo)-C₁-C₁₀ alkylene-O— C(═O)—.

Any of the R₆ group is optionally substituted with one or more of the substituents selected from: —X, —R′, —O⁻, —OR′, ═O, —SR′, —S—, —NR′₂, —NR′₃⁺, ═NR′, —CX₃, —CN, —OCN, —SCN, —N═C═O, —NCS, —NO, —NO₂, ═N₂, —N₃, —NR′C(═O)R′, —C(═O)R′, —C(═O)NR′₂, —SO₃—, —SO₃H, —S(═O)₂R′, —OS(═O)₂OR′, —S(═O)₂NR′, —S(═O)R′, —OP(═O)(OR′)₂, —P(═O)(OR′)₂, —PO₃—, —PO₃H₂, —C(═O)X, —C(═S)R′, —CO₂R′, —CO₂, —C(═S)OR′, C(═O)SR′, C(═S)SR′, C(═O)NR′₂, C(═S)NR′₂, and C(═NR′)NR′₂, where each X is independently a halogen: —F, —CI, —Br, or —I; and each R′ is independently —H, —C₁C₂₀ alkyl, —C₆-C₂₀ aryl, or —C₃-C₁₄ heterocycle.

Advantageously, the spacer unit is of formula (XVII), (XVIII), (XIX), (XX), (XXI) or (XXII),

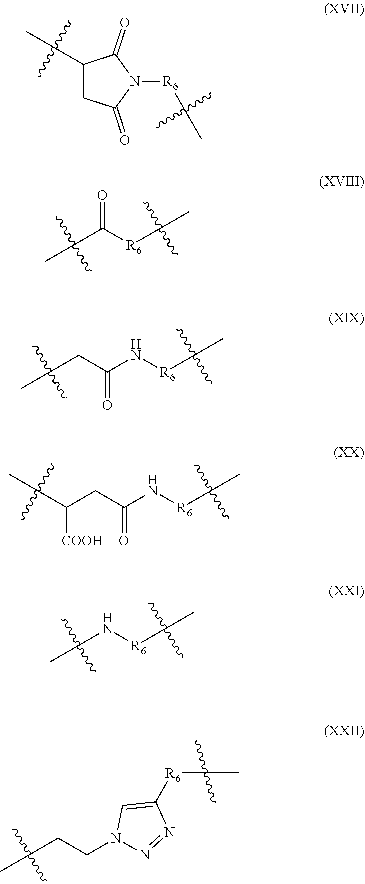

wherein the wavy bonds represent the attachment points and R₆ is —C₁-C₁₀ alkylene-, —C₁-C₁₀ heteroalkylene-, —C₁-C₁₀ alkylene-C(═O)—, —C₁-C₁₀ heteroalkylene-C(═O)—, -arylene-C₁-C₁₀ alkylene-C(═O)—, -arylene-C₁-C₁₀ alkylene-O—C(═O)—.

Any of the R₆ group is optionally substituted with one or more ═O.

A ligand refers to any macromolecule (polypeptide, protein, peptides, typically antibodies) as usually employed in LDC (e.g. Antibody Drug Conjugates) technologies, or to a small-molecule such as folic acid or an aptamer, that may be covalently conjugated with synthetic linkers or drug-linkers of the present work, using bioconjugation techniques (see Greg T. Hermanson, Bioconjugate Techniques, 3rd Edition, 2013, Academic Press). The ligand is traditionally a compound that is selected for its targeting capabilities. Non-exhaustive listing of ligand includes: proteins, polypeptides, peptides, antibodies, full-length antibodies and antigen-binding fragments thereof, interferons, lymphokines, hormones, growth factors, vitamins, transferrin or any other cell-binding molecule or substance. The main class of ligand used to prepare conjugates are antibodies. The term “antibody” as used herein is used in the broadest sense and covers monoclonal antibodies, polyclonal antibodies, modified monoclonal and polyclonal antibodies, monospecific antibodies, multispecific antibodies such as bispecific antibodies, antibody fragments and antibody mimetics (Affibody®, Affilin®, Affimer®, Nanofitin®, Cell Penetrating Alphabody®, Anticalin®, Avimer®, Fynomer®, Monobodies or nanoCLAMP®). An example of an antibody is trastuzumab. An example of protein is human serum albumin.

The term “antibody” as referred to herein includes whole antibodies and any antigen binding fragments (i.e., “antigen-binding portion”) or single chains thereof.

A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antigen portion”), as used herein, refers to full length or one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR), or any fusion proteins comprising such antigen-binding portion.

Furthermore, although the two domains of the Fv fragment, V_{L} and V_{H}, are coded for by separate genes, they can be joined, using recombinant methods, by a synthetic linker that enables them to be made as a single chain protein in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

In specific embodiments, the ligand of the LDC is a chimeric, humanized or human antibody.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutant versions of human germline sequences or antibody containing consensus framework sequences derived from human framework sequences analysis, for example, as described in Knappik, et al. (2000. J Mol Biol 296, 57-86).

The human antibodies may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences.

As used herein, “isotype” refers to the antibody class (e.g., IgM, IgE, IgG such as IgGI or IgG4) that is provided by the heavy chain constant region genes.

The phrases “an antibody recognizing an antigen” and “an antibody specific for an antigen” are used interchangeably herein with the term “an antibody which binds specifically to an antigen”.

A cleavable group (X), also referred as “releasable assembly unit”, links the drug unit to the remainder of the ligand-drug-conjugate. The cleavable group function is to release the drug at the site targeted by the ligand. This unit is thus capable of forming a cleavable linkage for the drug unit release, for example upon enzymatic treatment or disulfide elimination mechanism. The recognition site for enzymatic treatment is usually a dipeptide cleavage site (e.g. Val-Cit, Val-Ala or Phe-Lys) or a sugar cleavage site (e.g. glucuronide cleavage site). For example, a cleavable group is a glucuronide group. This technique is well-known to the one skilled in the art and from his knowledge, he is capable to select a cleavable group which is appropriate to the drug of the LDC (e.g. ADC) compound. For example, cleavable groups include disulfide containing linkers that are cleavable through disulfide exchange, acid-labile linkers that are cleavable at acidic pH, and linkers that are cleavable by hydrolases (e.g., peptidases, esterases, and glucuronidases). The cleavable group can be selected form
one or more natural or non-natural amino acids, for example a cleavable peptide comprising 2 to 12 amino acids,
a sugar moiety linked via an oxygen glycosidic bond to a self immolative group,
a disulfide linker, and
an acid-labile linker that is hydrolysable in the lysosome.

Advantageously, the cleavable group can be selected form
one or more natural or non-natural amino acids, for example a cleavable peptide comprising 2 to 12 amino acids, and
a sugar moiety linked via an oxygen glycosidic bond to a self immolative group,

When a sugar moiety is used, the self-immolative group is considered to be part of the cleavable group. The “self-immolative group” is a tri-functional chemical moiety that is capable of covalently linking together three spaced chemical moieties, i.e., the sugar moiety (via a glycosidic bond), the Drug D (directly or indirectly via a spacer Z), and the orthogonal connector L (directly or indirectly via a spacer Z). The glycosidic bond can be one that can be cleaved at the target site to initate a self-immolative reaction sequence that leads to a release of the drug.

When a disulfide linker is used, the cleavage occurs between the two sulfur atoms of the disulfide. A variety of disulfide linkers are known in the art and can be adapted for use in the present disclosure, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate), SMPT (N-succinimidyl-oxycarbonyl-alpha-methyl-alpha-(2-pyridyl-dithio)toluene), and SPP (N-succinimidyl 4-(2-pyridyldithio)pentanoate). See, for example U.S. Pat. No. 4,880,935.

In some embodiments, the Cleavable Unit is pH-sensitive and will comprise, for example, an acid-labile linker that is hydrolyzable in the lysosome (e.g., a hydrazone, semicarbazone, thiosemicarbazone, cis-aconitic amide, orthoester, acetal, or ketal group) can be used. (See, e.g., U.S. Pat. Nos. 5,122,368; 5,824,805; 5,622,929). Such linkers are relatively stable under neutral pH conditions, such as those in the blood, but are unstable at pH 5.5 or 5.0, the approximate pH of the lysosome.

A ligand drug conjugate (LDC) refers to any conjugate that binds a ligand and a drug as defined above and involving any mean such as described above, and that will be illustrated in the examples of the description. When the ligand is an antibody, one may refer to antibody drug conjugate (ADC) which is a preferred embodiment of the present disclosure.

A bindable group refers to a group that can react with the functionalized reactive group to form a covalent bond. The bindable group thus comprises a reactive group which reacts with the functionalized reactive group in determined reaction conditions. In particular, the bindable group can comprise one of the following group: carboxylic acid; primary amine; secondary amine; tertiary amine; hydroxyl; halogen; activated ester such as N-hydroxysuccinimide ester, perfluorinated esters, nitrophenyl esters, aza-benzotriazole and benzotriazole activated ester, acylureas; alkynyl; alkenyl; azide; isocyanate; isothiocyanate; aldehyde; thiol-reactive moieties such as maleimide, halomaleimides, haloacetyls, pyridyl disulfides; thiol; acrylate; mesylate; tosylate; triflate, hydroxylamine; chlorosulfonyl; boronic acid —B(OR′)₂ derivatives wherein R′ is hydrogen or alkyl group.

A drug refers to any type of drug or compounds, for example cytotoxic, cytostatic, immunosuppressive, anti-inflammatory or anti-infective compounds. Among cytotoxic compounds, one can cite calicheamicins; uncialamycins; auristatins (such as monomethyl auristatin E known as MMAE); tubulysin analogs; maytansines; cryptophycins; benzodiazepine dimers (including Pyrrolo[2,1-c][1,4]benzodiazepines known as PBD's); indolinobenzodiazepines pseudodimers (IGNs); duocarmycins; anthracyclins (such as doxorubicin or PNU159682); camptothecin analogs (such as 7-Ethyl-10-hydroxy-camptothecin known as SN38 or exatecan); Bc12 and Bc1-x1 inhibitors; thailanstatins; amatoxins (including α-amanitin); kinesin spindle protein (KSP) inhibitors; vinorelbine; cyclin-dependent kinase (CDK) inhibitors; bleomycin; dactinomycin or radionuclides and their complexing agent (such as DOTA/¹⁷⁷Lu). Among anti-inflammatory drugs, one can cite corticosteroids such as dexamethasone or fluticasone. Among anti-infective drugs, one can cite antibiotics such as rifampicin or vancomycin.

The present invention is now exposed in more details. Although it is more specifically described in reference to a single-molecular-weight of polysarcosine homopolymer, it should be acknowledged its scope extends to any single-molecular-weight that are encompassed within formula (I) above. Moreover, the benefits of the invention are specifically evidenced in LDC technology. Of course, its advantages are not restricted to such technology and in any area where a single-molecule-weight, biocompatible, biodegradable homopolymer is needed, it can exhibit similar or better performances.

Thus, the present invention more particularly pertains to a single molecular weight homopolymer of sarcosine, having formula (II)

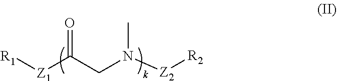

wherein
R₁ and R₂ are different, and
one of R₁ and R₂ is H or an inert group, the other one of R₁ and R₂ being a functionalized reactive group, said group being reactive for covalently binding a bindable group, in such reaction conditions that the inert group is non-reactive,
Z₁ and Z₂, identical or different, are optional spacers, and
k is 2 or more.

Further features of a homopolymer of formula (I), in particular of a homopolymer of formula (II) are given below, taken alone or in any combination.
k is an integer which is at least 2, it is preferably 100 at most, more preferably 50 at most, and specifically 2-30, and more specifically 2-24, 6-24, or 12-24.

In either formula (I) or formula (II), said functionalized reactive group R₁ or R₂ may be selected from the following groups:
carboxylic acid group,
amino groups NRR″ wherein R and R″ are independently selected from H, (C₁-C₆) alkyl optionally interrupted by at least one heteroatom selected among O, N and S,
hydroxyl group,
halogen atoms,
hydrazine (—NH₂—NH₂) group,
nitro group,
hydroxylamine group,
azido group,
(C₂-C₆) alkynyl group,
(C₂-C₆) alkenyl group,
thiol group,
activated ester groups such as N-hydroxysuccinimide ester, perfluorinated esters, nitrophenyl esters, aza-benzotriazole and benzotriazole activated esters, acylureas,
boronic acid —B(OR″″)₂ groups, wherein R″″ is a hydrogen atom or a C₁-C₆ alkyl group,
thiol-reactive groups such as maleimide, halomaleimides, haloacetyls, pyridyl disulfides,
mesylate group,
tosylate group,
triflate group,
aldehyde group,
isocyanate or isothiocyanate group,
chlorosulfonyl group,
acrylate group.

As mentioned above, spacers Z are optional, both Z₁ and Z₂ may be present, only one of Z₁ and Z₂ may be present, they also may not be present. In this latter case and when the homopolymer of the invention is a homopolymer of sarcosine, it has formula (III)

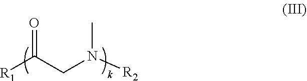

wherein R₁, R₂ and k are as defined above.

In formula (I), formula (II) or formula (III), R₁ may be H or an inert group and R₂ a functionalized reactive group or R₁ may be a functionalized reactive group and R₂ H or an inert group.

According to a preferred embodiment, the functionalized reactive group R₁ or R₂ is a secondary amine and the inert group R₁ or R₂ is a carboxylic acid that remains unreacted and unbound on the final LDC structure.

In a preferred embodiment, in formula (I), formula (II) or formula (III), R₁ is selected from OH and NH₂, and
when R₁ is OH, R₂ is COCH₃ and
when R₁ is NH₂, R₂ is CO-G-COOH, G being CH₂CH₂, CH₂CH₂CH₂, CH₂CH₂CH₂CH₂, CH₂OCH₂, CH₂SCH₂, CH₂CH(CH₃)CH₂, CH₂C(CH₃)₂CH₂ or CH₂N(CH₃)CH₂.

The present invention also pertains to methods for preparing a single-molecular-weight-homopolymer of either formula (I), formula (II) or formula (III). Generally, each N-methylglycine monomer is assembled on a solid-support from two sub-monomers, namely a haloacetic acid and methylamine. Each monomer addition is referred to as a cycle of: (i) acylation of the resin-bound secondary amine with haloacetic acid and a carbodiimide or other suitable carboxylate activation method, (ii) washing steps, (iii) nucleophilic displacement of the resin-bound halogen with methylamine, (iv) washing steps.

A method in accordance with the invention comprising the following steps:
a) Reacting a compound of formula (IV)

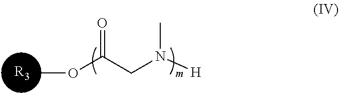

wherein R₃ is a peptide synthesis solid phase support, and m is 1 or more and being less than k,
with an acid of formula (V)

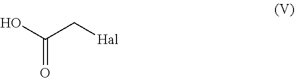

wherein Hal is halogen,
to obtain a compound of formula (VI)

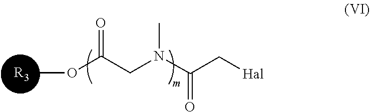

wherein R₃, m and Hal are as defined above,
b) Reacting said compound of formula (VI) with methylamine
to obtain a compound of formula (VII)

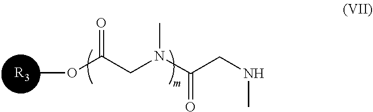

wherein R₃ and m are as defined above,
c) Reiterating steps a) and b) until a compound of formula (VIII) is obtained,

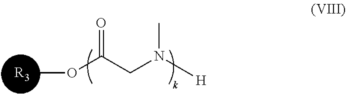

wherein R₃ is defined above and k is as defined above,
d) Reacting said compound (VIII) to obtain a compound of formula (IX)

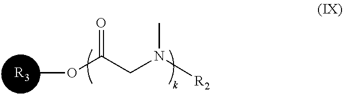

wherein R₂ is an inert group, R₃ is defined above and k is as defined above,
e) Cleavage reaction to obtain a single molecule weight homopolymer of formula (III) as defined above.

In accordance with an embodiment of this method, it comprises in step a), reacting a compound of formula (IV) wherein R₃ is a peptide synthesis solid phase support and m is 3, said compound being obtained by Fmoc- solid-phase peptide synthesis methodologies. They are well-known to the one skilled in the art, and from his knowledge, he is capable to select any suitable coupling reagent, for example N—[(Dimethylamino)-1H-1,2,3-triazolo-[4,5-b]pyridin-1-ylmethylene]-N-methylmethanaminium hexafluorophosphate N-oxide (HATU).

In an alternative embodiment of a method of the invention, preparing a single molecular weight homopolymer, comprises the following steps:
a) Reacting a compound of formula (X)

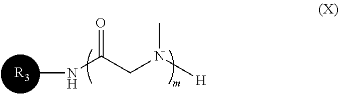

wherein R₃ is a peptide synthesis solid phase and m is 1 or more and being less than k,
with an acid of formula (V)

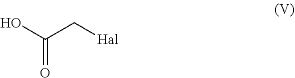

wherein Hal is halogen,
to obtain a compound of formula (XI)

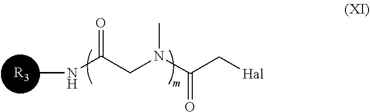

wherein R₃, m and Hal are as defined above,
b) Reacting said compound of formula (XI) with methylamine
to obtain a compound of formula (XII)

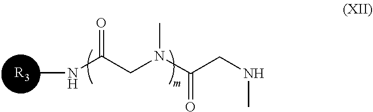

wherein R₃ and m are as defined above,
c) Reiterating steps a) and b) until a compound of formula (XIII) is obtained,

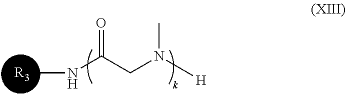

wherein R₃ and k are as defined above,
d) Cleavage reaction to obtain a compound of formula (XIV)

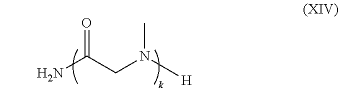

wherein k is as defined above,
e) Reacting said compound (XIV) with at least one of succinic anhydride, glutaric anhydride, adipic anhydride, diglycolic anhydride, thiodiglycolic anhydride, 3-methylglutaric anhydride, 3-3-dimethylglutaric anhydride or 4-methylmorpholine-2,6-dione, to obtain a single molecule weight homopolymer of formula (III) as defined above.

As previously mentioned, a homopolymer of the invention is useful for LDC technology, without being restricted to this technology.

Thus, the invention also related to a Ligand-Drug-Conjugate compound (LDC) having the following formula (XV)

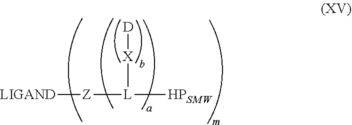

wherein
L is an orthogonal connector that allows for (HP_{SMW}) to be in an orthogonal orientation with respect to (X-D),
HP_{SMW} results from covalent binding of a single molecular weight homopolymer of the invention as described above, to said orthogonal connector L,
D is a drug, particularly a cytotoxic drug, such as monomethyl auristatin E (MMAE), or SN38,
X is an optional cleavable moiety for releasing D,
Z is an optional spacer, and
a is 1 or more, b is 1 or more and m is 1 or more.

The single molecular weight homopolymer, and in particular the single molecular weight polysarcosine, when grafted in parallel (i.e. orthogonal) orientation in relation to the drug unit provides efficient hydrophobicity masking properties, reduced apparent hydrophobicity, better pharmacokinetics properties, and improved in vivo activity of the conjugate compared to ligand-drug-conjugate comprising no single molecular weight homopolymer grafted in parallel.

In an alternative embodiment, D is selected from the group consisting of a bioactive molecule, a therapeutic molecule such as an anticancer drug, an imaging agent and a fluorophore.

In accordance with alternative embodiments of this invention:
a is an integer which is at least 1, preferably 6 at most, more preferably 3 at most, and specifically 2, and more specifically 1, and/or
b is an integer which is at least 1, preferably 6 at most, more preferably 3 at most, and specifically 2, and more specifically 1, and/or
m is an integer which is at least 1, preferably 30 at most, more preferably 15 at most, and specifically 8, and more specifically 4.

Advantageously, the single molecular weight homopolymer is polysarcosine.

In one embodiment, there is a spacer Z between L and LIGAND, and/or between L and HP_{SMW} and/or between L and X, and/or between X and D.

Typically, the orthogonal connector connects a releasable assembly-drug unit (X-D) or a drug unit (D) through one or more linker unit components, in such a manner that the (X-D) or (D) unit are in a parallel configuration (as opposed to in series configuration) in relation to the homopolymer unit.

The invention also pertains to an intermediate compound having formula (XVI)

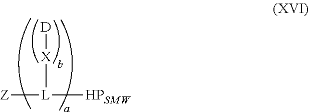

wherein
L is an orthogonal connector,
HP_{SMW} results from covalent binding of a single molecular weight homopolymer of the invention, to said orthogonal connector L,
D is a cytotoxic drug,
X is an optional cleavable moiety for releasing D,
Z is an optional spacer, said spacer being able to bind a ligand, and
a is 1 or more and b is 0, 1 or more.

The present disclosure also relates to a compound having the formula (XXIII)

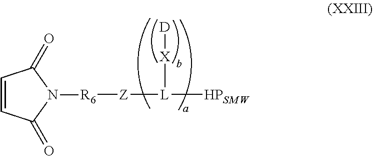

wherein
R₆ is —C₁-C₁₀ alkylene-, —C₁-C₁₀ heteroalkylene-, —C₃-C₈ carbocyclo-, —O—(C₁ C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀ heteroalkylene-C(═O)—, —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈carbocyclo-S—, —O—(C₁-C₈ alkyl)-)—S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, —(C₃-C₈ heterocyclo)-C₁-C₁₀ alkylene-S—, —C₁-C₁₀ alkylene-O— C(═O)—, —C₃-C₈ carbocyclo-O—C(═O)—, —O—(C₁-C₈ alkyl)-O—C(═O)—, -arylene-O—C(═O)—, —C₁-C₁₀ alkylene-arylene-O—C(═O)—, -arylene-C₁-C₁₀ alkylene-O—C(═O)—, —C₁-C₁₀ alkylene-(C₃-C₈carbocyclo)-O—C(═O)—, —(C₃-C₈ carbocyclo)-C₁-C₁₀ alkylene-O— C(═O)—, —C₃-C₈ heterocyclo-O—C(═O)—, —C₁-C₁₀ alkylene-(C₃-C₈heterocyclo)-O—C(═O)—, —(C₃-C₈ heterocyclo)-C₁-C₁₀ alkylene-O—C(═O)—,
any of the R₆ group is optionally substituted with one or more of the substituents selected from: —X, —R′, —O⁻, —OR′, ═O, —SR′, —S—, —NR′₂, —NR′, ═NR′, —CX₃, —CN, —OCN, —SCN, —N═C═O, —NCS, —NO, —NO₂, ═N₂, —N₃, —NR′C(═O)R′, —C(═O)R′, —C(═O)NR′₂, —SO₃—, —SO₃H, —S(═O)₂R′, —OS(═O)₂OR′, —S(═O)₂NR′, —S(═O)R′, —OP(═O)(OR′)₂, —P(═O)(OR′)₂, —PO₃⁻, —PO₃H₂, —C(═O)X, —C(═S)R′, —CO₂R′, —CO₂, —C(═S)OR′, C(═O)SR′, C(═S)SR′, C(═O)NR′₂, C(═S)NR′₂, and C(═NR′)NR′₂, where each X is independently a halogen: —F, —CI, —Br, or —I; and each R′ is independently —H, —C₁C₂₀ alkyl, —C₆-C₂₀ aryl, or —C₃-C₁₄ heterocycle,
Z is an optional spacer,
L is an orthogonal connector,
X is an optional cleavable moiety for releasing D,
D is a cytotoxic drug,
a is 1 or more and b is 0, 1 or more, and
HP_{SMW} results from covalent binding of a single molecular weight homopolymer of the invention, to said orthogonal connector L are as defined above.

In accordance with a preferred embodiment, HP_{SMW} results from covalent binding of a polysarcosine homopolymer of the invention, to said orthogonal connector L. In this case, in formulae (XV), (XVI) and (XXIII), HP_{SMW} represents

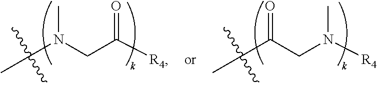

wherein the wavy bond represents the attachment point to L or to a spacer Z, if present,
k is 2 or more, preferably k is 2 to 50, and
R₄ represent a capping group.

Advantageously, R₄ represents —R′, —O⁻, —OR′, —SR′, —S⁻, —NR′₂, —NR′₃+, ═NR′, —CX₃, —CN, —NRC(═O)R′, —C(═O)R′, —C(═O)NR′2, —SO₃—, —SO₃H, —S(═O)₂R′, —OS(═O)₂OR′, —S(═O)₂NR′, —S(═O)R′, —OP(═O)(OR′)₂, —P(═O)(OR′)₂, —PO₃⁻, —PO₃H₂, —C(═O)X, —C(═S)R′, —CO₂R′, —CO₂, —C(═S)OR′, C(═O)SR′, C(═S)SR′, C(═O)NR′₂, C(═S)NR′₂, or C(═NR′)NR′₂, where each X is independently a halogen: —F, —CI, —Br, or —I, and each R′ is independently —H, —C₁C₂₀ alkyl, —C₆-C₂₀ aryl, or —C₃-C₁₄ heterocycle. Typically, R₄ is —OR′, —NR′₂, or —C(═O)R′.

In one embodiment, the disclosure also relates to a Ligand-Drug-Conjugate compound (LDC) having the following formula (XV)

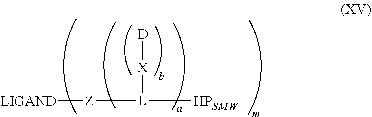

wherein
the LIGAND is an antibody,
L is an orthogonal connector that allows for HP_{SMW} to be in an orthogonal orientation with respect to (X-D), selected from natural or non-natural aminoacids, amino alcohols; amino aldehydes; polyamines and combination thereof,
HP_{SMW} represents

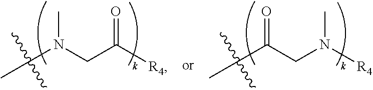

wherein the wavy bond represents the attachment point to L or to a spacer Z, if present,
k is 2 or more, preferably k is 2 to 50, and
R₄ represent a capping group,
D is a drug, particularly a cytotoxic drug, such as monomethyl auristatin E (MMAE), or SN38,
X is an optional cleavable moiety for releasing D, selected form
   one or more natural or non-natural amino acids, for example a cleavable peptide comprising 2 to 12 amino acids,
   a sugar moiety linked via an oxygen glycosidic bond to a self immolative group,
   a disulfide linker, and
   an acid-labile linker that is hydrolysable in the lysosome
Z is an optional spacer, which can also be present between L and X, and/or X and D, and/or L and HP_{SMW}, and is selected from alkylene, heteroalkylene; alkoxy; polyether; one or more natural or non-natural aminoacids; C₃-C₈ heterocyclo; C₃-C₈ carbocyclo; arylene, and any combination thereof,
a is 1 or more, b is 1 or more and m is 1 or more.

The invention also relates to a pharmaceutical composition comprising at least one LDC compound of the invention and a pharmaceutically acceptable carrier.

The present disclosure also relates to a LDC compound as described above, for use as a medicament.

The compound of formula (XXIII) can be used as such without the ligand as the maleimide moiety can react in vivo with a protein, like serum albumin, which then becomes the ligand. Thus, the present disclosure also relates to a compound of formula (XXIII) as described above, for use as a medicament.

### BRIEF DESCRIPTION OF THE FIGURES

FIG. 1 represents the hydrophobic interaction chromatogram according to example 12.

FIG. 2 represents the hydrophobic interaction chromatogram according to example 13.

FIG. 3 represents the pharmacokinetic profile in mice according to example 14.

FIG. 4A represents the tumor volume in function of time according to example 15.

FIG. 4B represents the survival percentage of mice according to example 15.

FIG. 5 represents the pharmacokinetic profile in mice according to example 16.

FIG. 6 represents the tumor volume in function of time according to example 17.

### EXAMPLES

### Materials and General Methods

All solvents and reagents were obtained from commercial sources (Sigma-Aldrich, Alfa Aesar, Fluorochem, Thermo Fisher, Carbosynth) and used without further purification unless stated otherwise. Anhydrous DMF and DCM were purchased from Sigma-Aldrich. Fmoc-aminoacids, 2-chlorotrityl and Rink amide resins were purchased from Novabiochem. Monomethyl auristatin E (MMAE) and 7-ethyl-10-hydroxycamptothecin (SN38) were purchased from DCChemicals. PNU159682 was purchased from Kerui Biotechnology Co. Ltd. and Exatecan mesylate was purchased from Angene Chemical. Human albumin (cat #A3782) was purchased from Sigma-Aldrich. Anti-CD19 and anti-CD22 antibodies were purchased from Euromedex.

Trastuzumab (Herceptin® IV) was purchased from Roche. On-resin synthesis was performed in empty SPE plastic tubes equipped with a 20 μm polyethylene frit (Sigma-Aldrich). A Titramax 101 platform shaker (Heidolph) was used for agitation. Unless stated otherwise, all chemical reactions were carried out at room temperature under an inert argon atmosphere.

Liquid nuclear magnetic resonance spectra were recorded on a Bruker Fourier 300HD spectrometer, using residual solvent peak for calibration. Mass spectroscopy analysis has been performed by the Centre Commun de Spectrometrie de Masse (CCSM) of the UMR5246 CNRS institute of the University Claude Bernard Lyon 1.

Normal phase flash chromatography was performed on a Teledyne Isco CombiFlash® Companion® device or Teledyne Isco CombiFlash® Rf200 device using either Interchim (spherical HP 50 μm) or Biotage® ZIP® (50 μm) silica cartridges. Reverse phase chromatography was performed using Biotage® SNAP Ultra C₁₈ (25 μm) cartridges or Interchim PuriFlash RP-AQ (30 μm) cartridges. Chemical reactions and compound characterization were respectively monitored and analyzed by thin-layer chromatography using pre-coated 40-63 μm silica gel (Macherey-Nagel), HPLC-UV (Agilent 1050) or UHPLC-UV/MS (Thermo UltiMate 3000 UHPLC system equipped with a Bruker Impact II™ Q-ToF mass spectrometer or Agilent 1260 HPLC system equipped with a Bruker MicrOTOF-QII mass spectrometer).

HPLC Method 1: Agilent 1050 equipped with DAD detection. Mobile phase A was water and mobile phase B was acetonitrile. Column was an Agilent Zorbax SB-Aq 4.6×150 mm 5 μm (room temperature). Gradient was 5% B to 95% B in 20 min, followed by a 5 min hold at 95% B. Flow rate was 1.5 mL/min. UV detection was monitored at 214 nm.

HPLC Method 2: Agilent 1050 equipped with DAD detection. Mobile phase A was water and mobile phase B was acetonitrile. Column was an Agilent Zorbax SB-Aq 4.6×150 mm 5 μm (room temperature). Gradient was 0% B to 50% B in 30 min, followed by a 5 min hold at 50% B. Flow rate was 1.0 mL/min. UV detection was monitored at 214 nm.

HPLC Method 3: Same as HPLC Method 1 but contains 0.1% TFA into the mobile phase A.

HPLC Method 4: Same as HPLC Method 2 but contains 0.1% TFA into the mobile phase A.

UHPLC Method 5: Thermo UltiMate 3000 UHPLC system+Bruker Impact II™ Q-ToF mass spectrometer. Mobile phase A was water+0.1% formic acid and mobile phase B was acetonitrile+0.1% formic acid. Column was an Agilent PLRP-S 1000Å 2.1×150 mm 8 μm (80° C.). Gradient was 10% B to 50% B in 25 min. Flow rate was 0.4 mL/min. UV detection was monitored at 280 nm. The Q-ToF mass spectrometer was used in the m/z range 500-3500 (ESIJ. Data were deconvoluted using the MaxEnt algorithm included in the Bruker Compass® software.

HPLC Method 6: Agilent 1050 equipped with DAD detection. Mobile phase A was water+5 mM ammonium formate and mobile phase B was acetonitrile. Column was an Agilent Poroshell 120 EC-C18 3.0×50 mm 2.7 μm (room temperature). Gradient was 5% B to 90% B in 10 min, followed by a 2 min hold at 90% B. Flow rate was 0.8 mL/min. UV detection was monitored at 214 nm.

### Examples 1-4 Below Illustrate Synthesis of a Single Molecular Weight Polysarcosine of the Invention, this Synthesis being Part of the Invention, Involving Different Solid-Phase Synthesis Methodologies

### Example 1: Synthesis of Polysarcosine Compounds (On-Resin Synthesis Method 1)

The reaction scheme is mentioned below.

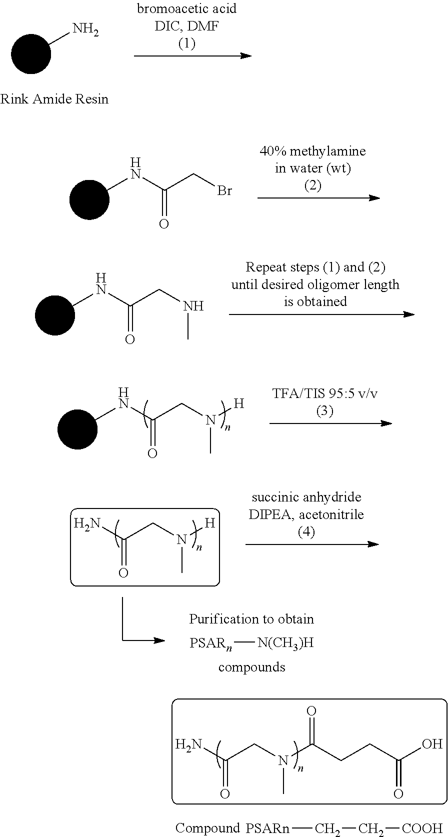

### 1.1) General Methods

On-resin synthesis was performed in empty SPE plastic tubes equipped with a 20 μm polyethylene frit (Sigma-Aldrich). A Titramax 101 platform shaker (Heidolph) was used for agitation. All synthesis yields reported are based upon an initial theoretical resin loading of 0.63 mmol/g (extent of labeling indicated by the manufacturer). Unless stated otherwise, all reactions were carried out at room temperature.

### 1.2) Resin Loading

Typically, 500 mg of NovaGEL™ Rink Amide beads (0.63 mmol/g, Novabiochem) were swollen in 5 mL of DMF for 15 min. The first monomer was added by reacting 10 eq of bromoacetic acid and 13 eq of diisopropylcarbodiimide (Sigma-Aldrich) in 5 mL of DMF for 60 min at room temperature, followed by extensive washes with DMF (5 times 5 mL). Bromoacetylated resin was incubated for 30 min with 5 mL of a 40% (wt) methylamine in water solution (Sigma-Aldrich) on a shaker platform, followed by extensive washes with DMF (5 times 5 mL) and DCM (5 times 5 mL). The obtained resin was ready for elongation.

### 1.3) Elongation of Polysarcosine Compounds

Elongation of the polysarcosine oligomer was performed until the desired length was obtained, by alternating bromoacetylation and amine displacement steps. The bromoacetylation step was performed by adding 10 eq of bromoacetic acid and 13 eq of diisopropylcarbodiimide in 5 mL of DMF. The mixture was agitated for 30 min, drained and washed with DMF (4 times 5 mL). For the amine displacement step, 5 mL of a 40% (wt) methylamine in water solution (Sigma-Aldrich) was added and the vessel was shaken for 30 min, drained and washed with DMF (4 times 5 mL) and DCM (4 times 5 mL).

### 1.4) Cleavage from the Resin

Cleavage of the polysarcosine oligomer was performed using 5 mL of a TFA/triisopropylsilane (95:5) solution at room temperature under agitation. The resin was filtered and the obtained solution was evaporated under reduced pressure to give an oily transparent material.

At this stage, PSARn-N(CH₃)H were dissolved in water for purification (see below) or engaged into final functionalization.

### 1.5) Final Functionalization

To obtain PSARn-CH₂—CH₂—COOH compounds, the N-terminal end of the oligomer was functionalized using 2.5 eq of succinic anhydride and 10 eq of DIPEA in anhydrous acetonitrile. The mixture was stirred 1 hour at room temperature and volatiles were removed under reduced pressure.

### 1.6 Purification

PSAR compounds were purified on Interchim® RP-AQ (30 μm) cartridges. Mobile phase A was water+0.05% TFA and mobile phase B was acetonitrile+0.05% TFA. The gradient ranged from 0 to 30% B.

### 1.7) Single Molecular Weight Polysarcosine Compounds

The following table 1 lists the resulting PSAR compounds.

TABLE 1

| | | | | HPLC Method |
|---|---|---|---|---|
| Compound | | | | 2 retention |
| Name | Structure | Yield | HRMS (ESI⁺) | time |
| PSAR6- CH₂CH₂—COOH (transparent oil) | 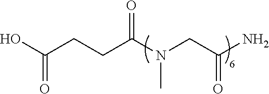 | 18% | Calc [M + H]⁺ = 544.2726 Exp [M + H]⁺ = 544.2730 Error = −0.9 ppm. | 7.3 min |
| PSAR12- CH₂—CH₂—COOH (white thick wax aspect) | 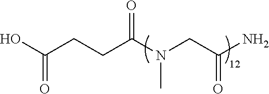 | 12% | Calc [M + H]⁺ = 970.4952 Exp [M + H]⁺ = 970.4951 Error = 0.2 ppm. | 9.2 min |
| PSAR18- CH₂—CH₂—COOH (white thick wax aspect) | 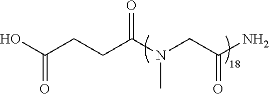 | 8% | Calc [M + Na]⁺ = 1418.699 Exp [M + Na]⁺ = 1418.7032 Error = −2.3 ppm. | 10.6 min |
| PSAR8- N(CH₃)H (transparent oil) | 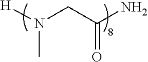 | 17% | Calc [M + H]⁺ = 586.3307 Exp [M + H]⁺ = 586.3284 Error = | 9.9 min |
| | | | 4.0 ppm. | |
| PSAR12- N(CH₃)H (white thick wax aspect) | 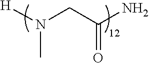 | 14% | Calc [M + H]⁺ = 870.4792 Exp [M + H]⁺ = 870.4784 Error = | 11.5 min |
| | | | 0.9 ppm. | |
| PSAR18- N(CH₃)H (white thick wax aspect) | 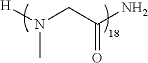 | 10% | Calc [M + 2H]²⁺ = 648.8546 Exp [M + 2H]²⁺ = 648.8563 Error = | 13.0 min |
| | | | −2.7 ppm. | |

### Example 2: Synthesis of Polysarcosine Compounds (On-Resin Synthesis Method 2)

The reaction scheme is mentioned below.

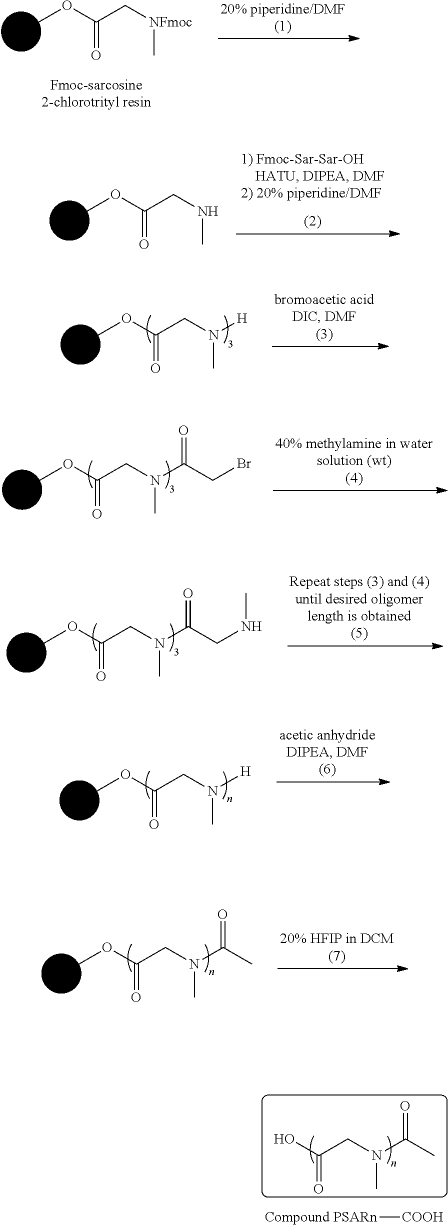

### 2.1) General Methods

On-resin synthesis was performed in empty SPE plastic tubes equipped with a 20 μm polyethylene frit (Sigma-Aldrich). A Titramax 101 platform shaker (Heidolph) was used for agitation. All synthesis yields reported are based upon an initial resin loading of 1.1 mmol/g (extent of labeling indicated by the manufacturer). Unless stated otherwise, all reactions were carried out at room temperature.

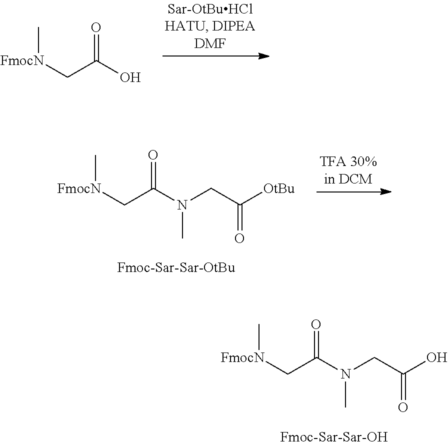

### 2.2.1) Synthesis of Fmoc-Sar-Sar-OtBu

Fmoc-Sar-OH (2000 mg/6.42 mmol) and HATU (2443 mg/6.42 mmol) were dissolved in 28 mL of anhydrous DMF in a round-bottom flask. DIPEA (2491 mg/19.27 mmol) was added and the mixture was stirred for 3 min at room temperature. Sarcosine tert-butyl ester hydrochloride (1167 mg/6.42 mmol) was then added and the reaction mixture was stirred at room temperature for 90 min. Volatiles were removed under vacuum and the residue was diluted with water and extracted 3 times with EtOAc. The organic phase was dried over MgSO₄, filtered and evaporated under vacuum to afford a solid crude. The crude was taken up in EtOAc/DCM 80:20 (v/v) and white insolubles were removed via filtration. The filtrate was purified by chromatography on silica gel (petroleum ether/EtOAc, gradient from 60:40 to 20:80) to afford Fmoc-Sar-Sar-OtBu (2310 mg/82%) as a white solid. HRMS m/z (ESI⁺): Calc [M+H]⁺=439.2227; Exp [M+H]+=439.2234; Error=−1.5 ppm. HPLC Method 1 retention time=13.3 min. TLC eluting with 100% EtOAc: Rf=0.8.

### 2.2.2) Tert-Butyl Ester Removal

Fmoc-Sar-Sar-OtBu (2310 mg/5.27 mmol) was dissolved in 20 mL of DCM and 8.5 mL of TFA was slowly added. The solution was stirred at room temperature until entire tert-butyl ester deprotection was observed by HPLC (approximately 2 hours). Volatiles were then removed under vacuum and the residue was triturated with diethyl ether to afford Fmoc-Sar-Sar-OH (1690 mg/84%) as a white solid. ¹H NMR (500 MHz, DMSO-d₆, 100° C.) δ (ppm) 2.84 (s, 3H), 2.93 (s, 3H), 4.01 (s, 2H), 4.05 (s, 2H), 4.25 (t, J=4.3 Hz, 1H), 4.34 (d, J=6.4 Hz, 2H), 7.33 (t, J=7.4 Hz, 2H), 7.41 (t, J=7.4 Hz, 2H), 7.63 (d, J=7.4 Hz, 2H), 7.85 (d, J=7.5 Hz, 2H). HRMS m/z (ESI⁺): Calc [M+H]⁺=383.1601; Exp [M+H]⁺=383.1602; Error=0.0 ppm. HPLC Method 1 retention time=6.2 min. TLC eluting with DCM/MeOH 85:15 (v/v): Rf=0.65.

### 2.3) Resin Loading

Typically, 1000 mg of 2-chlorotrityl chloride resin beads (100-200 mesh, 1% DVB, 1.1 mmol/g, Novabiochem) were swollen in 10 mL of DCM for 10 min. Fmoc-Sar-OH (1.2 eq), previously dissolved in 10 mL of dry DCM, was added onto the resin. DIPEA (5 eq) was added and the reaction vessel was agitated for 2 hours at room temperature. After draining, the resin was washed with DCM (3 times), DMF (2 times), DCM (3 times) and MeOH (2 times). The resin was dried under high vacuum overnight. Substitution level was assessed from the weight gain of the resin and/or from Fmoc cleavage test (absorbance measurement at 301 nm) and was found to be quasi-quantitative (usually 0.95-1.1 mmol/g). Resin was stored at −20° C. until further use.

### 2.4) Fmoc-Sar-Sar-OH Coupling Procedure

Resin was treated with 20% piperidine in DMF (1 mL per 100 mg of resin) for 2 times 15 min at room temperature. The resin was then washed with DMF (4 times) and DCM (4 times). To the resin was added a solution of Fmoc-Sar-Sar-OH (3 eq), HATU (2.85 eq) and DIPEA (6 eq) in DMF (1 mL per 100 mg of resin). The reaction vessel was agitated for 2 hours and the resin was extensively washed with DMF (5 times) and DCM (5 times). The resin was dried under vacuum and stored at −20° C. until further use.

### 2.5) Elongation of Polysarcosine Compounds

The resin was treated with 20% piperidine in DMF (1 mL per 100 mg of resin) for 2 times 15 min at room temperature. The resin was then washed with DMF (4 times) and DCM (4 times).

Elongation of the polysarcosine oligomer was performed until the desired length was obtained, by alternating bromoacetylation and amine displacement steps. The bromoacetylation step was performed by adding 10 eq of bromoacetic acid and 13 eq of diisopropylcarbodiimide in DMF (2 mL per 100 mg of resin). The mixture was agitated for 30 min, drained and washed with DMF (4 times). For the amine displacement step, a 40% (wt) methylamine in water solution was added (1.5 mL per 100 mg of resin) and the vessel was shaken for 30 min, drained and washed with DMF (4 times) and DCM (4 times).

### 2.6) Final Acetylation

When the desired oligomer length was obtained, the N-terminal end was acetylated using a capping solution made of acetic anhydride/DIPEA/DMF (1:2:3 v/v) (vessel shaken for 30 min). The solution was drained, and the reaction was repeated once with fresh capping solution. The resin was washed with DMF (4 times) and DCM (4 times).

### 2.7) Resin Cleavage

Cleavage of the polysarcosine oligomer from the resin was performed using a HFIP/DCM (20:80 v/v) solution under agitation for 30 minutes. Resin was filtered, and volatiles were removed under reduced pressure to afford a solid crude.

### 2.8) Purification

PSAR compounds were purified on Interchim® RP-AQ (30 μm) cartridges. Mobile phase A was water+0.1% TFA and mobile phase B was acetonitrile+0.1% TFA.

### 2.9) Single Molecular Weight Polysarcosine Compounds

The following table 2 lists the resulting PSAR compounds.

TABLE 2

| Compound | | | | HPLC Method 2 |
|---|---|---|---|---|
| Name | Structure | Yield | HRMS (ESI⁺) | retention time |
| PSAR6-COOH (transparent oil) | 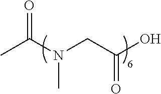 | 94% | Calc [M + H]⁺ = 487.2511 Exp [M + H]⁺ = 487.2504 Error = 1.5 ppm. | 8.0 min |
| PSAR12- COOH (white thick wax aspect) | 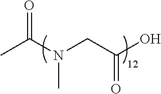 | 84% | Calc [M + H]⁺ = 913.4738 Exp [M + H]⁺ = 913.4720 Error = 2.0 ppm. | 8.7 min |
| PSAR18- COOH (white thick wax aspect) | 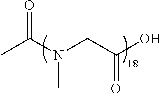 | 66% | Calc [M + Na]⁺ = 1361.6784 Exp [M + Na]⁺ = 1361.6759 Error = 1.8 ppm. | 9.8 min |
| PSAR24- COOH (white thick wax aspect) | 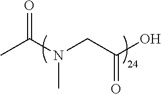 | 55% | Calc [M + 2Na]²⁺ = 905.4452 Exp [M + 2Na]²⁺ = 905.4470 Error = −2.0 ppm | 10.5 min |

### Example 3: Synthesis of Polysarcosine Compounds Bearing One or Several Azido-Functionalized Orthogonal Connector(s) (On-Resin Synthesis Method 3)

The reaction scheme is mentioned below.

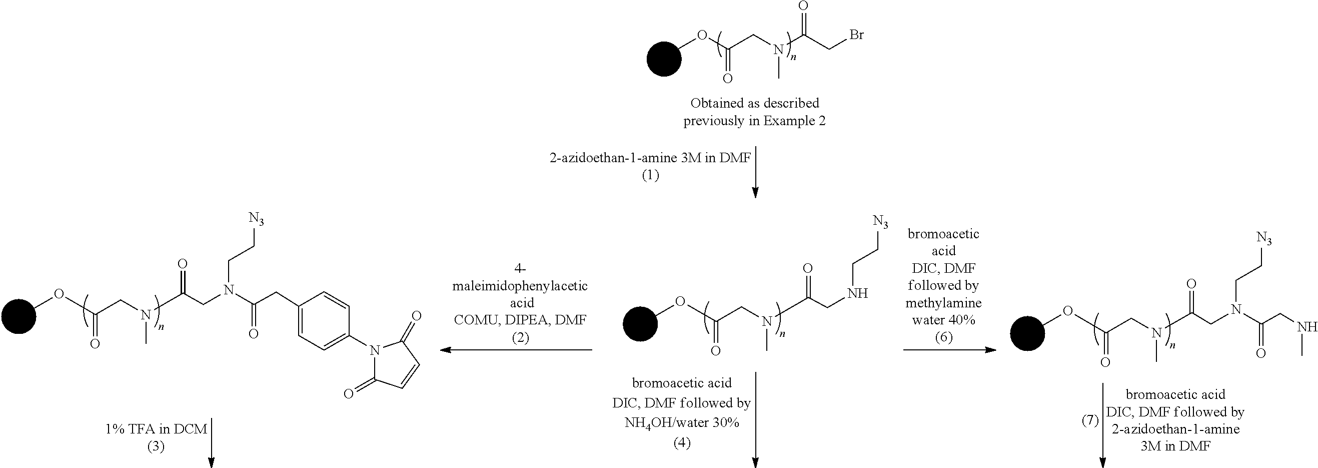

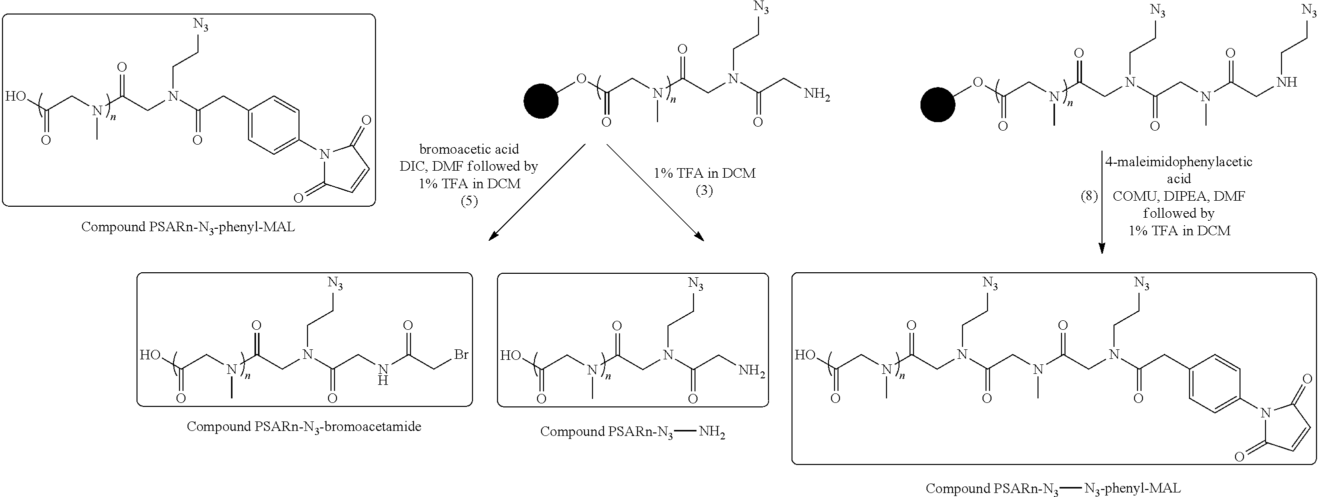

### 3.1) General Methods

On-resin synthesis was performed in empty SPE plastic tubes equipped with a 20 μm polyethylene frit (Sigma-Aldrich). A Titramax 101 platform shaker (Heidolph) was used for agitation. All synthesis yields reported are based upon an initial resin loading of 1.1 mmol/g (extent of labeling indicated by the manufacturer). Unless stated otherwise, all reactions were carried out at room temperature. Starting material was obtained as described in the above Example 2.

### 3.2) Step (1)

A 3 molar solution of 2-azidoethan-1-amine in DMF was added (1 mL per 100 mg of resin) and the vessel was shaken for 45 min, drained and washed with DMF (4 times) and DCM (4 times).

### 3.3) Step (2)

To the resin was added a solution of commercially available 2-[4-(2,5-dioxo-2,5-dihydro-1H-pyrrol-1-yl)phenyl]acetic acid (5 eq), COMU (4.9 eq) and DIPEA (4.9 eq) in DMF (1 mL per 100 mg of resin). The reaction vessel was agitated for 90 min and the resin was washed with DMF (3 times) and DCM (3 times).

### 3.4) Step (3)

Cleavage of the compound of interest from the resin was performed using a 1% TFA in DCM (v/v) solution under agitation for 5 minutes (repeated twice). Resin was filtered and volatiles were removed under reduced pressure to afford a solid crude that was purified using the protocol described in the above Example 2.

### 3.5) Step (4)

The bromoacetylation step was performed by adding 10 eq of bromoacetic acid and 13 eq of diisopropylcarbodiimide in DMF (2 mL per 100 mg of resin). The mixture was agitated for 30 min, drained and washed with DMF (4 times). For the amine displacement step, a 30% (wt) ammonia in water solution was added (2 mL per 100 mg of resin) and the vessel was shaken for 30 min, drained and washed with DMF (4 times) and DCM (4 times). At this stage, compound was cleaved from the resin (as described in step (3)) and purified using the protocol described in the above Example 2.

### 3.6) Step (5)

The ultimate bromoacetylation step was performed by adding 10 eq of bromoacetic acid and 13 eq of diisopropylcarbodiimide in DMF (2 mL per 100 mg of resin). The mixture was agitated for 30 min, drained and washed with DMF (4 times) and DCM (4 times). At this stage, compound was cleaved from the resin (as described in step (3)) and purified using the protocol described in the above Example 2.

### 3.7) Step (6)

The bromoacetylation step was performed by adding 10 eq of bromoacetic acid and 13 eq of diisopropylcarbodiimide in DMF (2 mL per 100 mg of resin). The mixture was agitated for 30 min, drained and washed with DMF (4 times). For the amine displacement step, a 40% (wt) methylamine in water solution was added (1.5 mL per 100 mg of resin) and the vessel was shaken for 30 min, drained and washed with DMF (4 times) and DCM (4 times).

### 3.8) Step (7)

The bromoacetylation step was performed by adding 10 eq of bromoacetic acid and 13 eq of diisopropylcarbodiimide in DMF (2 mL per 100 mg of resin). The mixture was agitated for 30 min, drained and washed with DMF (4 times). For the amine displacement step, a 3 molar solution of 2-azidoethan-1-amine in DMF was added (1 mL per 100 mg of resin) and the vessel was shaken for 45 min, drained and washed with DMF (4 times) and DCM (4 times).

### 3.9) Step (8)

To the resin was added a solution of commercially available 2-[4-(2,5-dioxo-2,5-dihydro-1H-pyrrol-1-yl)phenyl]acetic acid (5 eq), COMU (4.9 eq) and DIPEA (4.9 eq) in DMF (1 mL per 100 mg of resin). The reaction vessel was agitated for 90 min and the resin was washed with DMF (3 times) and DCM (3 times). At this stage, compound was cleaved from the resin (as described in step (3)) and purified using the protocol described in the above Example 2.

### 3.10) Single Molecular Weight Polysarcosine Compounds

The following table 3 lists the resulting PSAR compounds.

TABLE 3

| | | | | HPLC |
|---|---|---|---|---|
| | | | | Method 4 |
| Compound | | | | retention |
| Name | Structure | Yield | MS (ESI⁺) | time |
| PSAR12-N₃- bromoacetamide (white solid) | 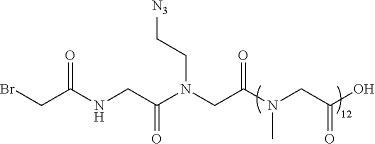 | 65% | Calc [M + 2Na]²⁺ = 609.7155 Exp [M + 2Na]²⁺ = 609.7145 Error = 1.7 ppm | 5.5 min (HPLC Method 3) |
| PSAR12- N₃—NH₂ (white solid) | 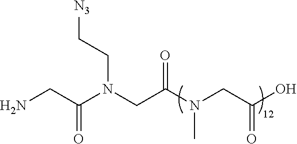 | 69% | Calc [M + H]⁺ = 1054.5 Exp [M + H]⁺ = 1054.5 | 5.1 min (HPLC Method 3) |
| PSAR6-N₃- phenyl-MAL (amorphous beige solid) | 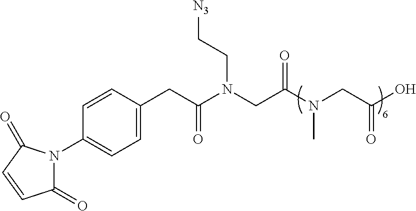 | 81% | Calc [M + H]⁺ = 784.3373 Exp [M + H]⁺ = 784.3342 Error = 3.9 ppm | 18.1 min |
| PSAR12-N₃- phenyl-MAL (beige solid) | 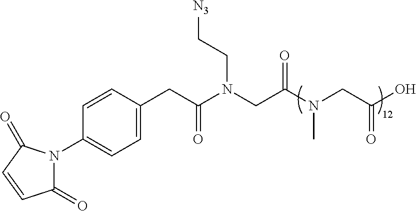 | 74% | Calc [M + 2H]²⁺ = 605.7836 Exp [M + 2H]²⁺ = 605.7833 Error = 0.5 ppm | 17.2 min |
| PSAR18-N₃- phenyl-MAL (beige solid) | 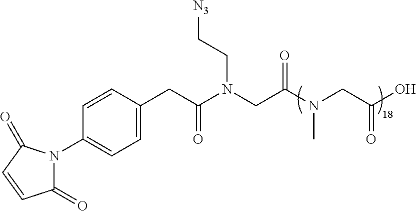 | 62% | Calc [M + 2H]²⁺ = 818.8950 Exp [M + 2H]²⁺ = 818.8957 Error = −0.9 ppm | 16.4 min |
| PSAR24-N₃- phenyl-MAL (beige solid) | 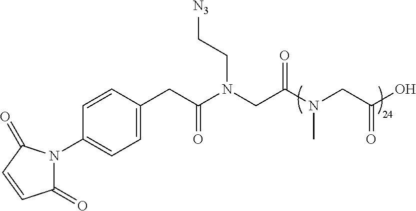 | 60% | Calc [M + 3H]³⁺ = 688.3400 Exp [M + 3H]³⁺ = 688.3415 Error = −2.3 ppm | 16.5 min |
| PSAR18- N₃—N₃- phenyl-MAL (beige solid) | 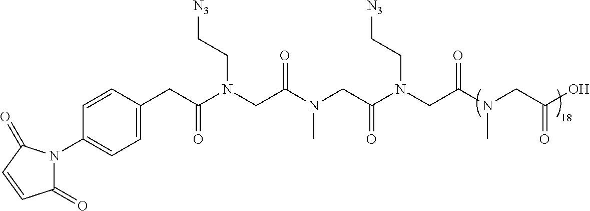 | 62% | Calc [M + 2H]²⁺ = 917.4406 Exp [M + 2H]²⁺ = 917.4438 Error = −3.5 ppm | 18.8 min |

### Example 4: Synthesis of Polysarcosine Compounds Bearing a Terminal Non-Orthogonal Azido-Functionalized Connector (On-Resin Synthesis Method 4)

The reaction scheme is mentioned below.

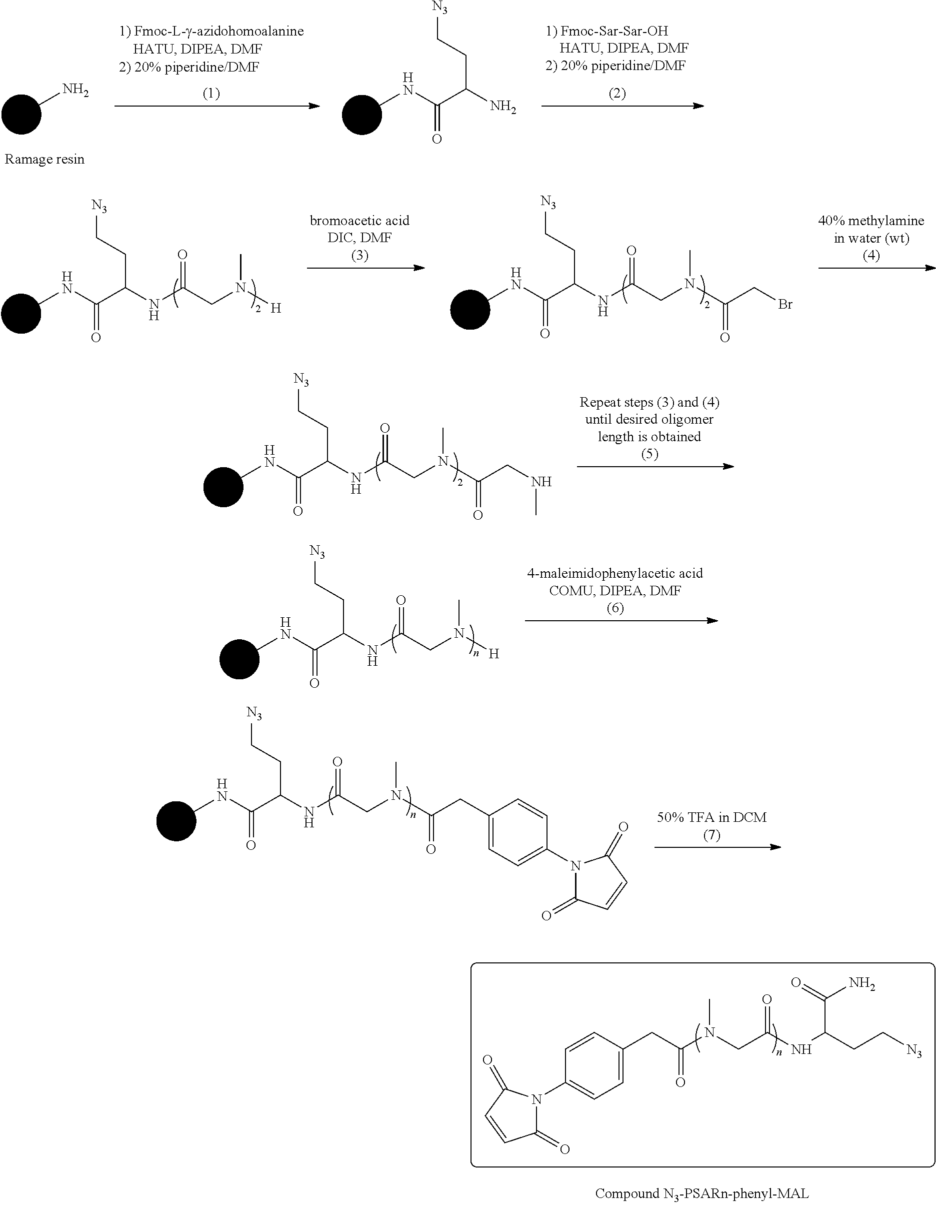

### 4.1) General Methods

On-resin synthesis was performed in empty SPE plastic tubes equipped with a 20 μm polyethylene frit (Sigma-Aldrich). A Titramax 101 platform shaker (Heidolph) was used for agitation. All synthesis yields reported are based upon an initial theoretical resin loading of 0.47 mmol/g (extent of labeling indicated by the manufacturer). Unless stated otherwise, all reactions were carried out at room temperature.

### 4.2) Resin Loading

Typically, 500 mg of Ramage ChemMatrix® beads (0.47 mmol/g, Sigma-Aldrich) were swollen in 5 mL of DCM for 15 min. Resin was treated with 20% piperidine in DMF (1 mL per 100 mg of resin) for 2 times 15 min at room temperature. The resin was then washed with DMF (4 times) and DCM (4 times). To the resin was added a solution of Fmoc-L-γ-azidohomoalanine-OH (3 eq), HATU (2.9 eq) and DIPEA (6 eq) in DMF (1 mL per 100 mg of resin). The reaction vessel was agitated for 1.5 hours and the resin was extensively washed with DMF (5 times) and DCM (5 times). Unreacted sites were acetylated using a capping solution made of acetic anhydride/DIPEA/DMF (1:2:3 v/v) (vessel shaken for 30 min). The solution was drained and the resin was washed with DMF (4 times) and DCM (4 times). Resin was treated with 20% piperidine in DMF (1 mL per 100 mg of resin) for 2 times 15 min at room temperature. The resin was then washed with DMF (4 times) and DCM (4 times).

### 4.3) Fmoc-Sar-Sar-OH Coupling Procedure

To the resin was added a solution of Fmoc-Sar-Sar-OH (4 eq), HATU (3.9 eq) and DIPEA (8 eq) in DMF (1 mL per 100 mg of resin). The reaction vessel was agitated for 2 hours and the resin was extensively washed with DMF (4 times) and DCM (4 times). The resin was treated with 20% piperidine in DMF (1 mL per 100 mg of resin) for 2 times 15 min at room temperature. The resin was then washed with DMF (4 times) and DCM (4 times).

### 4.4) Elongation of Polysarcosine Compounds

Elongation of the polysarcosine oligomer was performed until the desired length was obtained, by alternating bromoacetylation and amine displacement steps. The bromoacetylation step was performed by adding 10 eq of bromoacetic acid and 13 eq of diisopropylcarbodiimide in DMF (2 mL per 100 mg of resin). The mixture was agitated for 30 min, drained and washed with DMF (4 times). For the amine displacement step, a 40% (wt) methylamine in water solution was added (1.5 mL per 100 mg of resin) and the vessel was shaken for 30 min, drained and washed with DMF (4 times) and DCM (4 times).

### 4.5) Step (6)

To the resin was added a solution of commercially available 2-[4-(2,5-dioxo-2,5-dihydro-1H-pyrrol-1-yl)phenyl]acetic acid (5 eq), COMU (4.9 eq) and DIPEA (4.9 eq) in DMF (1 mL per 100 mg of resin). The reaction vessel was agitated for 90 min and the resin was washed with DMF (3 times) and DCM (3 times).

### 4.5) Cleavage from the Resin and Purification

Cleavage of the oligomer from the resin was performed using 5 mL of a TFA/DCM (50:50) solution for 30 minutes under agitation at room temperature. The process was repeated once and the pooled filtrates were evaporated under reduced pressure to give a solid crude that was purified as described in the above Example 2.

### 4.6) Single Molecular Weight Polysarcosine Compounds

The following table 4 lists the resulting PSAR compounds.

TABLE 4

| | | | | HPLC |
|---|---|---|---|---|
| | | | | Method |
| | | | | 4 |
| | | | | retention |
| Compound Name | Structure | Yield | MS (ESI⁺) | time |
| N₃-PSAR12-phenyl- MAL (beige solid) | 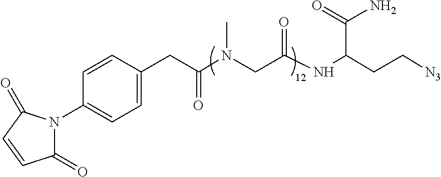 | 82% | Calc [M + 2H]²⁺ = 605.2916 Exp [M + 2H]²⁺ = 605.2936 Error = −3.2 ppm | 12.5 min |

### Example 5: Synthesis of Polyethylene Glycol (PEG) Compounds Bearing an Azido-Functionalized Orthogonal Connector (On-Resin Synthesis Method 5)

The reaction scheme is mentioned below.

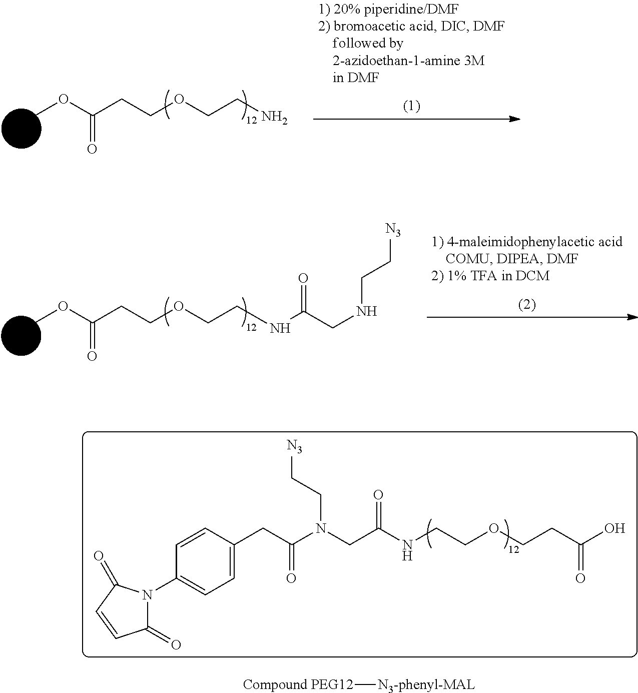

### 5.1) General Methods

On-resin synthesis was performed in empty SPE plastic tubes equipped with a 20 μm polyethylene frit (Sigma-Aldrich). A Titramax 101 platform shaker (Heidolph) was used for agitation. All synthesis yields reported are based upon an initial resin loading of 1.1 mmol/g (extent of labeling indicated by the manufacturer). Unless stated otherwise, all reactions were carried out at room temperature.

### 5.2) Resin Loading

Typically, 200 mg of 2-chlorotrityl chloride resin beads (100-200 mesh, 1% DVB, 1.1 mmol/g, Novabiochem) were swollen in 4 mL of DCM for 10 min. Fmoc-PEG₁₂-CH₂CH₂COOH (PurePEG™, 1.2 eq), previously dissolved in 2 mL of dry DCM, was added onto the resin. DIPEA (3 eq) was added and the reaction vessel was agitated for 1 hour at room temperature. 300 μL of MeOH was added to quench unreacted resin. After 10 min of shaking, the solution was drained and the resin was washed with DMF (3 times) and DCM (3 times). The resin was dried under high vacuum.

### 5.2) Step (1)

The resin was treated with 20% piperidine in DMF (1 mL per 100 mg of resin) for 2 times 15 min at room temperature. The resin was then washed with DMF (4 times) and DCM (4 times). The bromoacetylation step was performed by adding 10 eq of bromoacetic acid and 13 eq of diisopropylcarbodiimide in DMF (2 mL per 100 mg of resin). The mixture was agitated for 30 min, drained and washed with DMF (4 times). For the amine displacement step, a 3 molar solution of 2-azidoethan-1-amine in DMF was added (1 mL per 100 mg of resin) and the vessel was shaken for 45 min, drained and washed with DMF (4 times) and DCM (4 times).

### 5.3) Step (2)

The 2-[4-(2,5-dioxo-2,5-dihydro-1H-pyrrol-1-yl)phenyl]acetic acid coupling procedure and the cleavage from the resin was performed as described in the above Example 3 and the purification of the compound was performed as described in the above Example 2.

### 5.4) Single Molecular Weight PEG Compounds

The following table 5 lists the resulting PEG compounds.

TABLE 5

| | | | | HPLC |
|---|---|---|---|---|
| | | | | Method |
| | | | | 4 |
| Compound | | | MS | retention |
| Name | Structure | Yield | (ESI⁺) | time |
| PEG12-N₃- phenyl- MAL (slightly yellow oil) | 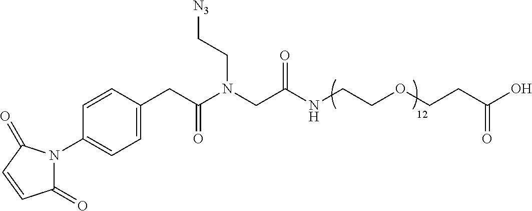 | 33% | Observed [M + H]⁺ = 957.5 | 18.1 min |

### Example 6: Synthesis of MMAE, SN38, Exatecan and PNU159682-Based Intermediate Compounds

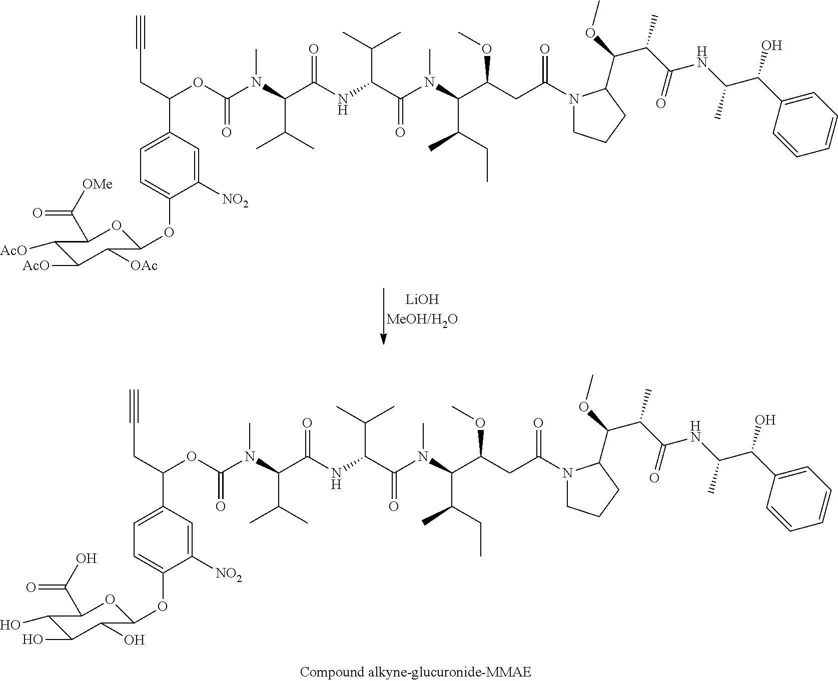 [SMILES: C#CCC(OC(=O)N(C)[C@@H](C(=O)N[C@@H](C(=O)N(C)[C@H]([C@H](C)CC)[C@H](CC(=O)N1CCCC1[C@@H](OC)[C@H](C)C(=O)N[C@@H](C)[C@H](O)c1ccccc1)OC)C(C)C)C(C)C)c1ccc(OC2OC(C(=O)OC)C(OC(C)=O)C(OC(C)=O)C2OC(C)=O)c([N+](=O)[O-])c1 ; C#CCC(OC(=O)N(C)[C@@H](C(=O)N[C@@H](C(=O)N(C)[C@H]([C@H](C)CC)[C@H](CC(=O)N1CCCC1[C@@H](OC)[C@H](C)C(=O)N[C@@H](C)[C@H](O)c1ccccc1)OC)C(C)C)C(C)C)c1ccc(OC2OC(C(=O)O)C(O)C(O)C2O)c([N+](=O)[O-])c1]

110.8 mg (0.087 mmol) of starting material (synthesized as described in Renoux et al., Chem. Sci., 2017, 8(5), 3427-3433) was dissolved in MeOH (10 mL) at 0° C. LiOH monohydrate (36.7 mg/0.87 mmol) was dissolved in water (1 mL) and was slowly added to the reaction vessel. After stirring at 0° C. for 70 min, the mixture was neutralized with acetic acid (68.2 mg/1.14 mmol) and concentrated under reduced pressure. The resulting material was taken up in a water/MeOH/DMF solution (1:1:1 v/v) and purified on a 30 g Biotage® SNAP Ultra C18 (25 μm) cartridge. Mobile phase A was water+0.05% TFA and mobile phase B was acetonitrile+0.05% TFA. The gradient ranged from 10 to 60% B.

Compound alkyne-glucuronide-MMAE (mixture of two diastereoisomers) was obtained as a white solid (95 mg/96%). LC-HRMS m/z (ESI+): Calc [M+H]⁺=1127.5758; Exp [M+H]⁺=1127.5757; Error=0.1 ppm. HPLC Method 3 retention time=10.3 min.

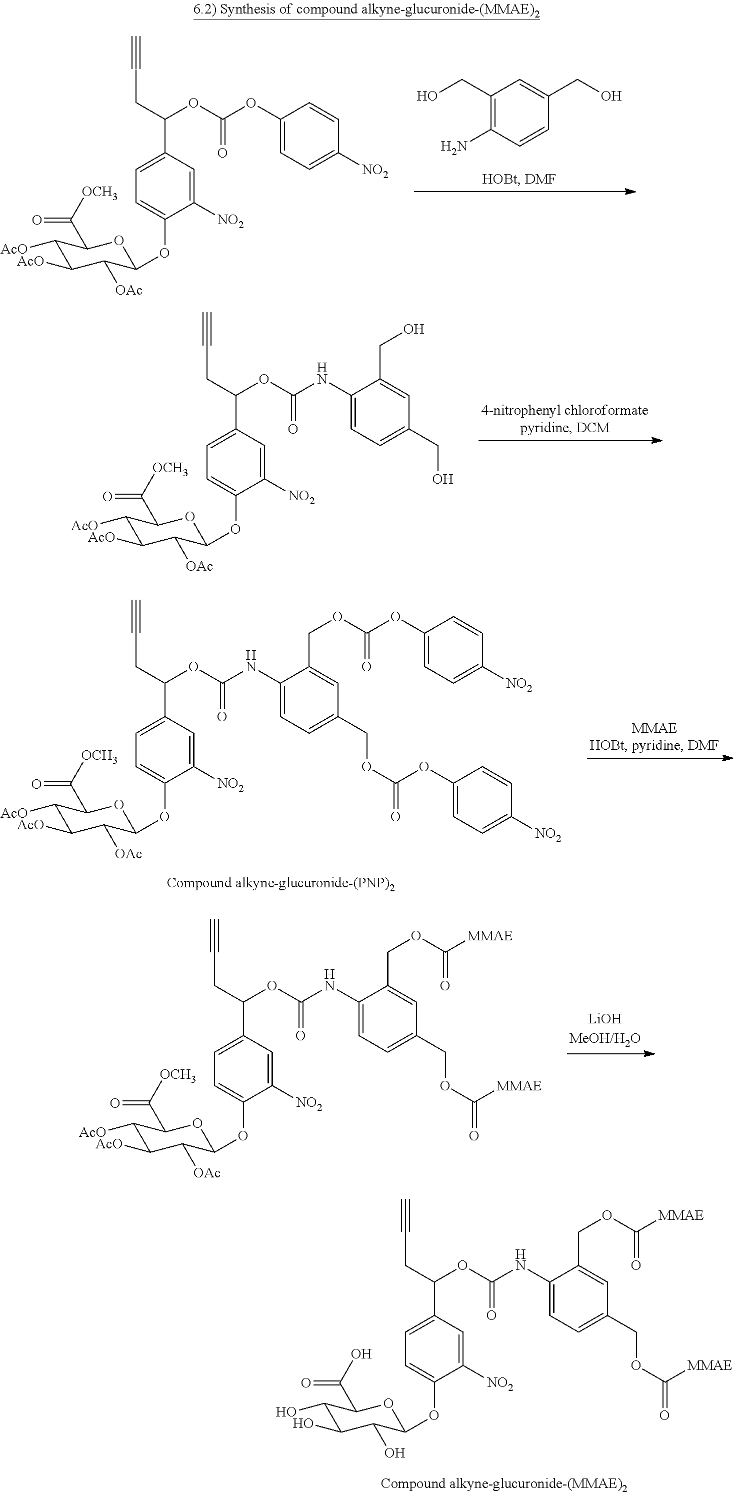

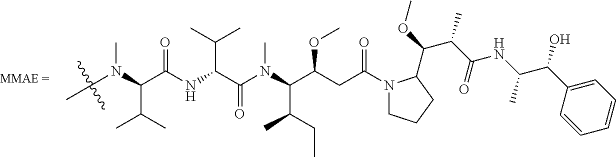 [SMILES: *N(C)[C@@H](C(=O)N[C@@H](C(=O)N(C)[C@H]([C@H](C)CC)[C@H](CC(=O)N1CCCC1[C@@H](OC)[C@H](C)C(=O)N[C@@H](C)[C@H](O)c1ccccc1)OC)C(C)C)C(C)C]

### 6.2.1) Synthesis of Compound Alkyne-Glucuronide-(PNP)₂

165 mg (0.240 mmol) of starting material (synthesized as described in Renoux et al., Chem. Sci., 2017, 8(5), 3427-3433), 64.2 mg (0.419 mmol) of commercially available 4-amino-3-(hydroxymethyl)phenylmethanol and 40.7 mg (0.300 mmol) of HOBt were dissolved in anhydrous DMF. After stirring at 50° C. for 3 hours, the volatiles were evaporated and the residue was purified by chromatography on silica gel (petroleum ether/EtOAc, gradient from 40:60 to 0:100) to afford the intermediate diol compound as a yellow foam.

Anhydrous pyridine (4 molar equivalent) was added dropwise to a cooled solution (0° C.) of 4-nitrophenyl chloroformate (4 molar equivalent) in anhydrous DCM. The mixture was stirred 15 min at 0° C. A solution of the previous intermediate diol compound (1 molar equivalent) in DCM was added and the mixture was stirred 1 hour at room temperature. The reaction was quenched with a saturated solution of NaCl and was extracted 3 times with DCM. The organic phase was dried over MgSO₄, filtered and evaporated under vacuum to afford a solid crude that was purified by chromatography on silica gel (petroleum ether/EtOAc, gradient from 60:40 to 30:70) to afford compound alkyne-glucuronide-(PNP)₂ (52 mg/21% over two steps) as a white solid. ¹H NMR (300 MHz, CDCl₃) δ (ppm) 2.04 (s, 3H), 2.06 (s, 3H), 2.11 (d, J=2.0 Hz, 3H), 2.71-2.92 (m, 2H), 3.72 (s, 3H), 4.11 (q, J=7.1 Hz, 1H), 4.22 (d, J=8.6 Hz, 1H), 5.18-5.41 (m, 8H), 5.87 (t, J=6.5 Hz, 1H), 7.31-7.41 (m, 5H), 7.45-7.55 (m, 2H), 7.56-7.71 (m, 2H), 7.83 (d, J=8.2 Hz, 1H), 7.89 (s, 1H), 8.21-8.32 (m, 4H). HRMS m/z (ESI⁺): Calc [M+Na]⁺=1055.1925; Exp [M+Na]⁺=1055.1955; Error=−2.9 ppm.

### 6.2.2) Synthesis of Compound Alkyne-Glucuronide-(MMAE)₂

52 mg (0.050 mmol) of previous compound alkyne-glucuronide-(PNP)₂, 13.7 mg (0.100 mmol) of HOBt and 74.1 mg (0.103 mmol) of monomethyl auristatin E (MMAE) were dissolved in 1 mL of a 8:2 (v/v) mixture of anhydrous DMF/pyridine. The reaction was stirred 24 hours at room temperature and volatiles were evaporated under reduced pressure. The crude residue was purified by chromatography on silica gel (DCM/MeOH gradient from 97:3 to 90:10) to afford 77 mg (70%) of intermediate compound that was directly engaged into the deprotection step without extensive characterization. 77 mg (0.035 mmol) of this compound was dissolved in MeOH (7 mL) at 0° C. LiGH monohydrate (14.7 mg/0.350 mmol) was dissolved in water (0.7 mL) and was slowly added to the reaction vessel. After stirring at 0° C. for 60 min, the mixture was neutralized with acetic acid (27.4 mg/0.457 mmol) and concentrated under reduced pressure. The resulting material was taken up in a water/MeOH/DMF solution (1:1:1 v/v) and purified on a 30 g Biotage® SNAP Ultra C18 (25 μm) cartridge. Mobile phase A was water+0.05% TFA and mobile phase B was acetonitrile+0.05% TFA.

Compound alkyne-glucuronide-(MMAE)₂ (mixture of two diastereoisomers) was obtained as a white solid (40 mg/56%). LC-HRMS m/z (ESI⁺): Calc [M+2H]²⁺=1025.5623; Exp [M+2H]²⁺=1025.5599; Error=2.4 ppm. HPLC Method 3 retention time=13.0 min.

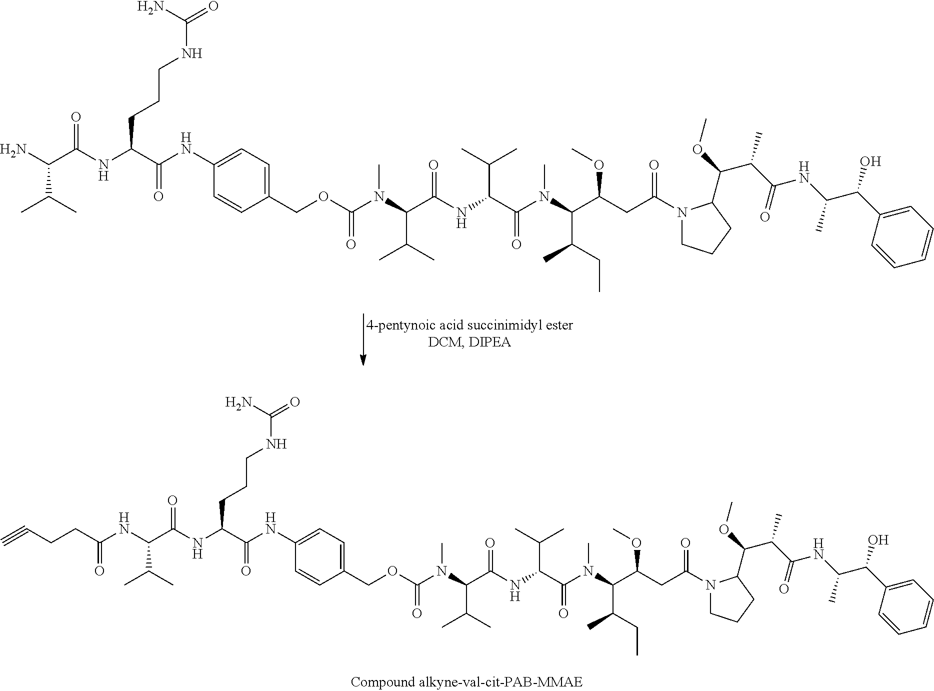 [SMILES: CC[C@@H](C)[C@H]([C@H](CC(=O)N1CCCC1[C@@H](OC)[C@H](C)C(=O)N[C@@H](C)[C@H](O)c1ccccc1)OC)N(C)C(=O)[C@H](NC(=O)[C@@H](C(C)C)N(C)C(=O)OCc1ccc(NC(=O)[C@H](CCCNC(N)=O)NC(=O)[C@@H](N)C(C)C)cc1)C(C)C ; C#CCCC(=O)N[C@H](C(=O)N[C@@H](CCCNC(N)=O)C(=O)Nc1ccc(COC(=O)N(C)[C@@H](C(=O)N[C@@H](C(=O)N(C)[C@H]([C@H](C)CC)[C@H](CC(=O)N2CCCC2[C@@H](OC)[C@H](C)C(=O)N[C@@H](C)[C@H](O)c2ccccc2)OC)C(C)C)C(C)C)cc1)C(C)C]

58 mg (0.052 mmol) of starting material (synthesized as described in Tang et al., Org. Biomol. Chem., 2016,14(40), 9501-9518) and 15 mg (0.077 mmol) of 4-pentynoic acid succinimidyl ester were dissolved in 3 mL of anhydrous DCM. 16.7 mg (0.129 mmol) of DIPEA was added and the reaction was stirred 16 hours at room temperature under and argon atmosphere. Volatiles were then removed under vacuum, the resulting material was taken up in a DMF solution and was purified on a 30 g Biotage® SNAP Ultra C18 (25 μm) cartridge. Mobile phase A was water+0.1% TFA and mobile phase B was acetonitrile+0.1% TFA. The gradient ranged from 25 to 70% B.

Compound alkyne-val-cit-PAB-MMAE was obtained as a white solid (21 mg/34%). ESI⁺[M+Na]⁺=1225.7. HPLC Method 3 retention time=9.0 min.

### 6.4 Synthesis of Compound Alkyne-SN38

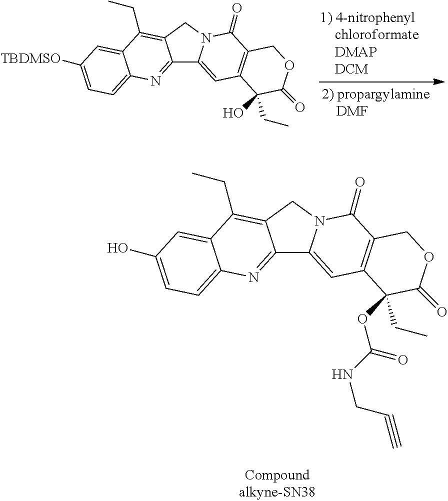 [SMILES: CCc1c2c(nc3ccc(O[Si](C)(C)C(C)(C)C)cc13)-c1cc3c(c(=O)n1C2)COC(=O)[C@]3(O)CC ; C#CCNC(=O)O[C@]1(CC)C(=O)OCc2c1cc1n(c2=O)Cc2c-1nc1ccc(O)cc1c2CC]

156 mg (0.308 mmol) of starting material TBDMS-SN38 (synthesized as described in Moon et al., J. Med. Chem., 2008, 51(21), 6916-6926), 113 mg (0.924 mmol) of 4-(dimethylamino)pyridine and 75 mg (0.369 mmol) of 4-nitrophenyl chloroformate were dissolved in 8 mL of anhydrous DCM. The solution was stirred at room temperature for 90 min, was diluted with 5% acetic acid in water and was extracted 3 times with DCM. The organic phase was dried over MgSO₄, filtered and evaporated under vacuum to afford a yellow solid that was engaged in the next step without further purification.

175 mg (0.261 mmol) of this yellow solid was dissolved in 4 mL of anhydrous DMF and 43 mg (0.782 mmol) of propargylamine was slowly added. The reaction was stirred at room temp for 16 hours under an argon atmosphere. Volatiles were removed under vacuum and the residue was purified by chromatography on silica gel (petroleum ether/EtOAc, gradient from 40:60 to 0:100) to afford compound alkyne-SN38 (71 mg/56%) as a bright yellow solid. HRMS m/z (ESI⁺): Calc [M+H]⁺=474.1660; Exp [M+H]⁺=474.1664; Error=−0.9 ppm. HPLC Method 3 retention time=9.7 min. TLC eluting with 100% EtOAc: Rf=0.15.

### 6.5 Synthesis of Compound Alkyne-Glucuronide-SN38

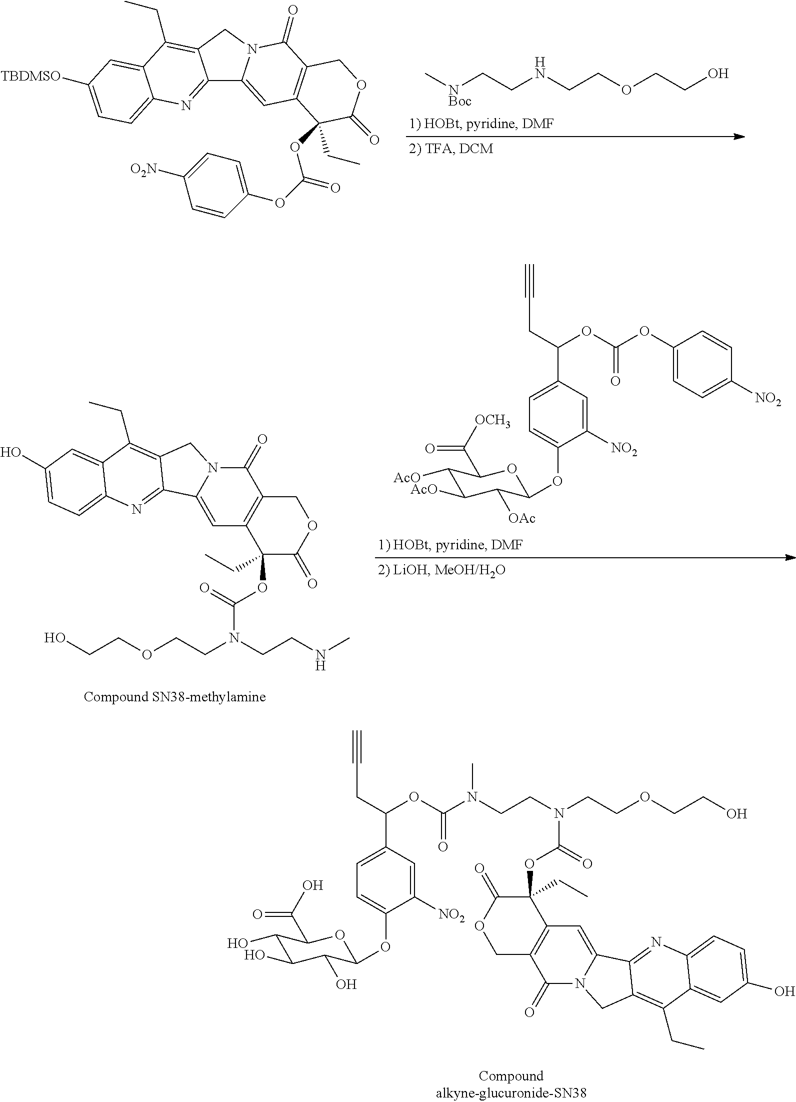 [SMILES: CCc1c2c(nc3ccc(O[Si](C)(C)C(C)(C)C)cc13)-c1cc3c(c(=O)n1C2)COC(=O)[C@@]3(CC)OC(=O)Oc1ccc([N+](=O)[O-])cc1 ; CN(CCNCCOCCO)C(=O)OC(C)(C)C ; CCc1c2c(nc3ccc(O)cc13)-c1cc3c(c(=O)n1C2)COC(=O)[C@@]3(CC)OC(=O)N(CCNC)CCOCCO ; C#CCC(OC(=O)Oc1ccc([N+](=O)[O-])cc1)c1ccc(OC2OC(C(=O)OC)C(OC(C)=O)C(OC(C)=O)C2OC(C)=O)c([N+](=O)[O-])c1 ; C#CCC(OC(=O)N(C)CCN(CCOCCO)C(=O)O[C@]1(CC)C(=O)OCc2c1cc1n(c2=O)Cc2c-1nc1ccc(O)cc1c2CC)c1ccc(OC2OC(C(=O)O)C(O)C(O)C2O)c([N+](=O)[O-])c1]

50 mg (0.074 mmol) of starting material TBDMS-SN38-OPNP (synthesized as described in the previous section 6.4) and 5 mg (0.037 mmol) of HOBt were weighted in a reaction vessel. 58.5 (0.223 mmol) of tert-butyl (2-((2-(2-hydroxyethoxy)ethyl)amino)ethyl)(methyl)carbamate (synthesized as described in WO2011/133039), previously dissolved in 1 mL of a 8:2 (v/v) mixture of anhydrous DMF/pyridine, was added into the reaction vessel. The reaction was stirred 16 hours at room temperature and volatiles were evaporated under reduced pressure. The crude residue was purified by chromatography on silica gel (DCM/MeOH gradient from 98:2 to 90:10) to afford 48 mg (95%) of intermediate compound (yellow solid) that was directly engaged into the deprotection step. HRMS m/z (ESI⁺): Calc [M+H]⁺=681.3130; Exp [M+H]⁺=681.3113; Error=2.5 ppm.

48 mg (0.071 mmol) of this compound was dissolved in 2 mL of DCM and 500 μL of TFA was added. The solution was stirred 90 min at room temperature and volatiles were removed under reduced pressure. The crude residue was purified by chromatography on silica gel (DCM/MeOH gradient from 94:6 to 80:20) to afford 39.8 mg (96%) of compound SN38-methylamine as a yellow solid. HRMS m/z (ESI⁺): Calc [M+H]⁺=581.2606; Exp [M+H]⁺=581.2601; Error=0.8 ppm. HPLC Method 3 retention time=7.7 min.

48 mg (0.069 mmol) of starting material (synthesized as described in Renoux et al., Chem. Sci., 2017, 8(5), 3427-3433), 39.8 mg (0.069 mmol) of previous compound SN38-methylamine and 9.3 mg (0.069 mmol) of HOBt were dissolved with 1.5 mL of a 8:2 (v/v) mixture of anhydrous DMF/pyridine. The reaction was stirred 16 hours at room temperature and volatiles were evaporated under reduced pressure. The crude residue was purified by chromatography on silica gel (DCM/MeOH gradient from 98:2 to 95:5) to afford 57 mg (74%) of intermediate compound (yellow solid) that was directly engaged into the deprotection step. ESI⁺[M+H]⁺=1130.4.

57 mg (0.050 mmol) of this compound was dissolved in MeOH (6 mL) at 0° C. LiOH monohydrate (21.2 mg/0.504 mmol) was dissolved in water (0.6 mL) and was slowly added to the reaction vessel. After stirring at 0° C. for 70 min, the mixture was neutralized with acetic acid (39.4 mg/0.656 mmol) and concentrated under reduced pressure. The resulting material was taken up in a water/MeOH/DMF solution (1:1:1 v/v) and purified on a 30 g Biotage® SNAP Ultra C18 (25 μm) cartridge. Mobile phase A was water+0.05% TFA and mobile phase B was acetonitrile+0.05% TFA. The gradient ranged from 10 to 60% B.

Compound alkyne-glucuronide-SN38 was obtained as a yellow solid (20.5 mg/42%). LC-HRMS m/z (ESI⁺): Calc [M+H]⁺=990.3251; Exp [M+H]⁺=990.3210; Error=4.1 ppm. HPLC Method 3 retention time=8.2 min.

### 6.6 Synthesis of compound alkyne-glucuronide-Exatecan

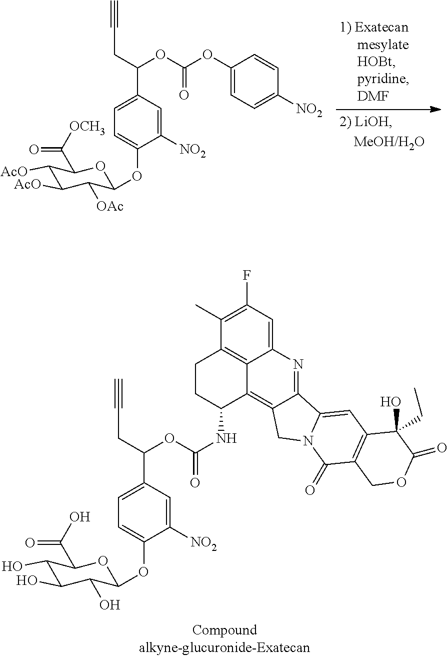 [SMILES: C#CCC(OC(=O)Oc1ccc([N+](=O)[O-])cc1)c1ccc(OC2OC(C(=O)OC)C(OC(C)=O)C(OC(C)=O)C2OC(C)=O)c([N+](=O)[O-])c1 ; C#CCC(OC(=O)N[C@@H]1CCc2c(C)c(F)cc3nc4c(c1c23)Cn1c-4cc2c(c1=O)COC(=O)[C@@]2(O)CC)c1ccc(OC2OC(C(=O)O)C(O)C(O)C2O)c([N+](=O)[O-])c1]

132.1 mg (0.192 mmol) of starting material (synthesized as described in Renoux et al., Chem. Sci., 2017, 8(5), 3427-3433), 102 mg (0.192 mmol) of Exatecan mesylate and 26 mg (0.192 mmol) of HOBt were dissolved with 1 mL of a 8:2 (v/v) mixture of anhydrous DMF/pyridine. The reaction was stirred 16 hours at room temperature and volatiles were evaporated under reduced pressure. The crude residue was purified by chromatography on silica gel (DCM/MeOH gradient from 98:2 to 90:10) to afford 165 mg (87%) of intermediate compound (yellow solid) that was directly engaged into the deprotection step. ESI [M+H]⁺=985.3.

165 mg (0.168 mmol) of this compound was dissolved in MeOH/THF 1:1 v/v (16 mL) at 0° C. LiOH monohydrate (70.3 mg/1.675 mmol) was dissolved in water (1.6 mL) and was slowly added to the reaction vessel. After stirring at 0° C. for 70 min, the mixture was neutralized with acetic acid (131 mg/2.18 mmol) and concentrated under reduced pressure. The resulting material was taken up in a water/MeOH/DMF solution (1:1:1 v/v) and purified on a 30 g Biotage® SNAP Ultra C₁₈ (25 μm) cartridge. Mobile phase A was water+0.05% TFA and mobile phase B was acetonitrile+0.05% TFA. The gradient ranged from 10 to 50% B.

Compound alkyne-glucuronide-Exatecan was obtained as a yellow solid (98 mg/69%). LC-HRMS m/z (ESI⁺): Calc [M+H]⁺=845.2312; Exp [M+H]⁺=845.2360 Error=−4.8 ppm. HPLC Method 3 retention time=8.0 min.

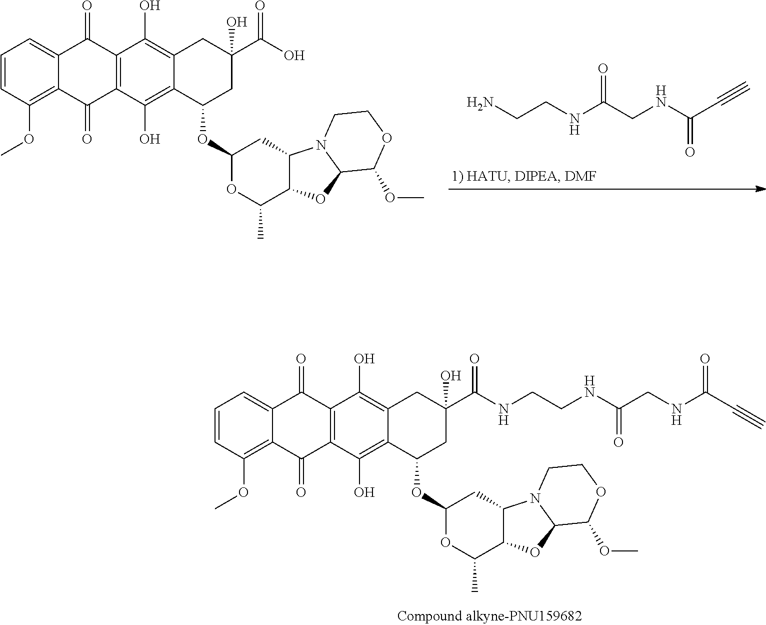 [SMILES: COc1cccc2c1C(=O)c1c(O)c3c(c(O)c1C2=O)C[C@@](O)(C(=O)O)C[C@@H]3O[C@H]1C[C@H]2[C@H](O[C@@H]3[C@@H](OC)OCCN32)[C@H](C)O1 ; C#CC(=O)NCC(=O)NCCN ; C#CC(=O)NCC(=O)NCCNC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O]

762 mg (4.54 mmol) of glycine tert-butyl ester hydrochloride, 318.3 mg (4.54 mmol) of propiolic acid and 61.4 mg (0.454 mmol) of HOBt were dissolved in 5 mL of anhydrous DMF. 1103 mg (10.9 mmol) of DIPEA was added and the solution was stirred on ice (0° C.) for 10 min. 871 mg (4.54 mmol) of EDC hydrochloride was suspended in 12 mL of anhydrous DMF and added into the reaction vessel. The mixture was stirred 16 hours at room temperature in the dark. Volatiles were then removed under reduced pressure. A saturated solution of NH₄Cl was added and was extracted 3 times with DCM. The organic phase was dried over MgSO₄, filtered and evaporated. The crude residue was purified by chromatography on silica gel (petroleum ether/EtOAc gradient from 80:20 to 50:50) to afford 255 mg (31%) of tert-butyl propioloylglycinate as a transparent oil. MS (ESI⁺): [M+H]⁺=184.0; TLC eluting with petroleum ether/EtOAc (40:60 v/v) and stained with KMnO₄: Rf=0.75.

255 mg (1.39 mmol) of tert-butyl propioloylglycinate was dissolved in 5 mL of a DCM/TFA (1:1 v/v) solution. Deprotection reaction was complete after 1 hour of stirring at room temperature, as assessed by TLC analysis. Volatiles were removed under reduced pressure to yield 188 mg (105%) of propioloylglycine as an oily residue that was engaged in the next step without purification.

178 mg (1.40 mmol) of propioloylglycine and 504.8 mg (1.33 mmol) of HATU were dissolved in 3 mL of anhydrous DMF. 180.6 mg (1.40 mmol) of DIPEA was added and the reaction was stirred 5 min at room temperature. 291 mg (1.82 mmol) of N-Boc-ethylenediamine, previously dissolved in 1 mL of anhydrous DMF, was then added and the reaction mixture was stirred 30 min at room temperature in the dark. Volatiles were then removed under reduced pressure. A saturated solution of NH₄Cl was added and was extracted 3 times with DCM. The organic phase was dried over MgSO₄, filtered and evaporated. The crude residue was purified by chromatography on silica gel (petroleum ether/EtOAc gradient from 20:80 to 0:100) to afford 204 mg (54%) of tert-butyl (2-(2-propiolamidoacetamido)ethyl)carbamate as a slightly yellow oil. MS (ESI⁺): [M+H]⁺=270.1; TLC eluting with 100% EtOAc and stained with KMnO₄: Rf=0.35.

204 mg (0.758 mmol) of tert-butyl (2-(2-propiolamidoacetamido)ethyl)carbamate was taken up in 5 mL of a DCM/TFA (7:3 v/v) solution. Deprotection reaction was complete after 45 min of stirring at room temperature, as assessed by TLC analysis. Volatiles were removed under high vacuum overnight to yield 196 mg (92%) of N-(2-((2-aminoethyl)amino)-2-oxoethyl)propiolamide TFA salt as a slightly yellow thick wax. ¹H NMR (300 MHz, DMSO-d₆) δ (ppm) 2.84 (q, J=6.2 Hz, 2H), 3.29 (q, J=6.3 Hz, 2H), 3.72 (d, J=6.0 Hz, 2H), 4.20 (s, 1H), 7.78 (br. s, 3H), 8.12 (t, J=5.6 Hz, 1H), 8.94 (t, J=5.9 Hz, 1H). MS (ESI⁺): [M+H]⁺=170.0.

### 6.7.2) Synthesis of alkyne-PNU159682

25 mg (0.040 mmol) of PNU159692 carboxylic acid derivative (purple solid synthesized as described in WO/2016/040825, see chemical structure above) and 15.1 mg (0.040 mmol) of HATU were dissolved in 1 mL of anhydrous DMF in a round-bottom flask. 10.2 mg (0.080 mmol) of DIPEA was added and the mixture was stirred for 2 min at room temperature. 13.4 mg (0.047 mmol) of N-(2-((2-aminoethyl)amino)-2-oxoethyl)propiolamide TFA salt (previously dissolved in 500 μL of anhydrous DMF) was added and the reaction mixture was stirred at room temperature for 5 min. Volatiles were removed under high vacuum and the residue was purified by chromatography on silica gel (DCM/MeOH, gradient from 99:1 to 90:10) to afford 14.7 mg (49%) of alkyne-PNU159682 as a red solid. HRMS m/z (ESI⁺): Calc [M+H]⁺=779.2770; Exp [M+H]⁺=779.2758; Error=1.5 ppm. HPLC Method 6 retention time =5.4 min.

### Example 7: Synthesis of Polysarcosine-Based Drug Conjugate Linkers Using Glutamic Acid as Orthogonal Moiety

The reaction scheme is mentioned below.

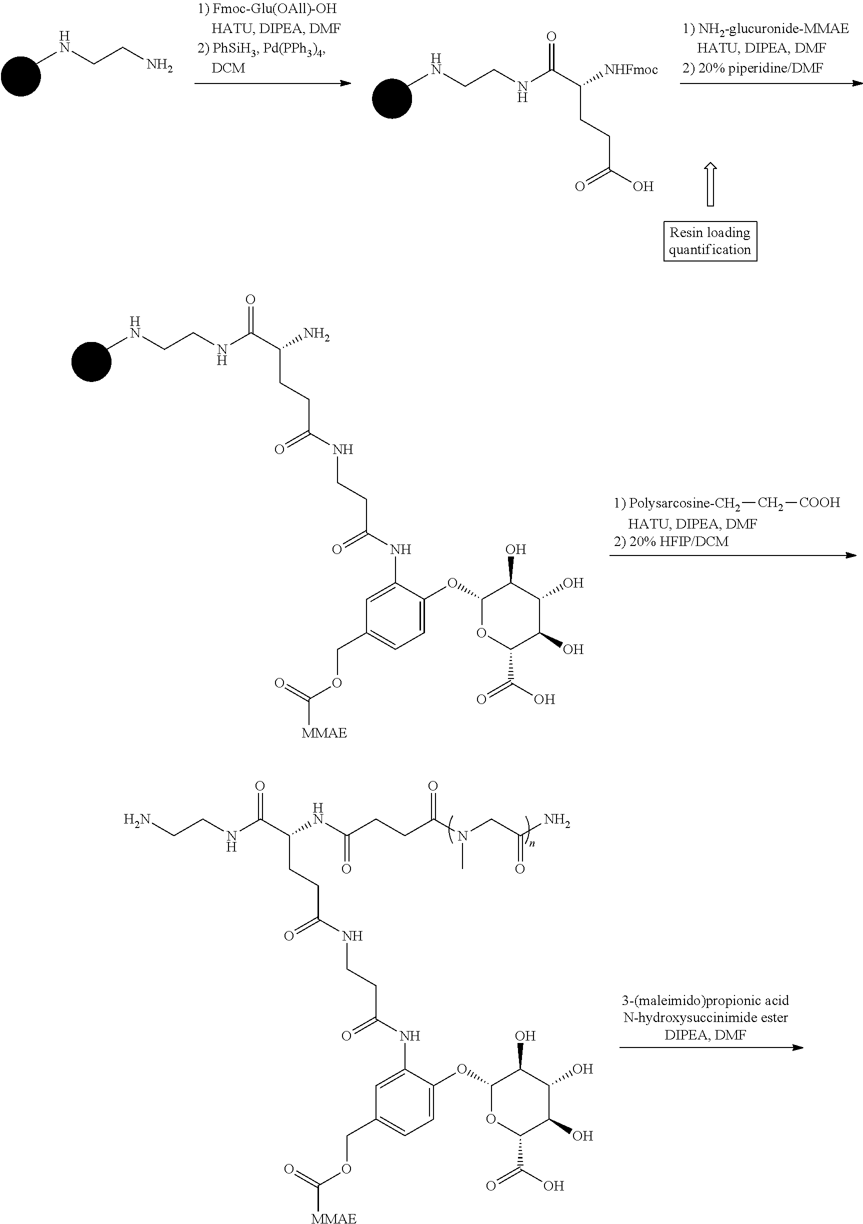

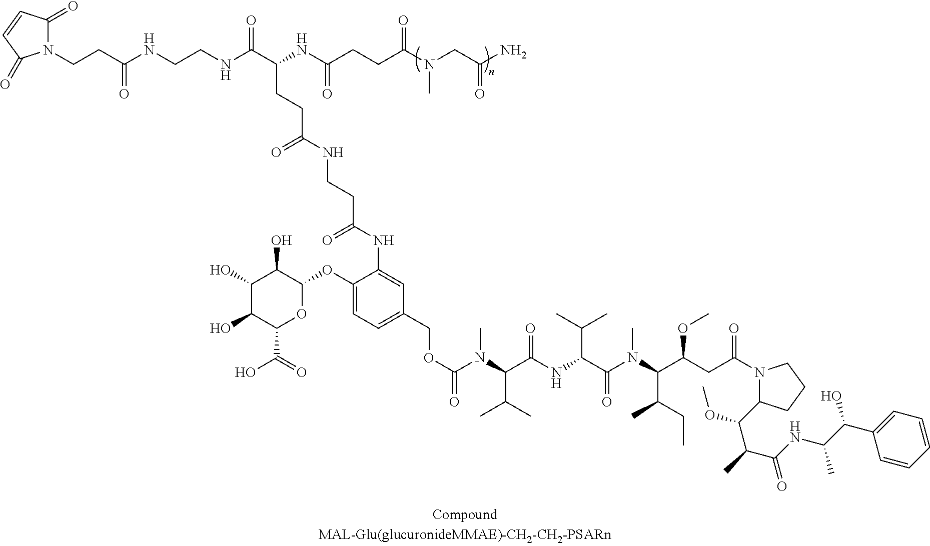

### 7.1) Resin Loading with Ethylenediamine

500 mg of 2-chlorotrityl chloride resin beads (100-200 mesh, 1% DVB, 1.1 mmol/g, 0.55 mmol scale, Novabiochem) were swollen in 5 mL of DCM for 10 min. 5 eq (2.75 mmol, 165.3 mg) of ethylenediamine (Sigma-Aldrich) was added and the mixture was shaken at room temp for 4 hours, followed by extensive washes with DCM (5 times 5 mL). Unreacted sites on the resin were capped using a DCM/MeOH/DIPEA (17:2:1 v/v) solution (20 min treatment). The resin was extensively washed with DCM (5 times 5 mL) and MeOH (5 times 5 mL), dried under vacuum and stored at −20° C. until further use.

### 7.2) Fmoc-Glu(OAll)-OH coupling

To the resin containing deprotected N-terminus (1 eq) was added a solution of Fmoc-Glu(OAll)-OH (3 eq), HATU (2.85 eq) and DIPEA (6 eq) in DMF (1 mL per 100 mg of resin). The reaction vessel was agitated for 2 hours and the resin was extensively washed with DMF (5 times 3 mL) and DCM (5 times 3 mL). Reaction completeness was confirmed by a negative Kaiser test. The resin was dried under vacuum and stored at −20° C. until further use.

### 7.3) Alloc Protecting Group Removal

The resin was suspended in DCM (4 mL per 100 mg of resin) and the mixture was gently agitated by a stream of argon introduced from below the fritted disc. Phenylsilane (20 eq) was added and agitation was continued for 5 min after which Pd(PPh₃)₄ (0.25 eq) was added. Agitation of the mixture at room temperature under the argon stream was continued under protection from light for 30 min after which the solution was drained. Treatment with phenylsilane and Pd(PPh₃)₄ was repeated once and the resin was thoroughly washed with DCM (5 times 5 mL), DMF (5 times 5 mL) and MeOH (5 times 5 mL). The resin was dried under vacuum and stored at −20° C. until further use. Resin loading was assessed (Fmoc cleavage test, absorbance measurement at 301 nm) and was usually 0.70-0.80 mmol/g.

### 7.4) (2R,3R,4R,5S,6R)-6-(2-(3-aminopropanamido)-4-((5S,8S,11S,12R)-11-((S)-sec-butyl)-12-(2-(2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (NH₂-glucuronide-MMAE) coupling procedure

To the resin containing deprotected carboxylic acid group (1 eq) was added a solution of HATU (4 eq) and DIPEA (4.2 eq) in DMF. The reaction vessel was agitated for 25 min, drained and the resin was washed with DMF (4 times 5 mL). To the resin was then added 1.5 eq of compound (2R,3R,4R,5S,6R)-6-(2-(3-aminopropanamido)-4-((5S,8S,11S,12R)-11-((S)-sec-butyl)-12-(2-(2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (“NH₂-glucuronide-MMAE”; synthesized as described in Jeffrey SC et al., Bioconjug. Chem., 2006, 17(3), 831-840) and DIPEA (3.2 eq) in DMF. The reaction vessel was agitated for 3 hours, drained and washed with DMF (5 times 3 mL) and DCM (5 times 3 mL). The resin was dried under vacuum and stored at −20° C. until further use.

### 7.5) Fmoc Deprotection Procedure

Resin containing Fmoc-protected aminoacid was treated with 20% piperidine in DMF (1 mL per 100 mg of resin) for 2 times 15 min at room temperature. The resin was then washed with DMF (5 times 5 mL) and DCM (5 times 5 mL). The resin was dried under vacuum and stored at −20° C. until further use.

### 7.6) Polysarcosine Coupling Procedure

To the resin containing deprotected primary amine group (1 eq) was added a solution of polysarcosine-CH₂—CH₂—COOH (2.2 eq), HATU (2 eq) and DIPEA (6 eq) in DMF. The reaction vessel was agitated for 2.5 hours, drained and the resin was washed with DMF (3 times 5 mL) and DCM (3 times 5 mL). The resin was dried under vacuum and stored at −20° C. until further use.

7.7) Cleavage from Resin

Final cleavage from the 2-chlorotrityl resin was performed at room temperature under agitation using a 20% (v/v) HFIP in DCM solution (2 mL per 100 mg of resin). Reaction time was 60 min. Resin was filtered and the obtained solution was evaporated under a stream of argon gas. The final residue was dried under high vacuum and was used as is in the following step.

### 7.8) 3-(Maleimido)Propionic Acid N-Hydroxysuccinimide Ester Coupling Procedure

To the residue dissolved in anhydrous DMF was added 3-(maleimido)propionic acid N-hydroxysuccinimide ester (8 eq). DIPEA (10 eq) was added and the mixture was agitated at room temperature for 30 min. The reaction mixture was quenched with water/TFA (99.5:0.5 v/v) and purified on a 30 g Biotage® SNAP Ultra C₁₈ (25 μm) cartridge. Mobile phase A was water+0.05% TFA and mobile phase B was acetonitrile+0.05% TFA. The gradient ranged from 10 to 60% B.

Compound MAL-Glu(glucuronideMMAE)-CH₂—CH₂—PSAR6 was obtained as a transparent oil (5.8 mg/14% yield based on initial resin loading). LC-HRMS m/z (ESI⁺): Calc [M+2H]²⁺=989.5064; Exp [M+2H]²⁺989.5023; Error=4.2 ppm. HPLC Method 1 retention time=6.7 min.

Compound MAL-Glu(glucuronideMMAE)-CH₂—CH₂—PSAR12 was obtained as a transparent oil (4.6 mg/20% yield based on initial resin loading). LC-HRMS m/z (ESI⁺): Calc [M+2H]²⁺=1202.6178; Exp [M+2H]²⁺1202.6178; Error=0.0 ppm. HPLC Method 1 retention time=6.9 min.

Compound MAL-Glu(glucuronideMMAE)-CH₂—CH₂—PSAR18 was obtained as a transparent oil (2.4 mg/14% yield based on initial resin loading). LC-HRMS m/z (ESI⁺): Calc [M+2H]²⁺=1415.7291; Exp [M+2H]²⁺1415.7282; Error=0.7 ppm. HPLC Method 1 retention time=6.8 min.

### Example 8: Synthesis of Polysarcosine-Based Drug Conjugate Linkers Using Lysine as Orthogonal Moiety

The reaction scheme is mentioned below.

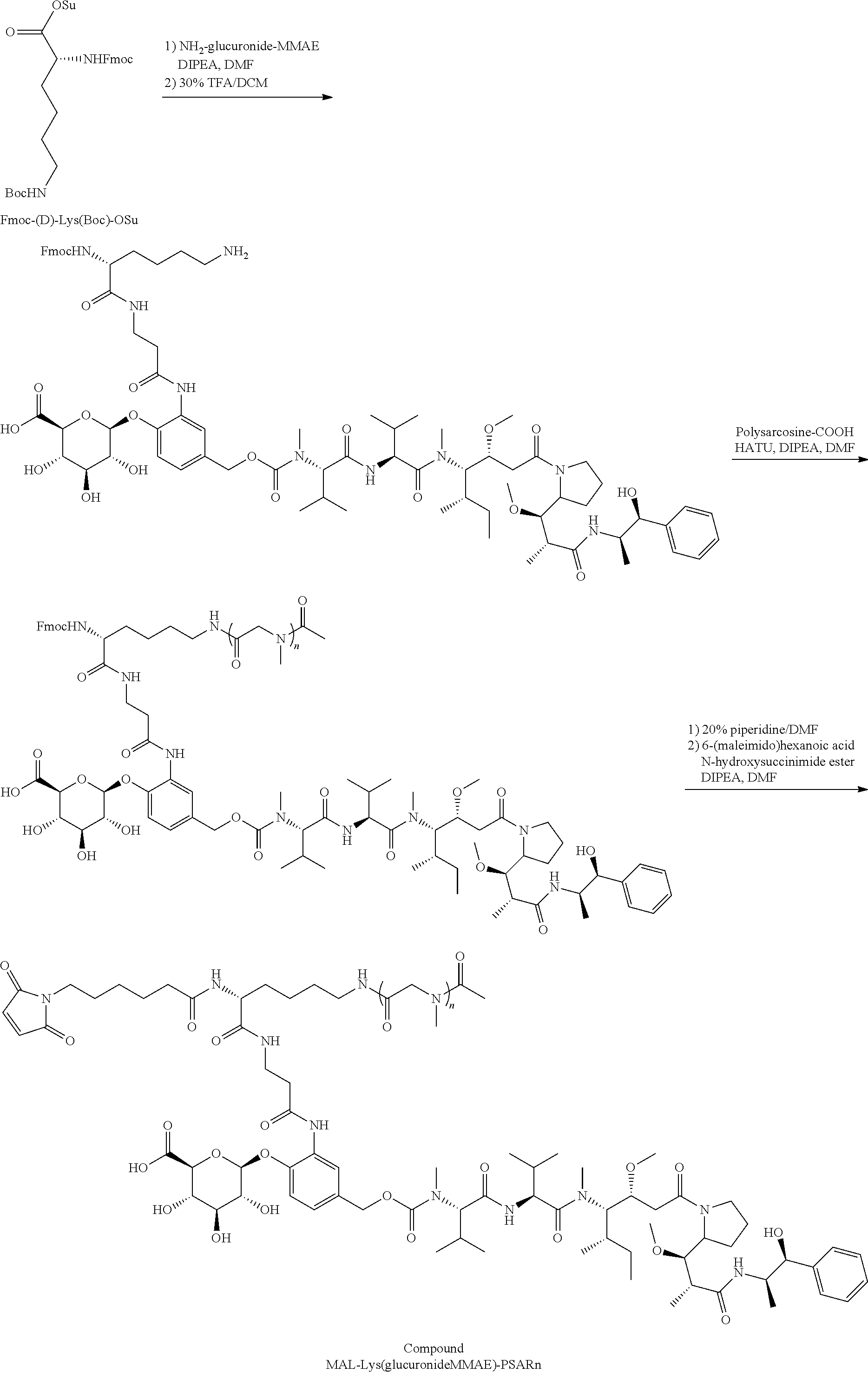

### 8.1) Synthesis of Fmoc-D-Lys(glucuronideMMAE)-NH₂

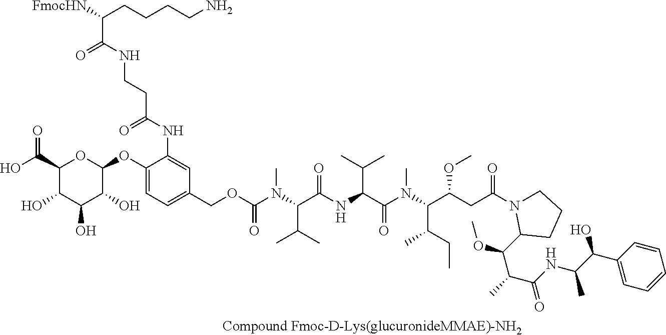

Compound (2R,3R,4R,5S,6R)-6-(2-(3-aminopropanamido)-4-((5S,8S,11S,12R)-11-((S)-sec-butyl)-12-(2-(2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (“NH₂-glucuronide-MMAE”; synthesized as described in Jeffrey SC et al., Bioconjug. Chem., 2006, 17(3), 831-840) (69.9 mg/0.062 mmol) and Fmoc-D-Lys(Boc)-OSu (35 mg/0.062 mmol) were dissolved in 1.2 mL of anhydrous DMF. DIPEA (24.0 mg/0.186 mmol) was added and the mixture was stirred 20 hours at room temperature. Volatiles were removed under vacuum. The flask containing the slightly yellow crude was put on an ice bath (0° C.) and 7 mL of a DCM/TFA (7:3 v/v) solution was slowly added. The solution was stirred on ice until entire Boc deprotection was observed by HPLC (approximately 2 hours). Volatiles were then removed under vacuum and the residue was taken up in DMF for purification on a 30 g Biotage® SNAP Ultra C18 (25 μm) cartridge. Mobile phase A was water+0.05% TFA and mobile phase B was acetonitrile+0.05% TFA. The gradient ranged from 10 to 60% B. Compound Fmoc-D-Lys(glucuronideMMAE)-NH₂ was obtained as a white solid (59 mg/65%). LC-HRMS m/z (ESI⁺): Calc [M+H]⁺=1480.7862; Exp [M+H]⁺=1480.7890; Error=−1.9 ppm. HPLC Method 1 retention time=10.5 min.

### 8.2) Synthesis of Fmoc-D-Lys(glucuronideMMAE)-PSAₙ

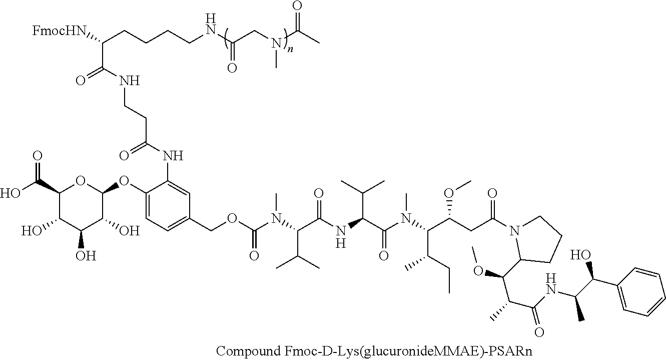

Compound PSARn-COOH (2 eq; obtained as described in Example 2 and previously dissolved as a 0.15M stock solution in anhydrous DMF) was added onto HATU (1.8 eq) in a vial. DIPEA (5 eq) was added and the mixture was stirred 3 minutes at room temperature. Compound Fmoc-D-Lys(glucuronideMMAE)-NH₂ (1 eq; previously dissolved as a 0.05M stock solution in anhydrous DMF) was then added. The mixture was stirred for 1 hour at room temperature and was injected on a 30 g Biotage® SNAP Ultra C18 (25 μm) cartridge for purification. Mobile phase A was water+0.05% TFA and mobile phase B was acetonitrile+0.05% TFA. The gradient ranged from 10 to 60% B.

Compound Fmoc-D-Lys(glucuronideMMAE)-PSAR6 was obtained as a white solid (9.8 mg/38%). LC-HRMS m/z (ESI): Calc [M+2H]²⁺=975.0133; Exp [M+2H]²⁺=975.0088; Error=4.6 ppm. HPLC Method 1 retention time=7.5 min.

Compound Fmoc-D-Lys(glucuronideMMAE)-PSAR12 was obtained as a white solid (3.6 mg/28%). LC-HRMS m/z (ESI): Calc [M+2H]²+=1188.1247; Exp [M+2H]²⁺=1188.1233; Error=1.1 ppm. HPLC Method 1 retention time=7.6 min.

### 8.3) 6-(Maleimido)Hexanoic Acid N-Hydroxysuccinimide Ester Coupling Procedure

Compound Fmoc-D-Lys(glucuronideMMAE)-PSARn from the previous step was treated with 20% piperidine in DMF at room temperature for 5 minutes. Volatiles were removed under high vacuum, and the dry residue was dissolved in anhydrous DMF. Then, 6-(maleimido)hexanoic acid N-hydroxysuccinimide ester (8 eq) and DIPEA (10 eq) were added and the mixture was agitated at room temperature for 30 min. The reaction mixture was quenched with water/TFA (99.5:0.5 v/v) and purified on a 30 g Biotage® SNAP Ultra C₁₈ (25 μm) cartridge. Mobile phase A was water+0.05% TFA and mobile phase B was acetonitrile+0.05% TFA. After a 10 min isocratic hold (5% B), compound of interest was eluted isocratically with 40% B.

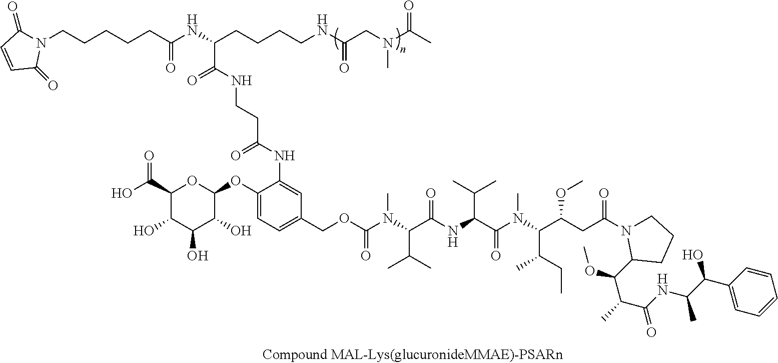

Compound MAL-Lys(glucuronideMMAE)-PSAR6 was obtained as a transparent oil (5.0 mg/52%). LC-HRMS m/z (ESI⁺): Calc [M+2H]²⁺=960.5163; Exp [M+2H]²⁺=960.5167; Error=−0.5 ppm. HPLC Method 1 retention time=7.1 min.

Compound MAL-Lys(glucuronideMMAE)-PSAR12 was obtained as a transparent oil (1.8 mg/51%). LC-HRMS m/z (ESI⁺): Calc [M+2H]²⁺=1173.6276; Exp [M+2H]²⁺1173.6229; Error=4.0 ppm. HPLC Method 1 retention time=7.0 min.

### Example 9: Synthesis of Polysarcosine-Based or Polyethyleneglycol-Based Drug Conjugate Linkers Using Glycine as Orthogonal Moiety

### 9.1) Compound bromoacetamide-Ngly(triazole- glucuronideMMAE)-PSARn

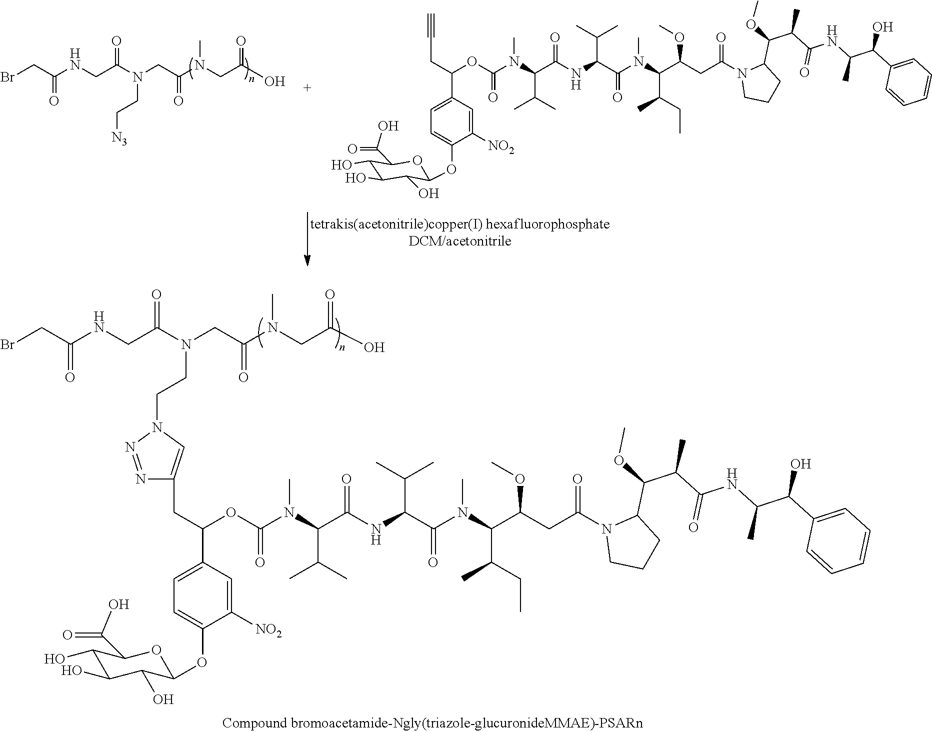

Alkyne-glucuronide-MMAE (1 eq; obtained as described in Example 6), PSARn-N₃-bromoacetamide (1.1 eq; obtained as described in Example 3) and tetrakis(acetonitrile)copper(I) hexafluorophosphate (3 eq) were combined in a reaction vessel. DCM/acetonitrile 1:1 (v/v) solution was added to reach a final alkyne-glucuronide-MMAE concentration of 12 μmol/mL. The reaction was stirred 16-20 hours at room temperature under argon in the dark. After removal of the volatiles under reduced pressure, the residue was taken up in DMF and purified on a 30 g Biotage® SNAP Ultra C18 (25 μm) cartridge. Mobile phase A was water+0.1% TFA and mobile phase B was acetonitrile+0.1% TFA. The gradient ranged from 10 to 50% B.

Compound bromoacetamide-Ngly(triazole-glucuronideMMAE)-PSAR12 was obtained as a white solid (8.5 mg/51%). LC-HRMS m/z (ESI⁺): Calc [M+2H]²⁺=1151.0179; Exp [M+2H]²⁺=1151.0188; Error=−0.8 ppm. HPLC Method 3 retention time=8.5 min.

### 9.2) Compound MAL-phenyl-Ngly(triazole-glucuronideMMAE)-PSARn

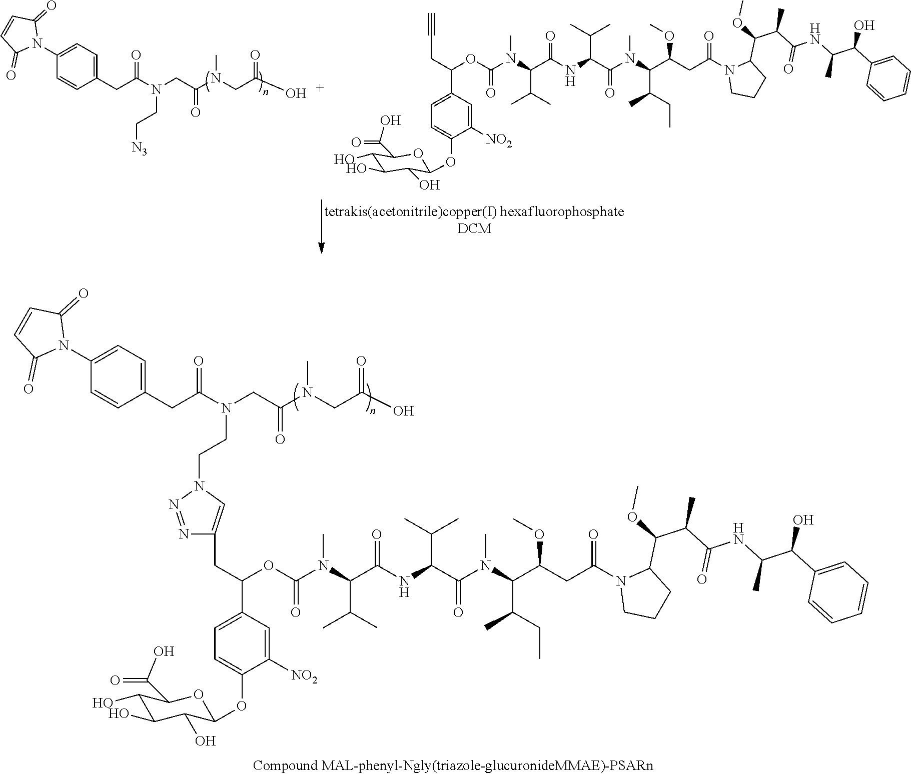

Alkyne-glucuronide-MMAE (obtained as described in Example 6) and PSARn-N₃-phenyl-MAL (obtained as described in Example 3) were reacted and purified as described above in section 9.1, using DCM as reaction solvent.

Compound MAL-phenyl-Ngly(triazole-glucuronideMMAE)-PSAR6 was obtained as a white solid (3.3 mg/20%). LC-HRMS m/z (ESI⁺): Calc [M+2H]²⁺=955.9566; Exp [M+2H]²⁺=955.9533; Error=3.4 ppm. HPLC Method 3 retention time=9.2 min.

Compound MAL-phenyl-Ngly(triazole-glucuronideMMAE)-PSAR12 was obtained as a white solid (9.0 mg/33%). LC-HRMS m/z (ESI⁺): Calc [M+2H]²⁺=1169.0679; Exp [M+2H]²⁺=1169.0621; Error=4.9 ppm. HPLC Method 3 retention time=8.7 min.

Compound MAL-phenyl-Ngly(triazole-glucuronideMMAE)-PSAR18 was obtained as a white solid (11.5 mg/40%). LC-HRMS m/z (ESI⁺): Calc [M+2H]²⁺=1382.1792; Exp [M+2H]²⁺=1382.1803; Error=−0.7 ppm. HPLC Method 3 retention time=8.6 min.

Compound MAL-phenyl-Ngly(triazole-glucuronideMMAE)-PSAR24 was obtained as a white solid (15 mg/44%). LC-HRMS m/z (ESI⁺) Calc [M+4Na]⁴⁺=820.1309; Exp [M+4Na]⁴⁺=820.1324; Error=−1.8 ppm. HPLC Method 3 retention time=8.4 min.

### 9.3) Compound MAL-phenyl-Ngly(triazole-glucuronideMMAE)-PEGn

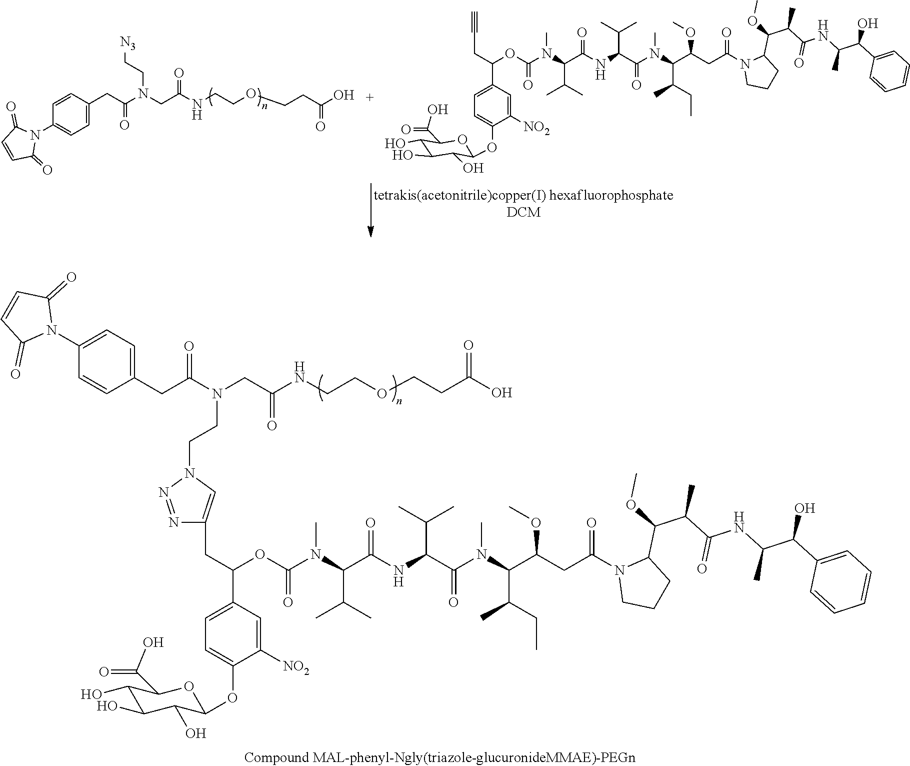

Alkyne-glucuronide-MMAE (obtained as described in Example 6) and PEGn-N₃-phenyl-MAL (obtained as described in Example 5) were reacted and purified as described above in section 9.1, using NMP/DCM 2:1 (v/v) as reaction solvent.

Compound MAL-phenyl-Ngly(triazole-glucuronideMMAE)-PEG12 was obtained as a slightly yellow oil (10.4 mg/45%). LC-HRMS m/z (ESI⁺): Calc [M+2H]²⁺=1042.5211; Exp [M+2H]²⁺=1042.5218; Error=−0.7 ppm. HPLC Method 3 retention time=8.0 min.

### 9.4) Compound MAL-phenyl-Ngly(triazole-glucuronideMMAE)-Ngly(triazole-glucuronideMMAE)-PSARn

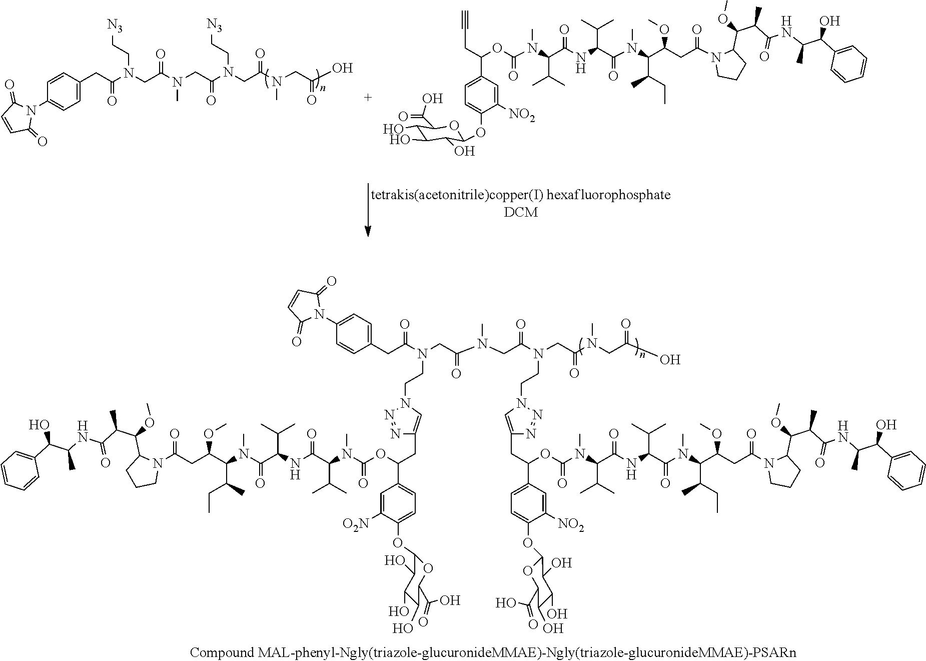

Alkyne-glucuronide-MMAE (3 eq; obtained as described in Example 6), PSARn-N₃—N₃-phenyl-MAL (1 eq; obtained as described in Example 3) and tetrakis(acetonitrile)copper(I) hexafluorophosphate (5 eq) were combined in a reaction vessel. DCM was added and the reaction was stirred 16-20 hours at room temperature under argon in the dark. After removal of the volatiles under reduced pressure, the residue was taken up in DMF and purified on a 30 g Biotage® SNAP Ultra C18 (25 μm) cartridge. Mobile phase A was water+0.1% TFA and mobile phase B was acetonitrile +0.1% TFA. The gradient ranged from 10 to 50% B.

Compound MAL-phenyl-Ngly(triazole-glucuronideMMAE)-Ngly(triazole-glucuronideMMAE)-PSAR18 was obtained as a white solid (10.1 mg/44%). LC-HRMS m/z (ESIJ) Calc [M+4H]⁴⁺=1022.5082; Exp [M+4H]⁴⁺=1022.5093; Error=−1.0 ppm. HPLC Method 3 retention time=9.5 min.

### 9.5) Compound MAL-phenyl-Ngly[triazole-glucuronide(MMAE)₂]-PSARn

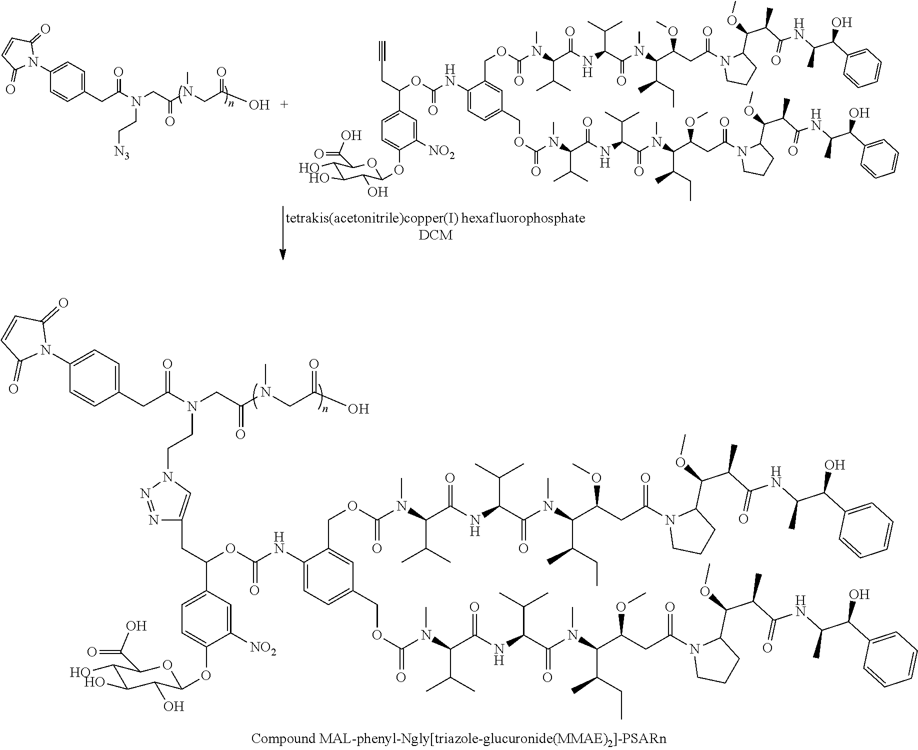

Alkyne-glucuronide-(MMAE)₂ (obtained as described in Example 6) and PSARn-N₃-phenyl-MAL (obtained as described in Example 3) were reacted and purified as described above in section 9.1, using DCM as reaction solvent.

Compound MAL-phenyl-Ngly[triazole-glucuronide(MMAE)₂]-PSAR24 was obtained as a white solid (12.5 mg/39%). LC-HRMS m/z (ESI⁺): Calc [M+3H]³⁺=1371.3767; Exp [M+3H]³⁺=1371.3818; Error=−3.8 ppm. HPLC Method 3 retention time=10.0 min.

### 9.6) Compound MAL-phenyl-Ngly[triazole-galactoside(MMAE)₂]-PSARn

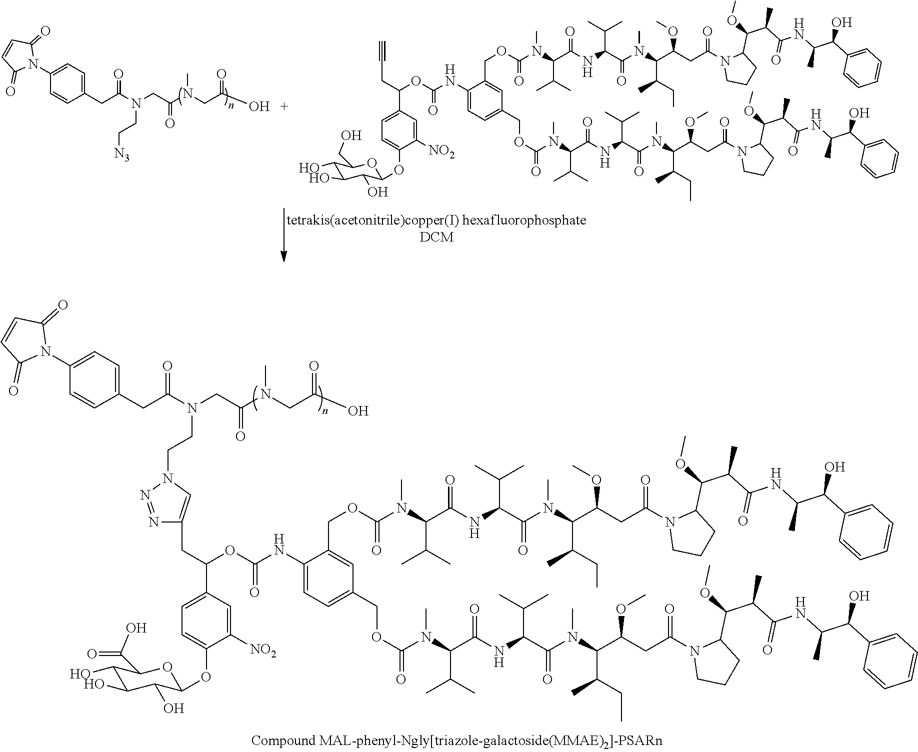

Alkyne-galactoside-(MMAE)₂ (synthesized as described in Alsarraf et al., Chem. Commun., 2015, 51(87), 15792-15795) and PSARn-N₃-phenyl-MAL (obtained as described in Example 3) were reacted and purified as described above in section 9.1, using DCM as reaction solvent.

Compound MAL-phenyl-Ngly[triazole-galactoside(MMAE)₂]-PSAR24 was obtained as a white solid (6.5 mg/55%). LC-HRMS m/z (ESI⁺): Calc [M+4H]⁴⁺=1022.7895; Exp [M+4H]⁴⁺=1022.7903; Error=0.8 ppm. HPLC Method 3 retention time=9.2 min.

### 9.7) Compound MAL-phenyl-Ngly(triazole-val-cit-PAB-MMAE)-PSARn

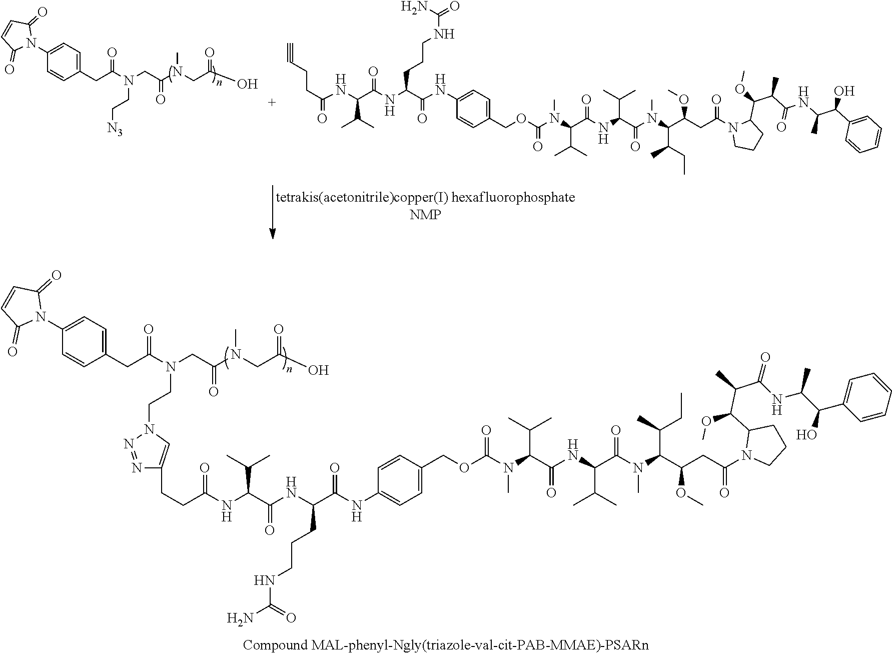

Alkyne-val-cit-PAB-MMAE (obtained as described in Example 6) and PSARn-N₃-phenyl-MAL (obtained as described in Example 3) were reacted and purified as described above in section 9.1, using NMP as reaction solvent.

Compound MAL-phenyl-Ngly(triazole-val-cit-PAB-MMAE)-PSAR12 was obtained as a white solid (4.5 mg/12%). LC-HRMS m/z (ESI⁺): Calc [M+2H]²⁺=1207.1494; Exp [M+2H]²⁺=1207.1535; Error=−3.5 ppm. HPLC Method 3 retention time=8.6 min.

### 9.8) Compound MAL-phenyl-Ngly(triazole-SN38)-PSARn

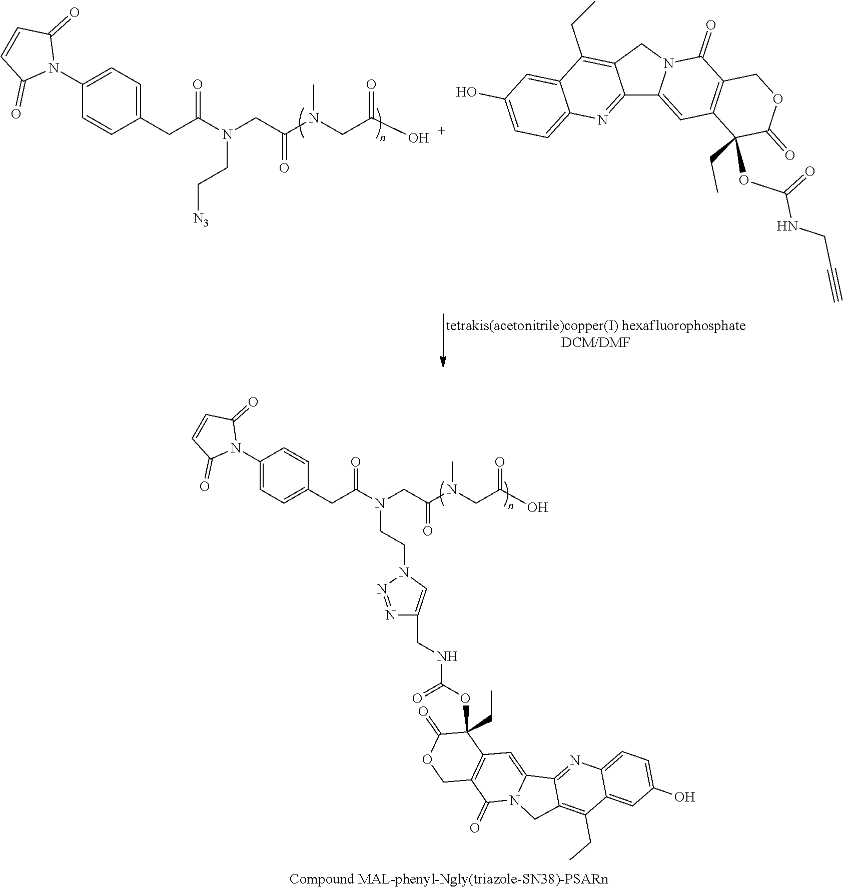

Alkyne-SN38 (obtained as described in Example 6) and PSARn-N₃-phenyl-MAL (obtained as described in Example 3) were reacted and purified as described above in section 9.1, using DCM/DMF 2:1 (v/v) as reaction solvent.

Compound MAL-phenyl-Ngly(triazole-SN38)-PSAR18 was obtained as a bright yellow solid (4.0 mg/20%). LC-HRMS m/z (ESI⁺): Calc [M+2Na]²⁺=1077.4562; Exp [M+2Na]²⁺=1077.4588; Error=−2.4 ppm. HPLC Method 3 retention time=7.5 min.

### 9.9) Compound MAL-phenyl-Ngly(triazole-SN38)-Ngly(triazole-SN38)-PSARn

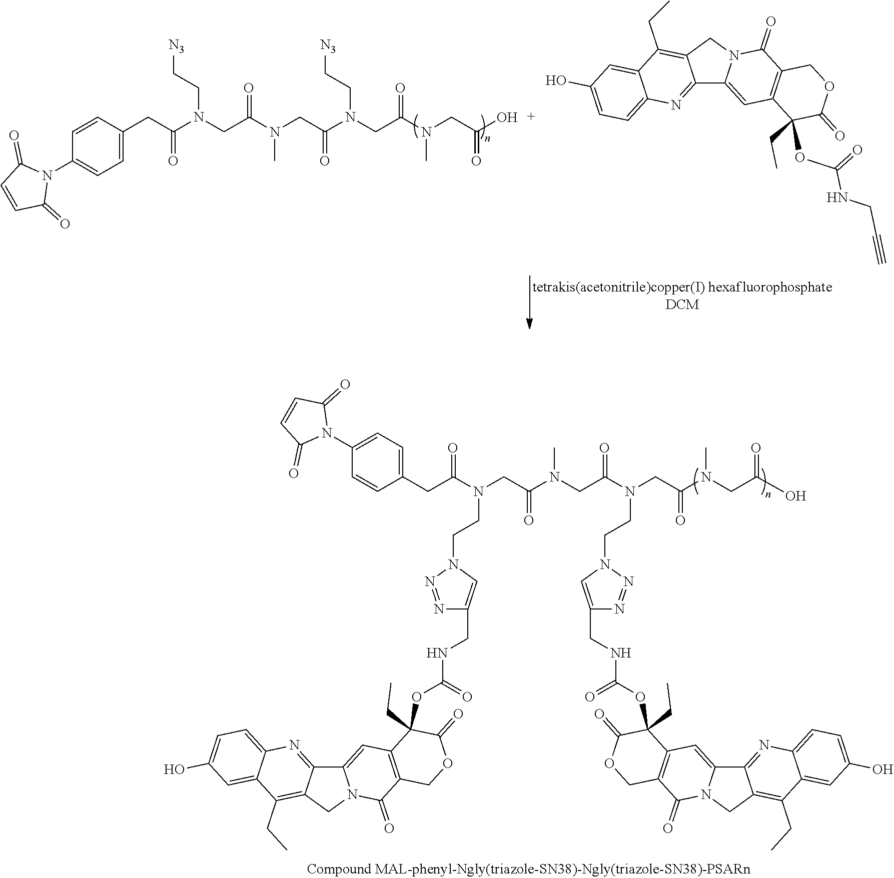

Alkyne-SN38 (obtained as described in Example 6) and PSARn-N₃—N₃-phenyl-MAL (obtained as described in Example 3) were reacted and purified as described above in section 9.4.

Compound MAL-phenyl-Ngly(triazole-SN38)-Ngly(triazole-SN38)-PSAR18 was obtained as a yellow solid (5.1 mg/43%). LC-HRMS m/z (ESI⁺): Calc [M+2Na]²⁺=1412.5812; Exp [M+2Na]²⁺=1412.5852; Error=−3.8 ppm. HPLC Method 3 retention time=8.4 min.

### 9.10) Compound MAL-phenyl-Ngly(triazole-glucuronideSN38)-Ngly(triazole-glucuronideSN38)-PSARn

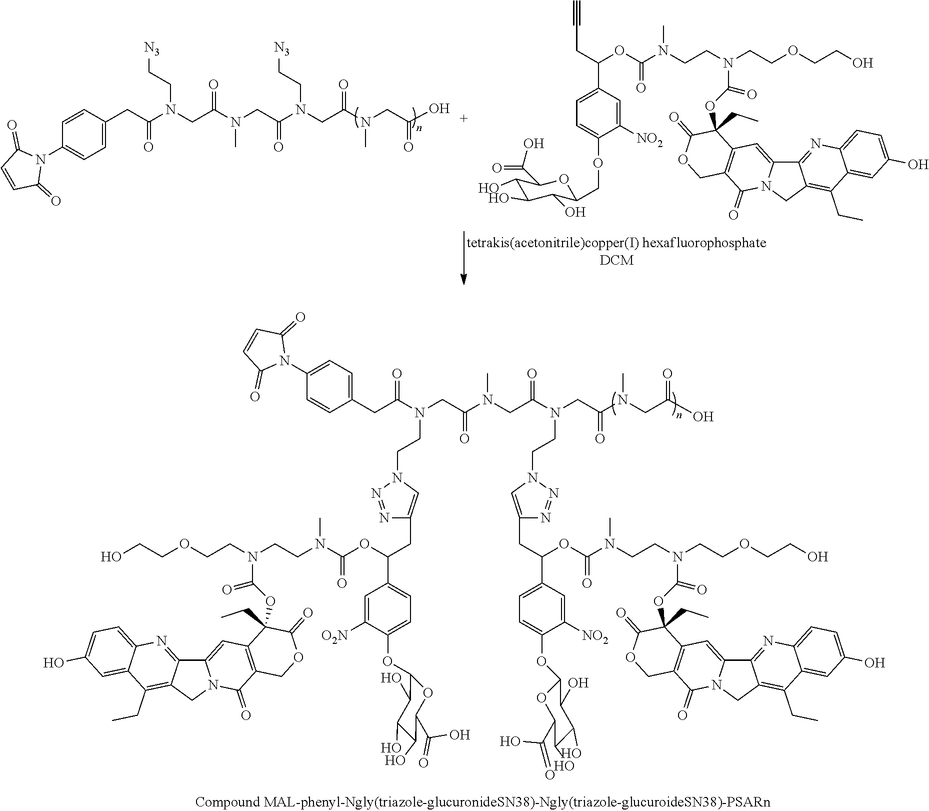

Alkyne-glucuronide-SN38 (obtained as described in Example 6) and PSARn-N₃—N₃-phenyl-MAL (obtained as described in Example 3) were reacted and purified as described above in section 9.4, using DCM/MeOH 8:2 (v/v) as reaction solvent.

Compound MAL-phenyl-Ngly(triazole-glucuronideSN38)-Ngly(triazole-glucuronideSN38)-PSAR18 was obtained as a yellow solid (7.0 mg/30%). LC-HRMS m/z (ESI⁺): Calc [M+2H]²⁺=1271.5080; Exp [M+2H]²⁺=1271.5103; Error=−1.8 ppm. HPLC Method 3 retention time=7.8 min.

### 9.11) Compound MAL-phenyl-Ngly(triazole-glucuronide-Exatecan)-PSARn

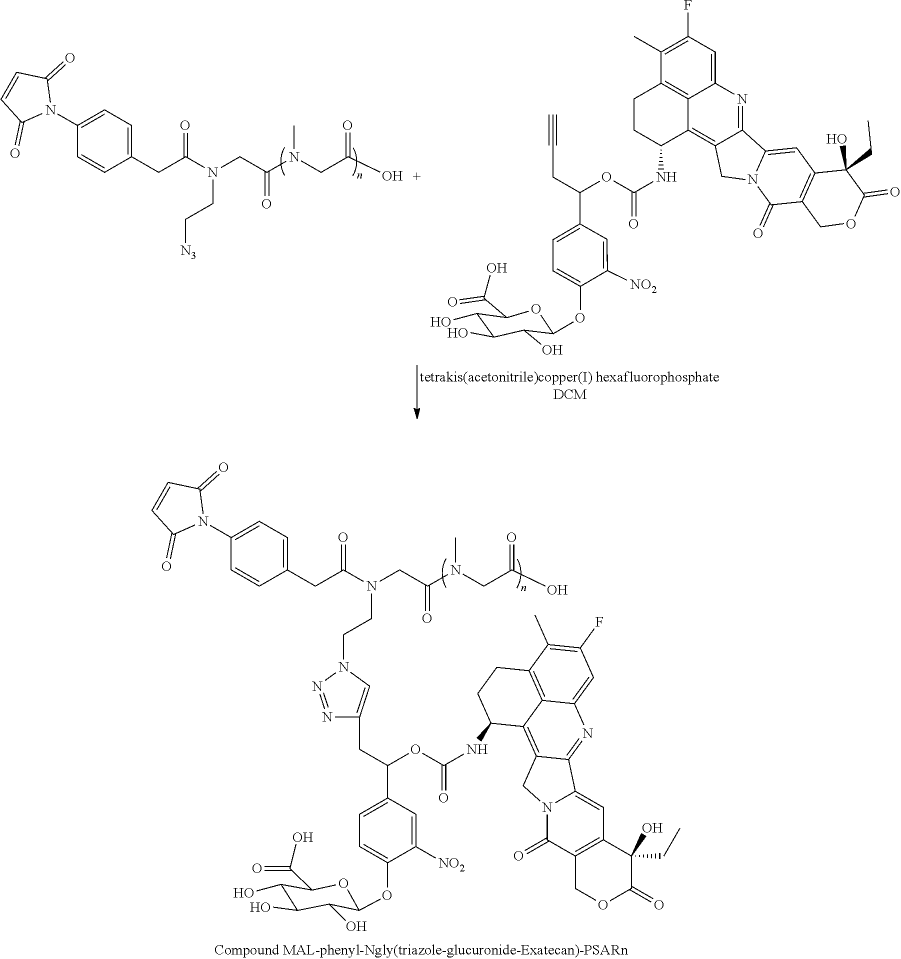

Alkyne-glucuronide-Exatecan (obtained as described in Example 6) and PSARn-N₃-phenyl-MAL (obtained as described in Example 3) were reacted and purified as described above in section 9.1, using DCM as reaction solvent.

Compound MAL-phenyl-Ngly(triazole-glucuronide-Exatecan)-PSAR18 was obtained as a yellow solid (13.2 mg/64%). LC-HRMS m/z (ESI⁺). Calc [M+2H]²⁺=1241.0069; Exp [M+2H]²⁺=1241.0088; Error=−1.1 ppm. HPLC Method 3 retention time=7.1 min.

### 9.12) Compound MAL-phenyl-Ngly(triazole-PNU159682)-PSARn

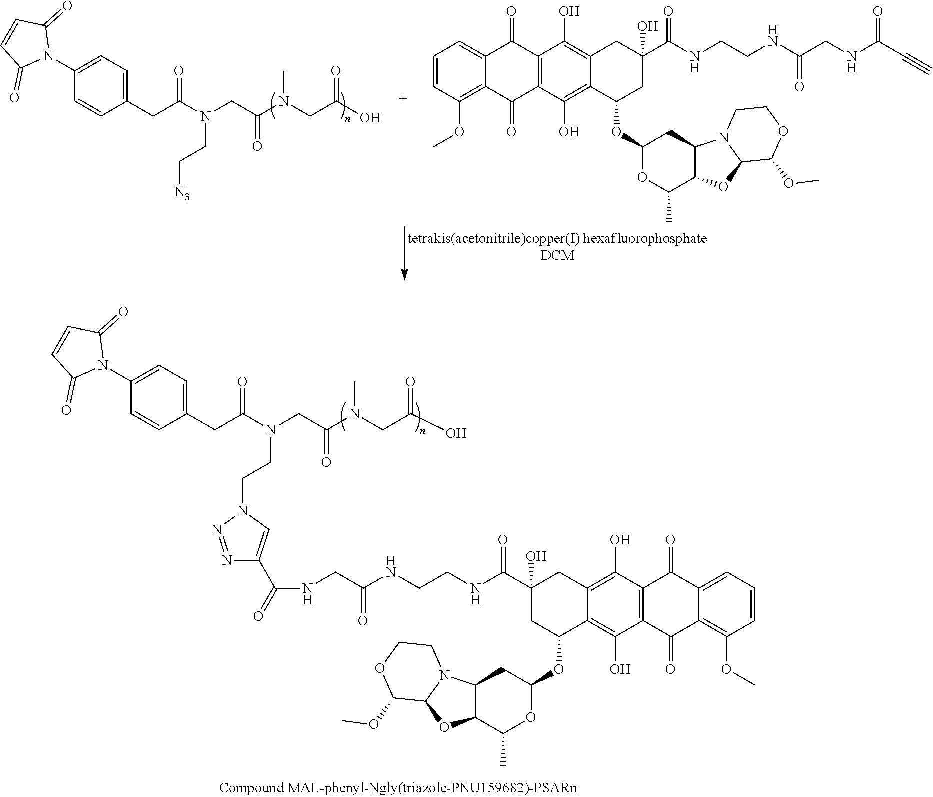

Alkyne-PNU159682 (obtained as described in Example 6) and PSARn-N₃-phenyl-MAL (obtained as described in Example 3) were reacted and purified as described above in section 9.1, using DCM as reaction solvent and replacing 0.1% TFA additive in mobile phases during reverse phase purification with 0.1% formic acid. Compound MAL-phenyl-Ngly(triazole-PNU159682)-PSAR12 was obtained as a red solid (4.8 mg/40%). LC-HRMS m/z (ESI⁺): Calc [M+2H]²⁺=994.9185; Exp [M+2H]²⁺=994.9184; Error=0.1 ppm. HPLC Method 6 retention time=5.0 min. Compound MAL-phenyl-Ngly(triazole-PNU159682)-PSAR18 was obtained as a red solid (7.2 mg/33%). LC-HRMS m/z (ESI⁺): Calc [M+2H]²⁺=1208.0298; Exp [M+2H]²⁺=1208.0295; Error=0.3 ppm. HPLC Method 6 retention time=4.9 min.

### Example 10: Synthesis of negative control drug conjugate linkers MAL-glucuronideMMAE, MAL-phenyl-triazole-glucuronideMMAE and MAL-phenyl-PSARn-triazole-glucuronideMMAE

### 10.1) Synthesis of compound MAL-glucuronideMMAE

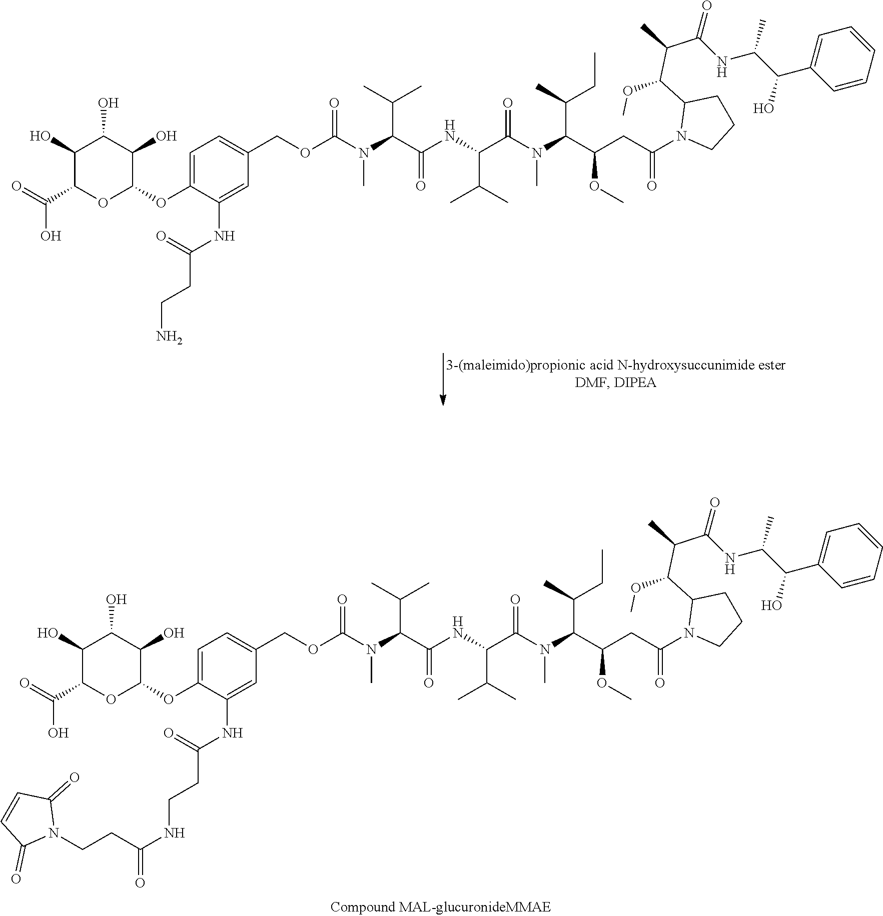 [SMILES: CC[C@H](C)[C@@H]([C@@H](CC(=O)N1CCCC1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCN)c1)C(C)C ; CC[C@H](C)[C@@H]([C@@H](CC(=O)N1CCCC1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1)C(C)C]

Starting compound (2R,3R,4R,5S,6R)-6-(2-(3-aminopropanamido)-4-((5S,8S,11S,12R)-11-((S)-sec-butyl)-12-(2-(2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (“NH₂-glucuronide-MMAE”; synthesized as described in Jeffrey SC et al., Bioconjug. Chem., 2006, 17(3), 831-840) (6.2 mg/5 μmol) and 3-(maleimido)propionic acid N-hydroxysuccinimide ester (14.6 mg/55 μmol) were weighted and dissolved in 200 μL of anhydrous DMF. DIPEA (8.5 mg/66 μmol) was added and the mixture was agitated at room temperature for 30 min. The reaction mixture was quenched with 1.5 mL of water/TFA (99:1 v/v) and purified on a 30 g Biotage® SNAP Ultra C18 (25 μm) cartridge. Mobile phase A was water+0.05% TFA and mobile phase B was acetonitrile+0.05% TFA. The gradient ranged from 10 to 70% B.

Title compound MAL-glucuronideMMAE was obtained as a white solid (4.1 mg/59%). LC-HRMS m/z (ESI⁺): Calc [M+H]⁺=1281.6501; Exp [M+H]⁺=1281.6489; Error=0.9 ppm. HPLC Method 1 retention time=7.1 min.

### 10.2) Synthesis of compound MAL-phenyl-triazole-glucuronideMMAE

### 10.2.1) Synthesis of perfluorophenyl 2-(4-(2,5-dioxo-2,5-dihydro-1H-pyrrol-1-yl)phenyl)acetate

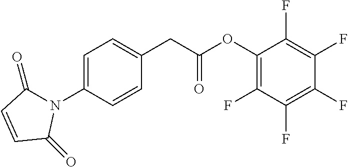 [SMILES: O=C(Cc1ccc(N2C(=O)C=CC2=O)cc1)Oc1c(F)c(F)c(F)c(F)c1F]

Commercially available 2-[4-(2,5-dioxo-2,5-dihydro-1H-pyrrol-1-yl)phenyl]acetic acid (299 mg/1.29 mmol), N,N′-Dicyclohexylcarbodiimide (267 mg/1.29 mmol) and pentafluorophenol (238 mg/1.29 mmol) were dissolved in 15 mL of anhydrous 1,2-Dimethoxyethane in a reaction vessel. After 2 hours of stirring at room temperature, insolubles were removed by filtration and the filtrate was purified by chromatography on silica gel (petroleum ether/EtOAc, gradient from 80:20 to 20:80) to afford title compound (400 mg/78%) as a white solid. ¹H NMR (300 MHz, CDCl₃) δ (ppm) 4.01 (s, 2H), 6.87 (s, 2H), 7.40 (d, J=8.7 Hz, 2H), 7.47 (d, J=8.7 Hz, 2H). HRMS m/z (ESI⁺): Calc [M+H]⁺=398.0446; Exp [M+H]⁺=398.0448; Error=−0.4 ppm.

### 10.2.2) Synthesis of N-(2-azidoethyl)-2-(4-(2,5-dioxo-2,5-dihydro-1H-pyrrol-1-yl)phenyl)acetamide

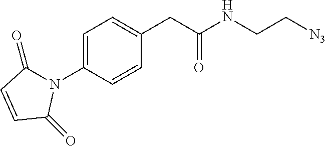 [SMILES: [N-]=[N+]=NCCNC(=O)Cc1ccc(N2C(=O)C=CC2=O)cc1]

Previous compound perfluorophenyl 2-(4-(2,5-dioxo-2,5-dihydro-1H-pyrrol-1-yl)phenyl)acetate (78 mg/0.20 mmol) was dissolved in 1 mL of anhydrous DCM in a reaction vessel. 2-azidoethan-1-amine (33.8 mg/0.40 mmol) was added and the reaction was stirred 1 hour at room temperature. 1N HCl solution was then added and the mixture was extracted 3 times with DCM. The organic phase was dried over MgSO₄, filtered and evaporated under vacuum to afford a solid crude that was purified by chromatography on silica gel (petroleum ether/EtOAc, gradient from 60:40 to 0:100) to afford title compound (18 mg/31%) as a white solid. MS (ESI⁺): [M+H]⁺=300.1; HPLC Method 1 retention time=8.4 min. TLC eluting with 100% EtOAc: Rf=0.65.

### 10.2.3) Synthesis of compound MAL-phenyl-triazole-glucuronideMMAE

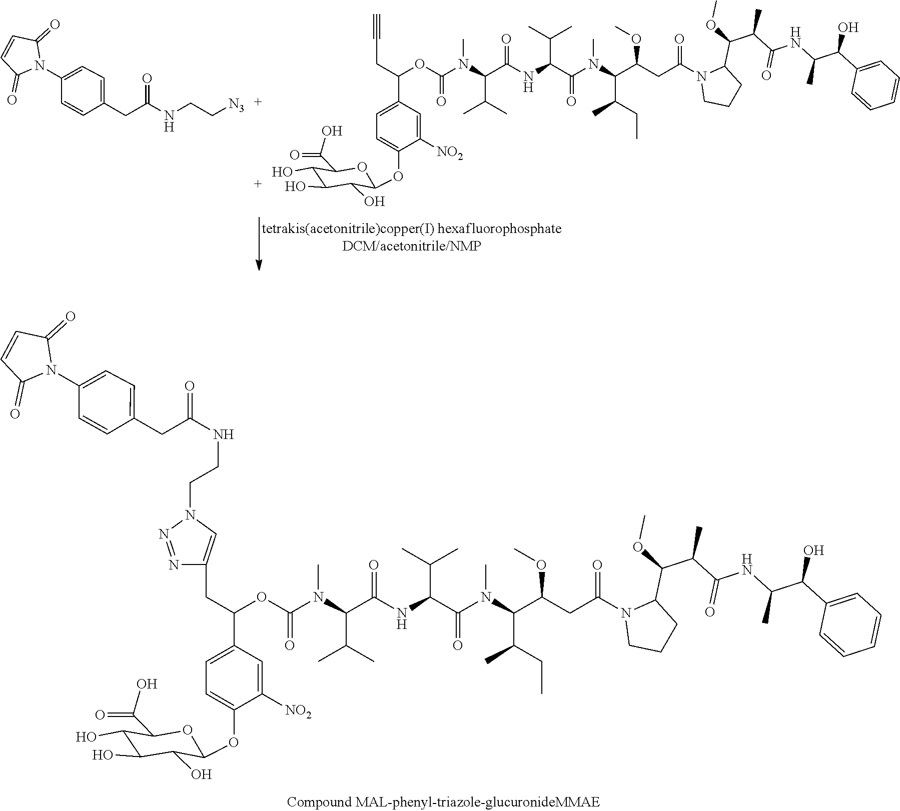 [SMILES: [N-]=[N+]=NCCNC(=O)Cc1ccc(N2C(=O)C=CC2=O)cc1 ; C#CCC(OC(=O)N(C)[C@@H](C(=O)N[C@H](C(=O)N(C)[C@H]([C@H](C)CC)[C@H](CC(=O)N1CCCC1[C@@H](OC)[C@@H](C)C(=O)N[C@H](C)[C@@H](O)c1ccccc1)OC)C(C)C)C(C)C)c1ccc(OC2OC(C(=O)O)C(O)C(O)C2O)c([N+](=O)[O-])c1 ; CC[C@@H](C)[C@H]([C@H](CC(=O)N1CCCC1[C@@H](OC)[C@@H](C)C(=O)N[C@H](C)[C@@H](O)c1ccccc1)OC)N(C)C(=O)[C@@H](NC(=O)[C@@H](C(C)C)N(C)C(=O)OC(Cc1cn(CCNC(=O)Cc2ccc(N3C(=O)C=CC3=O)cc2)nn1)c1ccc(OC2OC(C(=O)O)C(O)C(O)C2O)c([N+](=O)[O-])c1)C(C)C]

Compound alkyne-glucuronideMMAE from Example 6 (17 mg/15.1 μmol), tetrakis(acetonitrile)copper(I) hexafluorophosphate (11.2 mg/30 μmol) and N-(2-azidoethyl)-2-(4-(2,5-dioxo-2,5-dihydro-1H-pyrrol-1-yl)phenyl)acetamide from previous step (6.3 mg/21 μmol) were combined in a HPLC vial. 800 μL of an anhydrous DCM/acetonitrile/NMP 1:1:1 (v/v/v) solution was added and the reaction was stirred 16 hours at room temperature under argon. After removal of the volatiles under reduced pressure, the residue was taken up in DMF and purified on a 30 g Biotage® SNAP Ultra C18 (25 μm) cartridge. Mobile phase A was water+0.1% TFA and mobile phase B was acetonitrile+0.1% TFA. The gradient ranged from 10 to 60% B.

Title compound MAL-phenyl-triazole-glucuronideMMAE was obtained as an off-white solid (10.3 mg/48%). LC-HRMS m/z (ESI⁺): Calc [M+2H]²⁺=713.8425; Exp [M+2H]²⁺=713.8415; Error=1.3 ppm. HPLC Method 3 retention time=10.2 min.

### 10.3) Synthesis of compound MAL-phenyl-PSARn-triazole-glucuronideMMAE

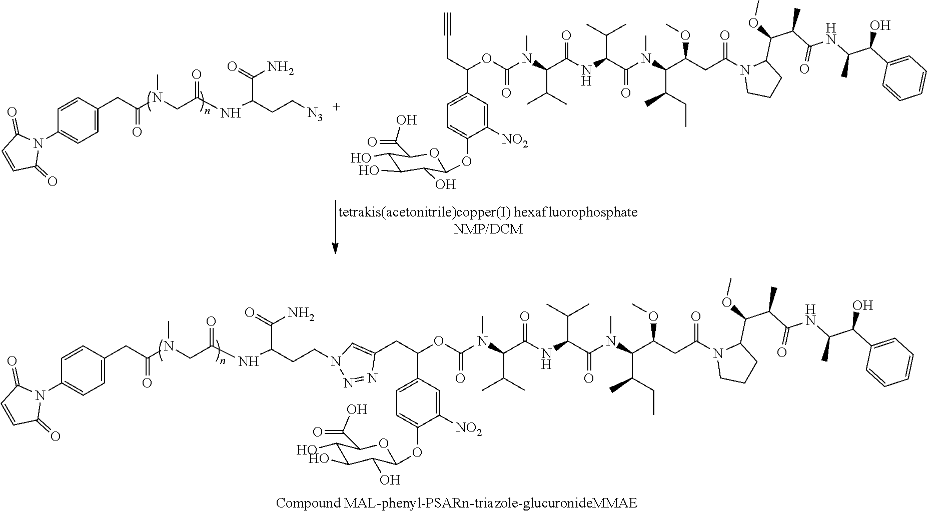

Compound alkyne-glucuronideMMAE from Example 6 (20 mg/17.7 μmol), tetrakis(acetonitrile)copper(I) hexafluorophosphate (13.2 mg/35 μmol) and N₃-PSARn-phenyl-MAL from Example 4 (34.3 mg/28 μmol) were combined in a HPLC vial. 900 μL of an NMP/DCM 2:1 (v/v) solution was added and the reaction was stirred 16 hours at room temperature under argon. After removal of the volatiles under reduced pressure, the residue was taken up in DMF and purified on a 30 g Biotage® SNAP Ultra C18 (25 μm) cartridge. Mobile phase A was water+0.10% TFA and mobile phase B was acetonitrile+0.1% TFA. The gradient ranged from 10 to 60% B.

Title compound MAL-phenyl-PSARn-triazole-glucuronideMMAE was obtained as a white solid (16.0 mg/39%). LC-HRMS m/z (ESI⁺): Calc [M+2H]²⁺=1168.5759; Exp [M+2H]²⁺=1168.5792; Error=−2.8 ppm. HPLC Method 3 retention time=6.8 min.

### Example 11: Preparation of LDC compounds of the invention

The following LDC compounds were prepared and characterized:
Trastuzumab-Glu(glucuronideMMAE)-CH₂—CH₂-PSAR6
Trastuzumab-Glu(glucuronideMMAE)-CH₂—CH₂-PSAR12
Trastuzumab-Glu(glucuronideMMAE)-CH₂—CH₂-PSAR18
Trastuzumab-Lys(glucuronideMMAE)-PSAR6
Trastuzumab-Lys(glucuronideMMAE)-PSAR12
Trastuzumab-BAC-Ngly(triazole-glucuronideMMAE)-PSAR12
Trastuzumab-MAL-phenyl-Ngly(triazole-glucuronideMMAE)-PSAR6
Trastuzumab-MAL-phenyl-Ngly(triazole-glucuronideMMAE)-PSAR12
Trastuzumab-MAL-phenyl-Ngly(triazole-val-cit-PAB-MMAE)-PSAR12
Trastuzumab-MAL-phenyl-Ngly(triazole-glucuronideMMAE)-PSAR18
Trastuzumab-MAL-phenyl-Ngly(triazole-SN38)-PSAR18
Trastuzumab-MAL-phenyl-Ngly(triazole-glucuronide-Exatecan)-PSAR18
Trastuzumab-MAL-phenyl-Ngly(triazole-PNU159682)-PSAR12
Trastuzumab-MAL-phenyl-Ngly(triazole-PNU159682)-PSAR18
Trastuzumab-MAL-phenyl-Ngly(triazole-glucuronideMMAE)-PSAR24
CD19-MAL-phenyl-Ngly(triazole-glucuronideMMAE)-PSAR24
CD22-MAL-phenyl-Ngly(triazole-glucuronideMMAE)-PSAR24
Trastuzumab-MAL-phenyl-Ngly(triazole-glucuronideMMAE)-Ngly(triazole-glucuronideMMAE)-PSAR18
Trastuzumab-MAL-phenyl-Ngly[triazole-glucuronide(MMAE)₂]-PSAR24
Trastuzumab-MAL-phenyl-Ngly[triazole-galactoside(MMAE)₂]-PSAR24
Trastuzumab-MAL-phenyl-Ngly(triazole-SN38)-Ngly(triazole-SN38)-PSAR18
Trastuzumab-MAL-phenyl-Ngly(triazole-glucuronideSN38)-Ngly(triazole-glucuronideSN38)-PSAR18
Trastuzumab-MAL-phenyl-Ngly(triazole-glucuronideMMAE)-PEG12
Trastuzumab-glucuronideMMAE
Trastuzumab-MAL-phenyl-triazole-glucuronideMMAE
Trastuzumab-MAL-phenyl-PSAR12-triazole-glucuronideMMAE
Human albumin-MAL-phenyl-Ngly(triazole-glucuronideMMAE)-PSAR24

Their structures are described in the following table 6.

TABLE 6

| Compound Name | Structure |
|---|---|
| Trastuzumab- Glu(glucuronide MMAE)-CH₂—CH₂- PSAR6 | 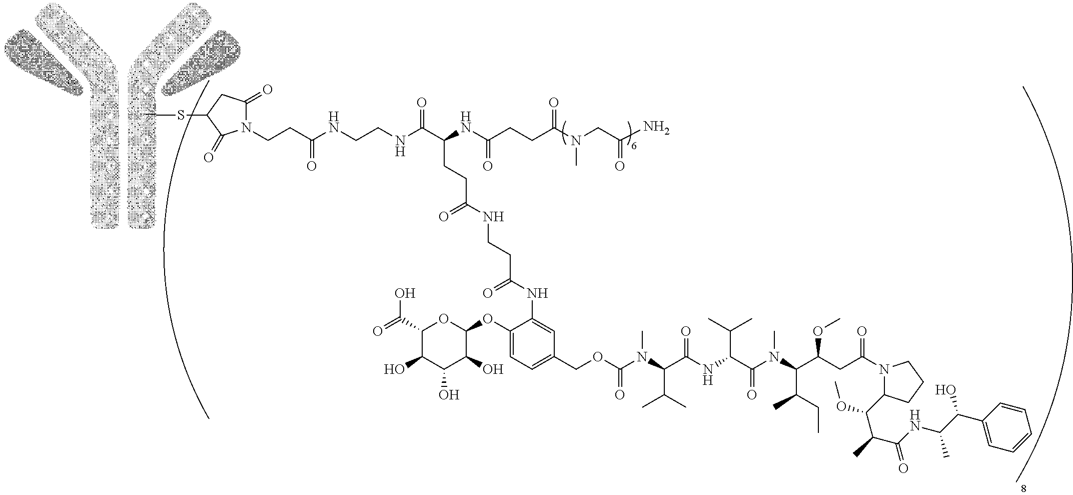 |
| Trastuzumab- Glu(glucuronide MMAE)-CH₂—CH₂- PSAR12 | 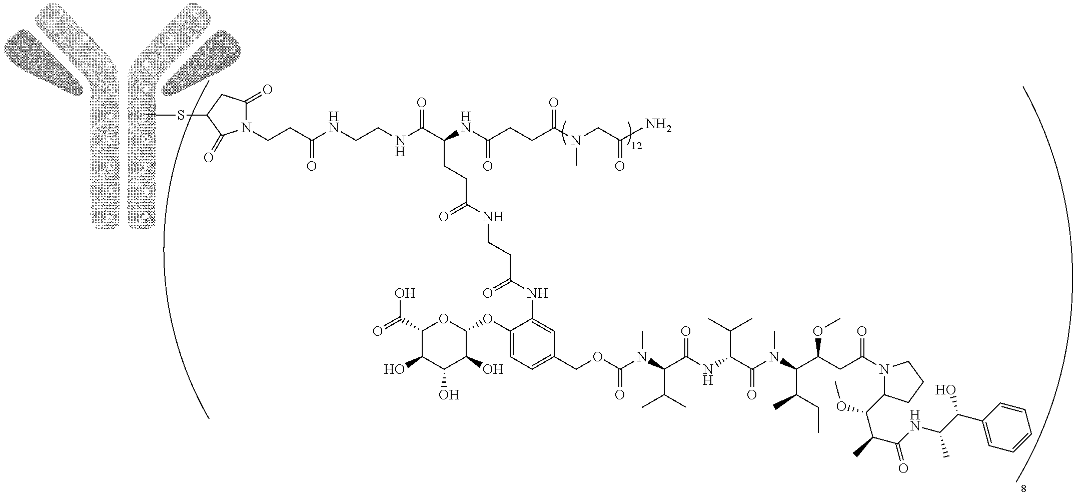 |
| Trastuzumab- Glu(glucuronide MMAE)-CH₂—CH₂- PSAR18 | 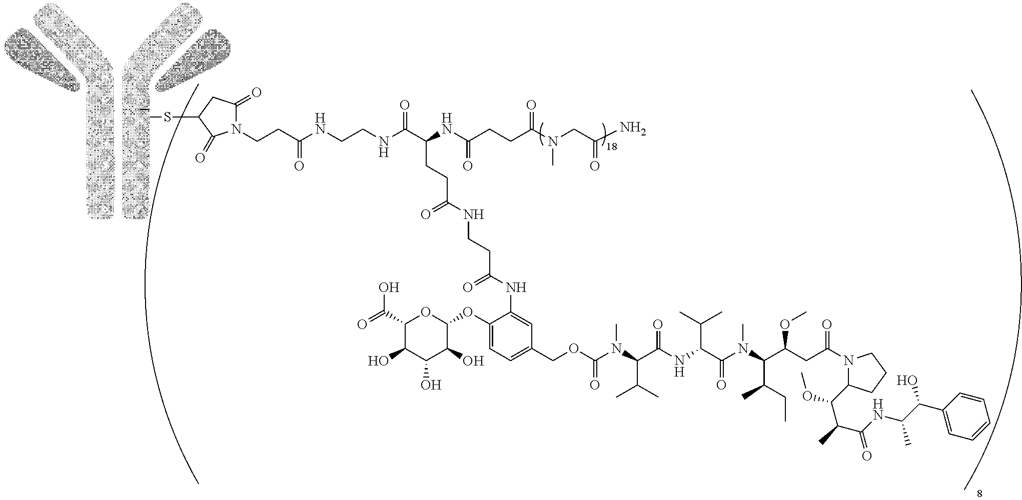 |
| Trastuzumab- Lys(glucuronide MMAE)-PSAR6 | 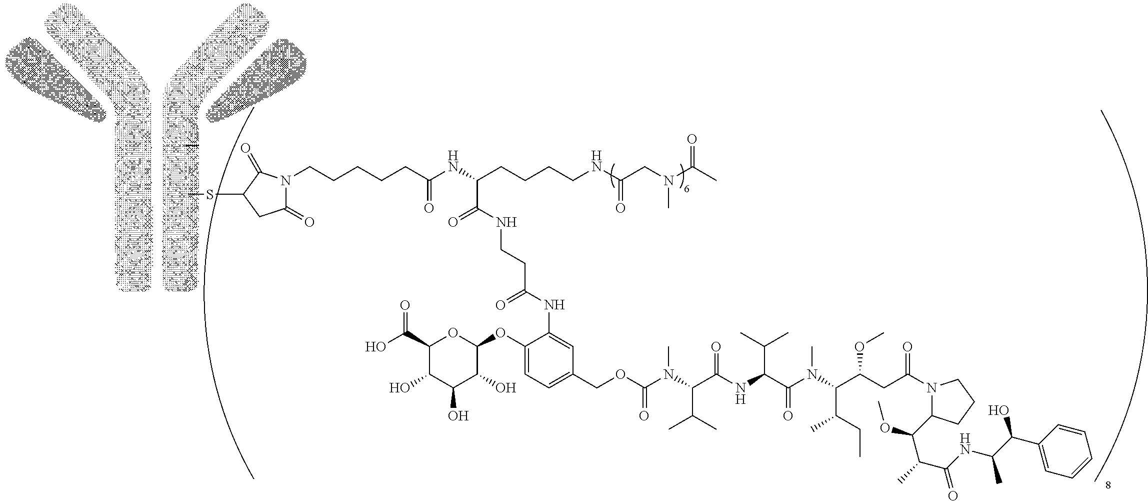 |
| Trastuzumab- Lys(glucoronide MMAE)-PSAR12 | 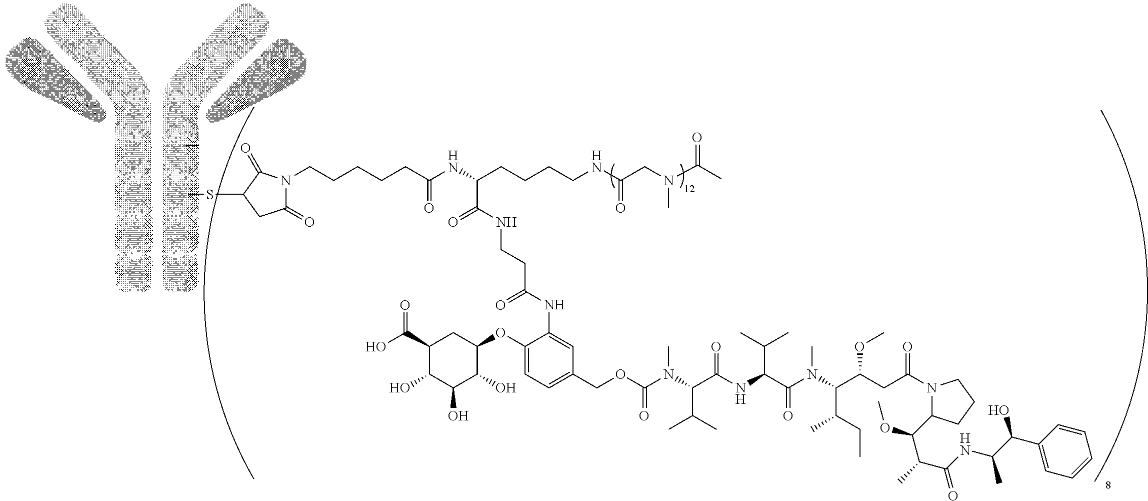 |
| Trastuzumab-BAC- Ngly(triazole- glucuronideMMAE)- PSAR12 | 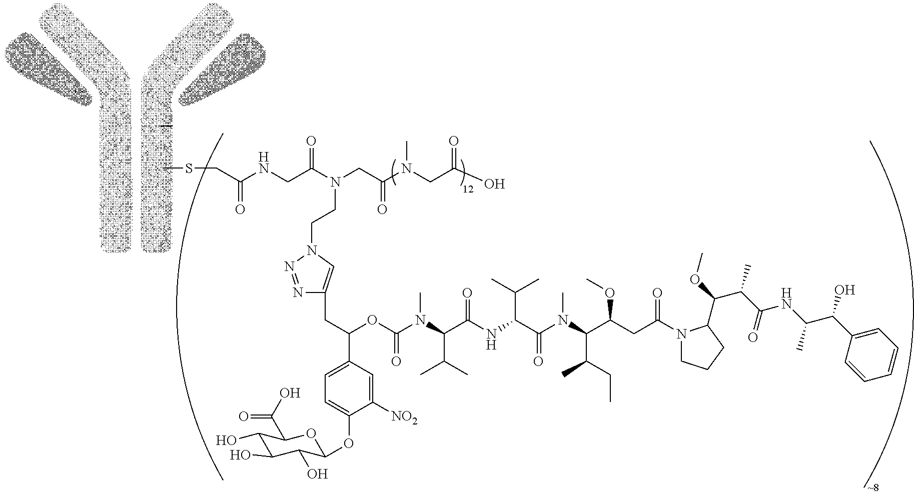 |
| Trastuzumab-MAL- phenyl-Ngly(triazole- glucuronideMMAE)- PSAR6 (ADC-PSAR6) | 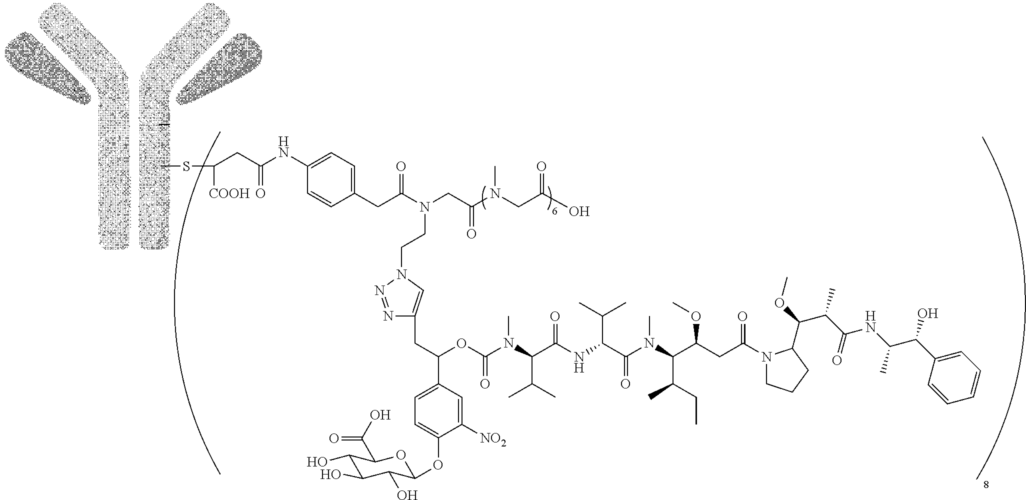 |
| Trastuzumab-MAL- phenyl-Ngly(triazole- glucuronideMMAE)- PSAR12 (ADC-PSAR12) | 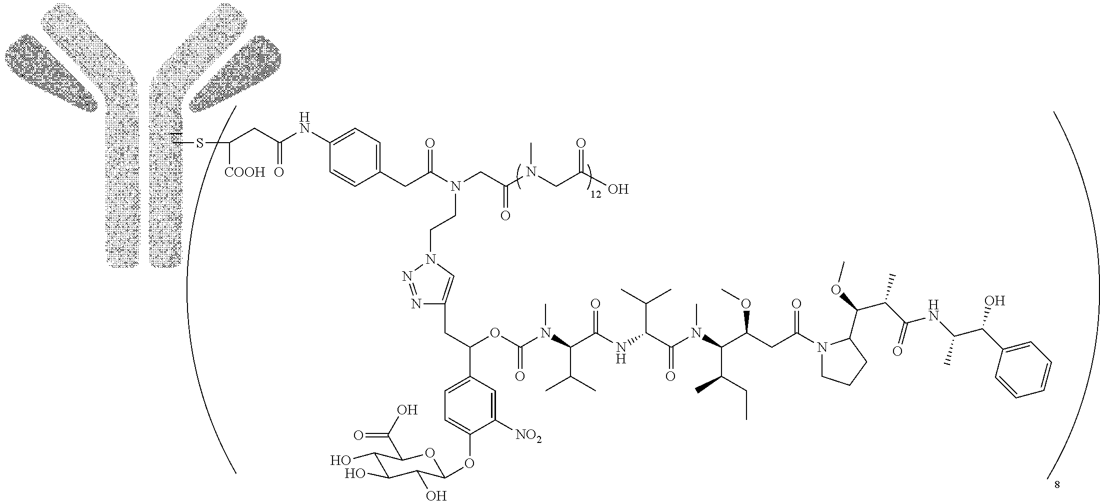 |
| Trastuzumab-MAL- phenyl-Ngly(triazole- val-cit-PAB-MMAE)- PSAR12 | 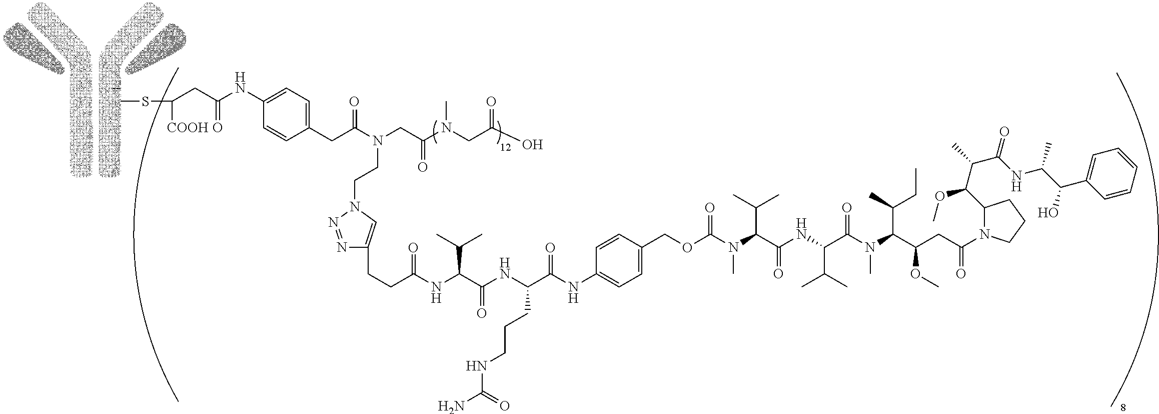 |
| Trastuzumab-MAL- phenyl-Ngly(triazole- glucuronideMMAE)- PSAR18 (ADC-PSAR18) | 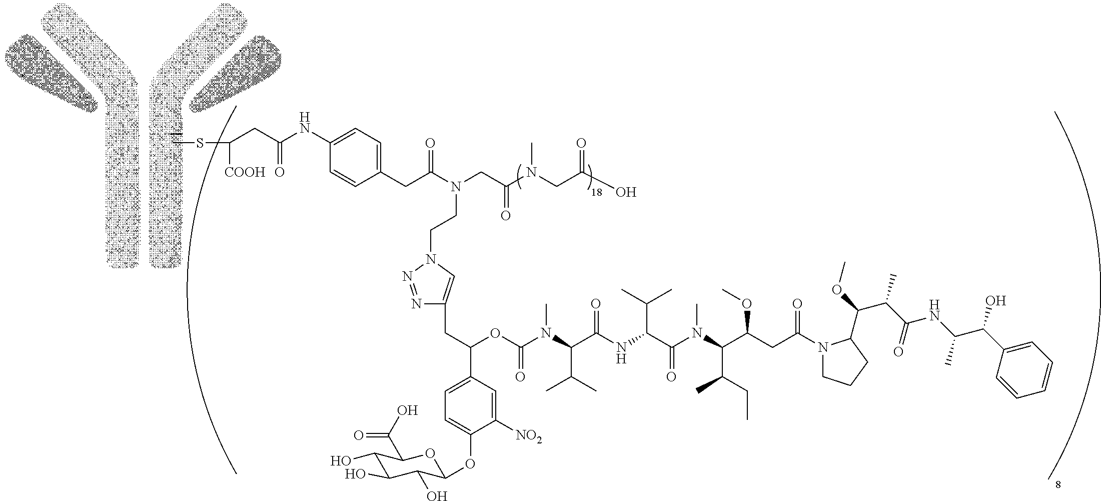 |
| Trastuzumab-MAL- phenyl-Ngly(triazole- SN38)-PSAR18 | 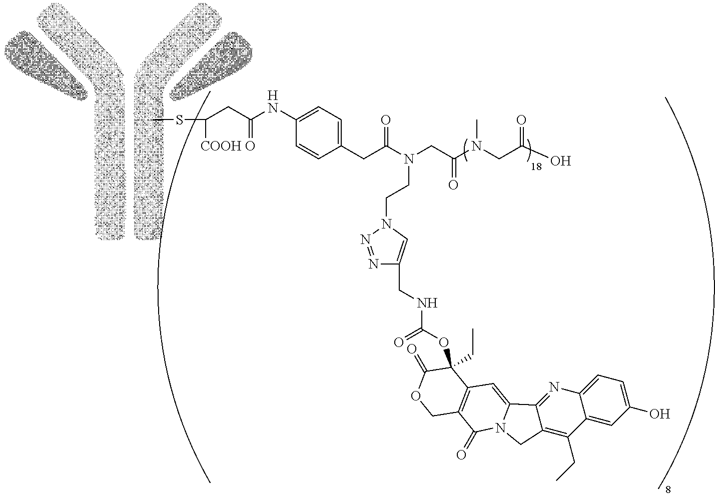 |
| Trastuzumab-MAL- phenyl-Ngly(triazole- glucuronide- Exatecan)-PSAR18 | 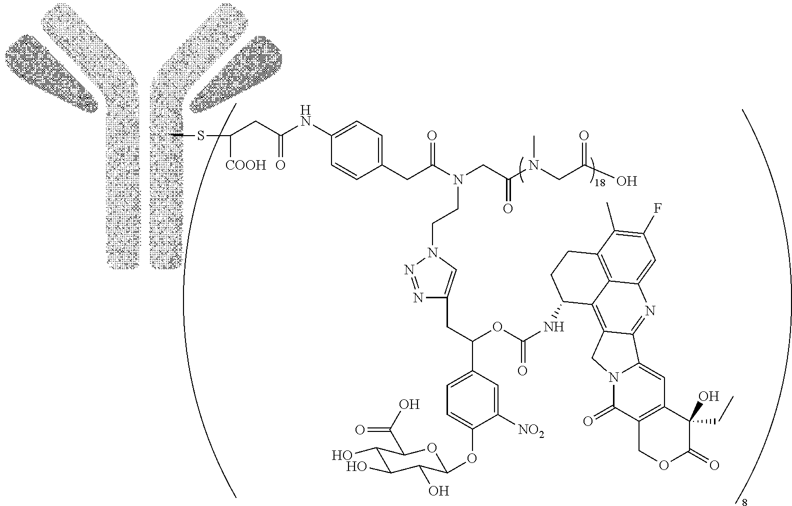 |
| Trastuzumab-MAL- phenyl-Ngly(triazole- PNU159682)- PSAR12 | 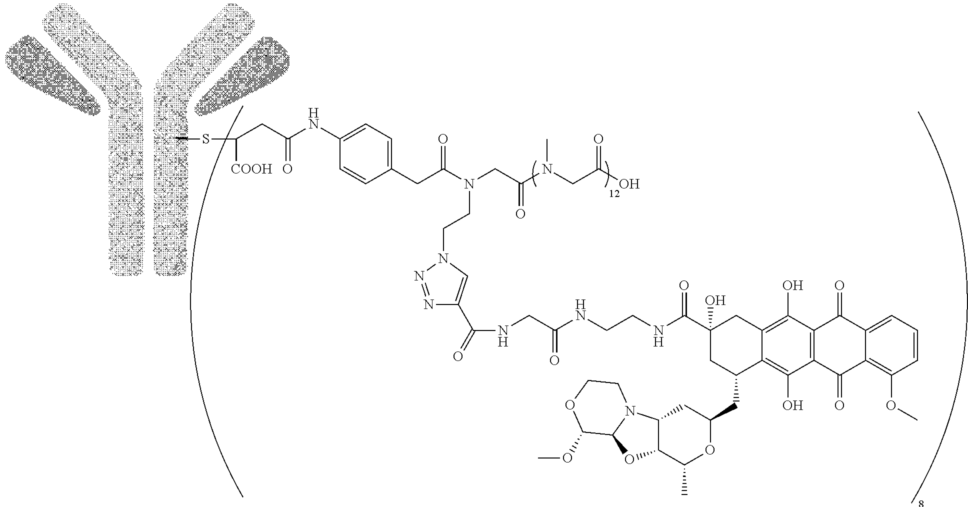 |
| Trastuzumab-MAL- phenyl-Ngly(triazole- PNU159682)- PSAR18 | 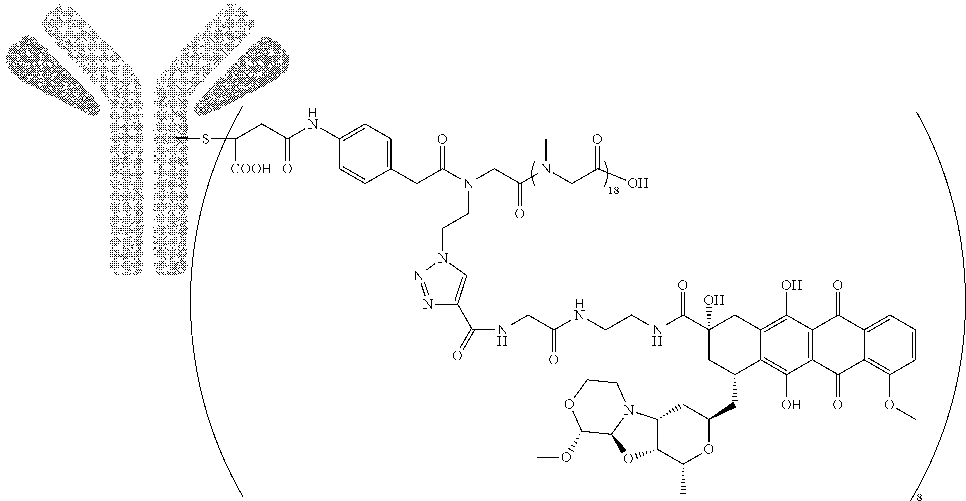 |
| Trastuzumab-MAL- phenyl-Ngly(triazole- glucuronideMMAE)- PSAR24 (ADC-PSAR24) | 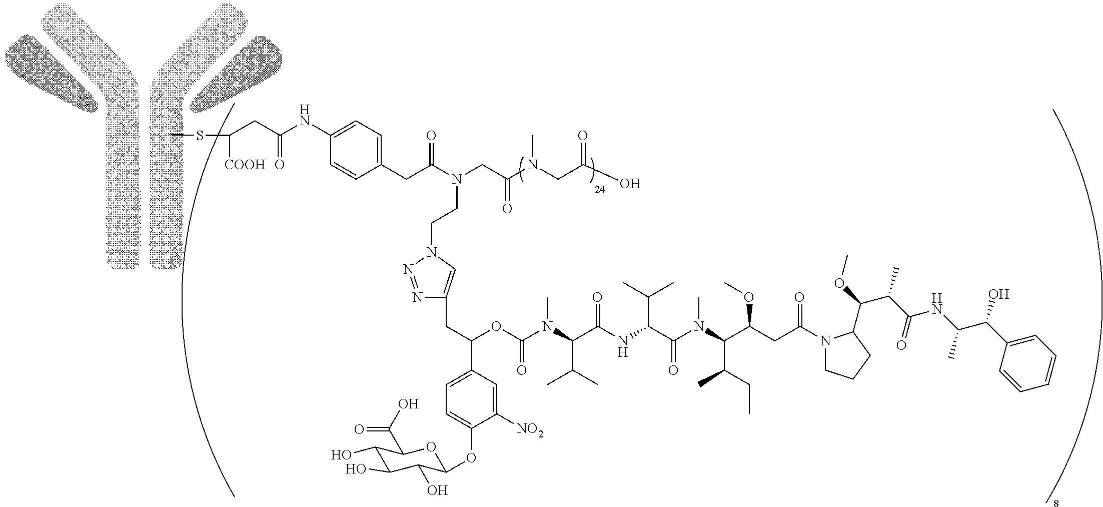 |
| CD19-MAL-phenyl- Ngly(triazole- glucuronideMMAE)- PSAR24 | 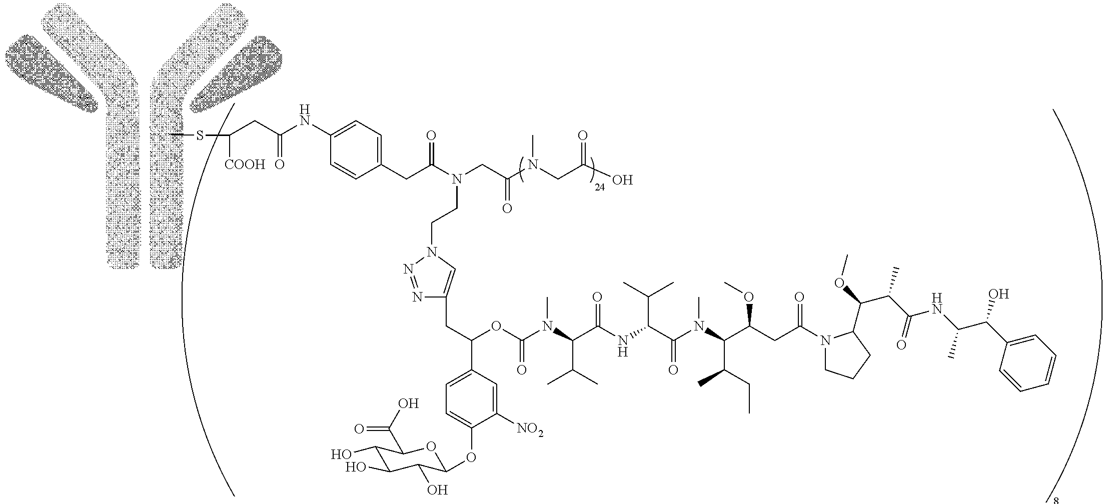 |
| CD22-MAL-phenyl- Ngly(triazole- glucuronideMMAE)- PSAR24 | 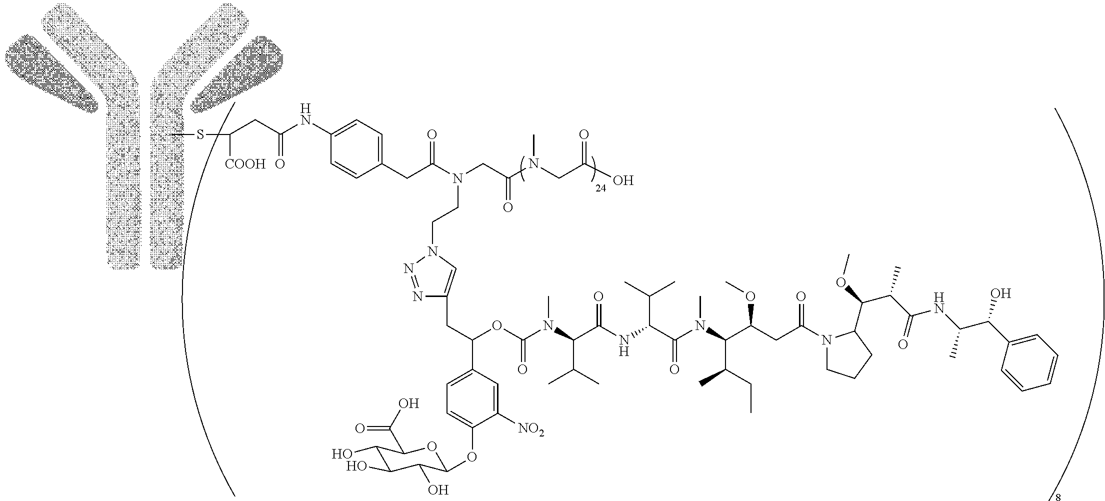 |
| Trastuzumab-MAL- phenyl-Ngly(triazole- glucuronideMMAE)- Ngly(triazole- glucoronideMMAE)- PSAR18 | 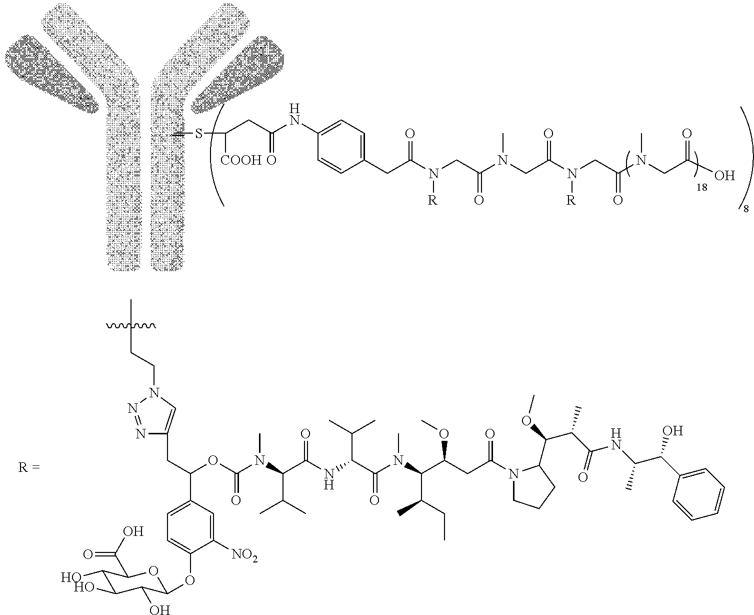 |
| Trastuzumab-MAL- phenyl-Ngly[triazole- glucuronide (MMAE)₂]-PSAR24 | 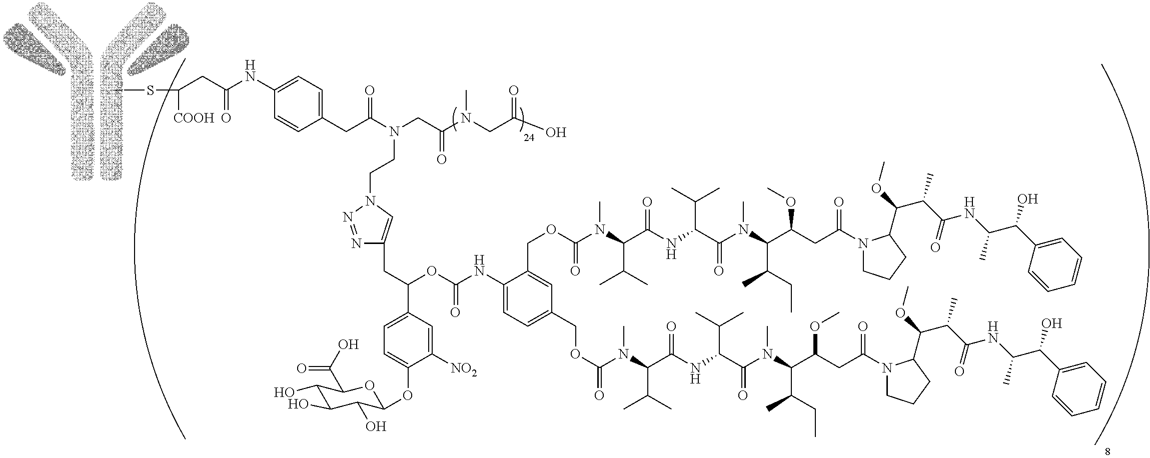 |
| Trastuzumab-MAL- phenyl-Ngly[triazole- galactoside(MMAE)₂]- PSAR24 | 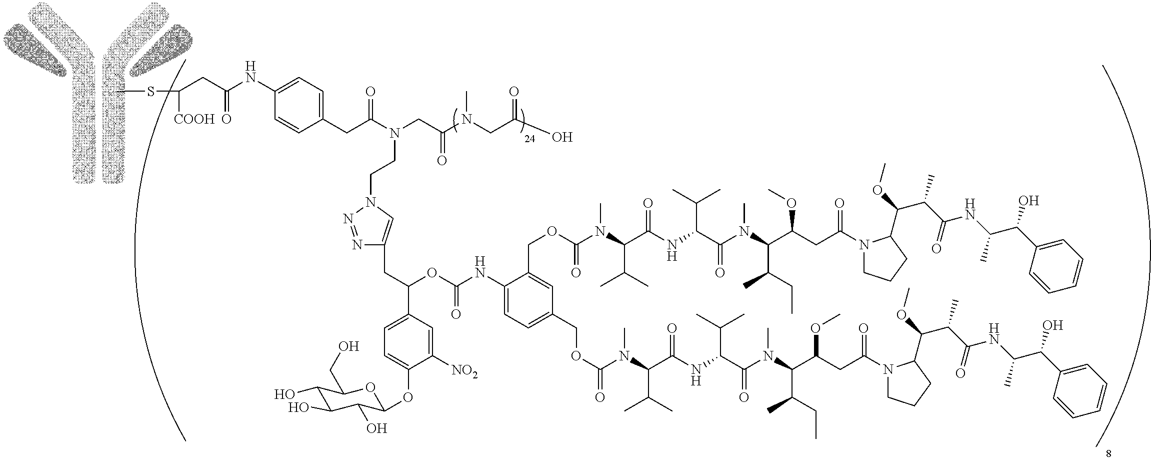 |
| Trastuzumab-MAL- phenyl-Ngly(triazole- SN38)-Ngly(triazole- SN38)-PSAR18 | 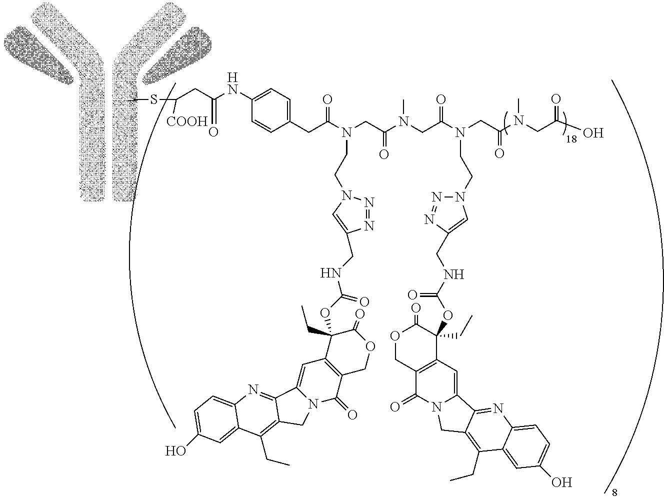 |
| Trastuzumab-MAL- phenyl-Ngly(triazole- glucuronideSN38)- Ngly(triazole- glucoronideSN38)- PSAR18 | 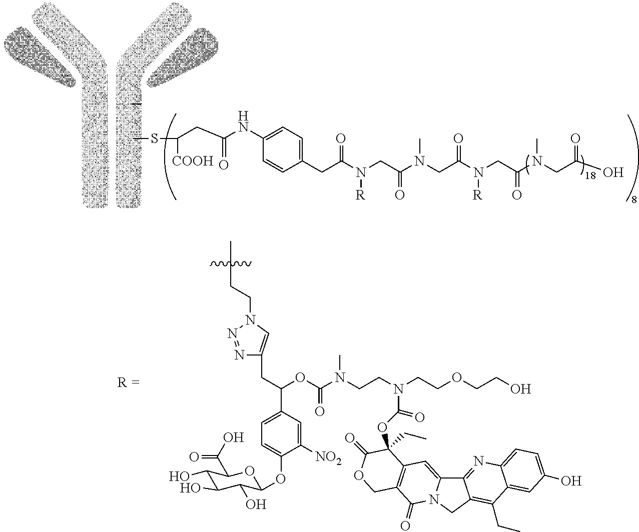 |
| Trastuzumab-MAL- phenyl-Ngly(triazole- glucuronideMMAE)- PEG12 (ADC-PEG12) | 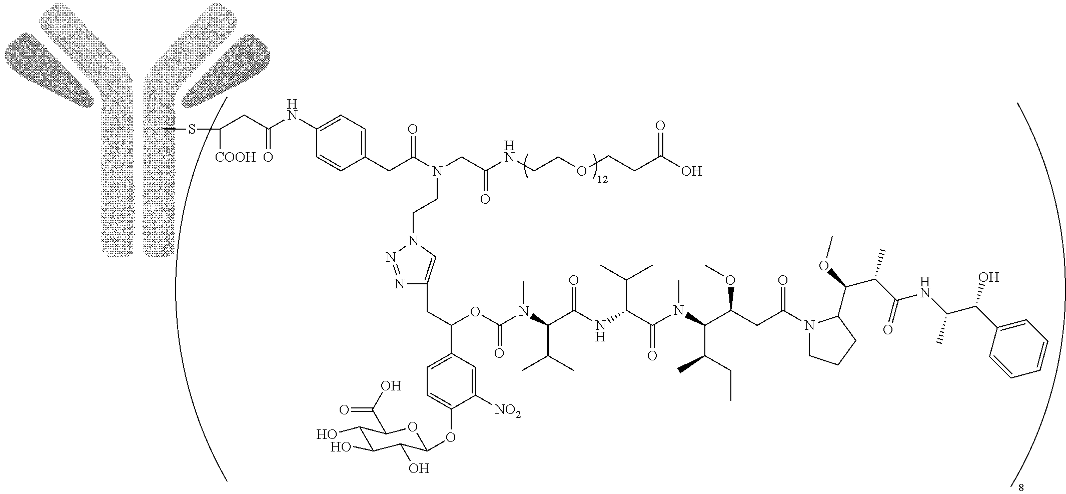 |
| Trastuzumab- glucoronideMMAE | 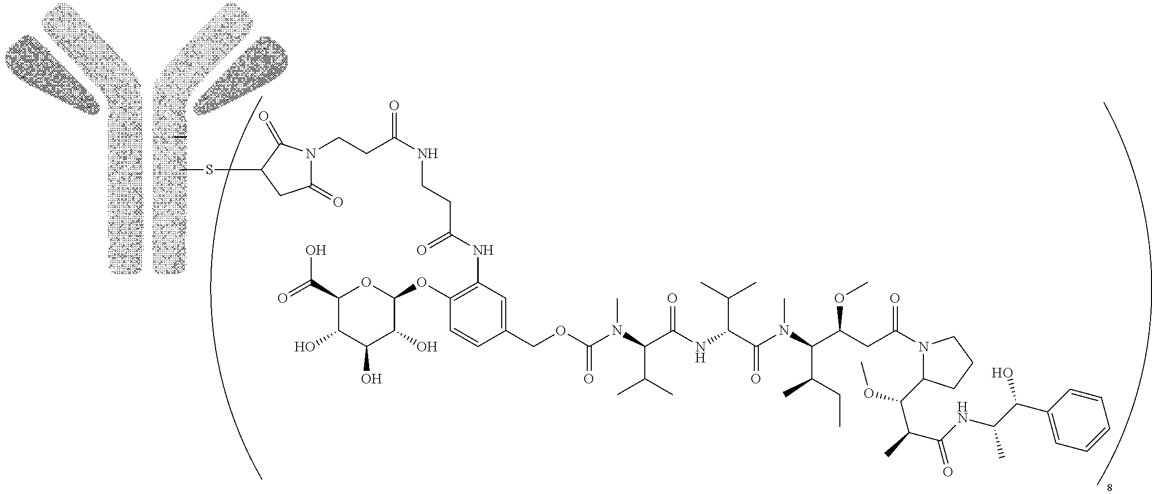 |
| Trastuzumab-MAL- phenyl-triazole- glucuronideMMAE (ADC-PSAR0) | 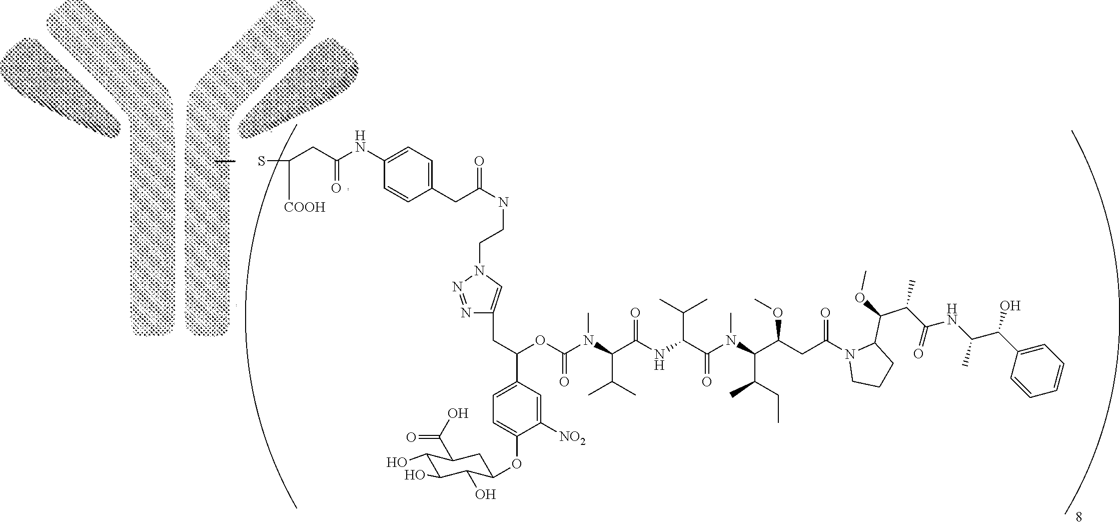 |
| Trastuzumab-MAL- phenyl-PSAR12- triazole- glucuronideMMAE (ADC-PSAR12L) | 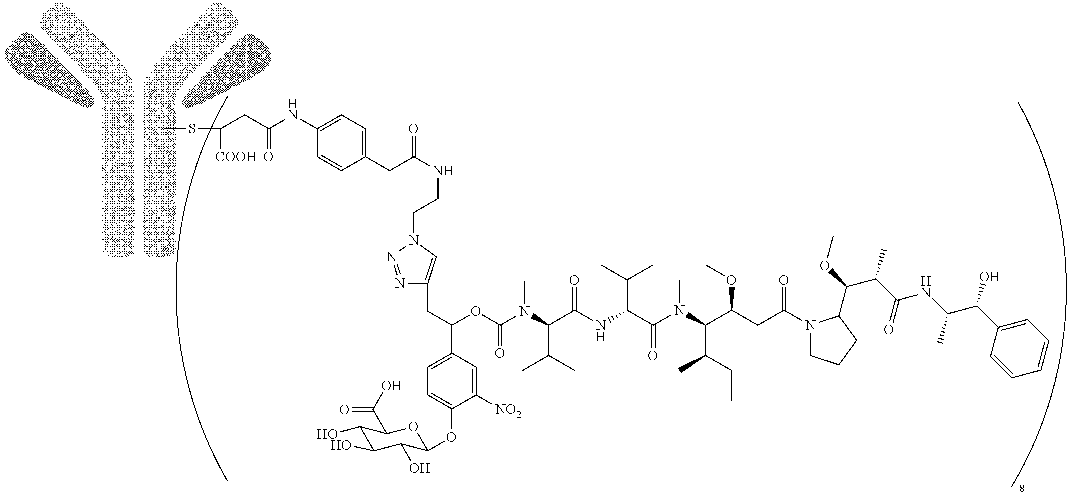 |
| Human albumin- MAL-phenyl- Ngly(triazole- glucuronideMMAE)- PSAR24 | 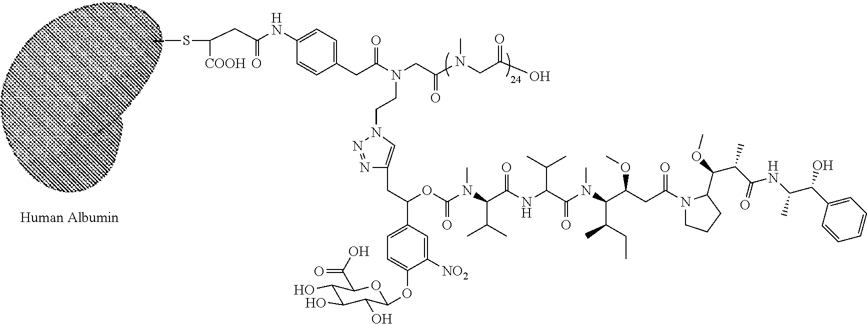 |

### 11.1) Preparation of Conjugates

### 11.1.1) Preparation of antibody-drug-conjugates

A solution of antibody (10 mg/mL in PBS 7.4+1 mM EDTA) was treated with 14 molar equivalent of tris(2-carboxyethyl)phosphine (TCEP) for 2 hours at 37° C. For maleimide-based coupling, the fully reduced antibody was buffer-exchanged with potassium phosphate 100 mM pH 7.4+1 mM EDTA by three rounds of dilution/centrifugation using Amicon 30K centrifugal filters device (Merck Millipore). 10-12 molar equivalents of drug-linker (from a 12 mM DMSO stock solution) was added to the antibody (residual DMSO <10% v/v). The solution was incubated 30 min at room temperature. For bromoacetamide-based coupling the fully reduced antibody was buffer-exchanged with borate buffer 50 mM pH 8.1+1 mM EDTA and conjugation was realized using 16 molar equivalents of drug-linker during 24 hours at 37° C. in the dark. The conjugate was buffer-exchanged/purified with PBS 7.4 by four rounds of dilution/centrifugation using Amicon 30K centrifugal filters device. Alternatively, conjugates were buffer-exchanged/purified using PD MiniTrap G-25 columns (GE Healthcare) and were sterile-filtered (0.20 μm PES filter). Conjugates incorporating the self-hydrolysable maleimide (MAL-phenyl) group were incubated at 5 mg/mL in PBS 7.4 at 37° C. for 48h to ensure complete hydrolysis of the succinimidyl moiety. Final protein concentration was assessed spectrophotometrically at 280 nm using a Colibri microvolume spectrometer device (Titertek Berthold).

### 11.1.2) Preparation of Human Albumin-Drug-Conjugates

To a solution of human albumin (10 mg/mL in potassium phosphate 100 mM pH 7.4+1 mM EDTA) was added 2 molar equivalents of drug-linker (from a 12 mM DMSO stock solution). Residual DMSO was <10% (v/v). The solution was incubated for 4 hours at room temperature. The conjugate was buffer-exchanged/purified with PBS 7.4 by four rounds of dilution/centrifugation using Amicon 30K centrifugal filters device. Alternatively, conjugates were buffer-exchanged/purified using PD MiniTrap G-25 columns (GE Healthcare) and were sterile-filtered (0.20 μm PES filter). Conjugates were incubated at 5 mg/mL in PBS 7.4 at 37° C. for 48h to ensure complete hydrolysis of the succinimidyl moiety. Final protein concentration was assessed spectrophotometrically at 280 nm using a Colibri microvolume spectrometer device (Titertek Berthold).

### 11.2) Characterization of Conjugates

The resulting conjugates were characterized as follows:

### Reverse Phase Liquid Chromatography-Mass Spectrometry (RPLC-MS)

Denaturing RPLC-QToF analysis was performed using the UHPLC method 5 described above. Briefly, conjugates were eluted on an Agilent PLRP-S 1000 Å 2.1×150 mm 8 μm (80° C.) using a mobile phase gradient of water/acetonitrile+0.1% formic acid (0.4 mL/min) and detected using a Bruker Impact II™ Q-ToF mass spectrometer scanning the 500-3500 m/z range (ESIJ. Data were deconvoluted using the MaxEnt algorithm included in the Bruker Compass® software.

### Size Exclusion Chromatography (SEC)

SEC was performed on an Agilent 1050 HPLC system having an extra-column volume below 15 μL (equipped with short sections of 0.12 mm internal diameter peek tubing and a micro-volume UV flow cell). Column was a Waters Acquity UPLC® Protein BEH SEC 200Å 4.6×150 mm 1.7 μm (maintained at room temperature) or an Agilent AdvanceBio SEC 300Å 4.6×150 mm 2.7 μm (maintained at room temperature). Mobile phase was 100 mM sodium phosphate and 200 mM sodium chloride (pH 6.8). 10% acetonitrile (v/v) was added to the mobile phase to minimize secondary hydrophobic interactions with the stationary phase and prevent bacterial growth. Flow rate was 0.35 mL/min. UV detection was monitored at 280 nm.

### Hydrophobic Interaction Chromatography (HIC)

Hydrophobic interaction chromatography (HIC) was performed on an Agilent 1050 HPLC system. Column was a Tosoh TSK-GEL BUTYL-NPR 4.6×35 mm 2.5 μm (25° C.). Mobile phase A was 1.5 M (NH₄)₂SO₄+25 mM potassium phosphate pH 7.0. Mobile phase B was 25 mM potassium phosphate pH 7.0+15% isopropanol (v/v). Linear gradient was 0% B to 100% B in 10 min, followed by a 3 min hold at 100% B. Flow rate was 0.75 mL/min. UV detection was monitored at 220 and 280 nm.

### 11.3) Overview of Conjugates Characterization

Conjugates exhibited one LC-1d (light chain with 1 drug-linker attached) and one HC-3d (heavy chain with 3 drug-linkers attached) absorbance peaks on their denaturing RPLC chromatogram (DAR8 conjugates). For mass spectrometry analysis of the heavy chain, the major glycoform was reported (G0F for trastuzumab). Conjugates exhibited a single absorbance peak on their HIC chromatogram.

### Trastuzumab-Glu(glucuronideMMAE)-CH₂—CH₂-PSAR6 (DAR8)

Deconvoluted LC-1d Calc: 25416; Obs: 25417/Deconvoluted HC-3d Calc: 56529; Obs: 56528
Monomeric purity: 97.2%
HIC retention time: 8.8 min

### Trastuzumab-Glu(glucuronideMMAE)-CH₂—CH₂-PSAR12 (DAR8)

Deconvoluted LC-1d Calc: 25844; Obs: 25844/Deconvoluted HC-3d Calc: 57805; Obs: 57805
Monomeric purity: 99.0%
HIC retention time: 8.8 min

### Trastuzumab-Glu(glucuronideMMAE)-CH₂—CH₂-PSAR18 (DAR8)

Deconvoluted LC-1d Calc: 26270; Obs: 26270/Deconvoluted HC-3d Calc: 59086; Obs: 59086
Monomeric purity: 96.5%
HIC retention time: 8.8 min

### Trastuzumab-Lys(glucuronideMMAE)-PSAR6 (DAR8)

Deconvoluted LC-1d Calc: 25360; Obs: 25360/Deconvoluted HC-3d Calc: 56352; Obs: 56353
Monomeric purity: 99+%
HIC retention time: 7.6 min

### Trastuzumab-Lys(glucuronideMMAE)-PSAR12 (DAR8)

Deconvoluted LC-1d Calc: 25786; Obs: 25786/Deconvoluted HC-3d Calc: 57634; Obs: 57632
Monomeric purity: 99+%
HIC retention time: 7.5 min

### Trastuzumab-BAC-Ngly(triazole-glucuronideMMAE)-PSAR12 (DAR8)

Deconvoluted LC-1d Calc: 25661; Obs: 25662/Deconvoluted HC-3d Calc: 57264; Obs: 57262
Monomeric purity: 99+%
HIC retention time: 7.0 min (DAR8 conjugate). This ADC is an heterogeneous mixture containing ˜20% of DAR6; ˜20% of DAR7 and ˜60% of DAR8 conjugates, as observed on the HIC chromatogram.

### Trastuzumab-MAL-phenyl-Ngly(triazole-glucuronideMMAE)-PSAR6 (DAR8) (=ADC-PSAR6)

Deconvoluted LC-1d Calc: 25368; Obs: 25368/Deconvoluted HC-3d Calc: 56382; Obs: 56380
Monomeric purity: 99+%
HIC retention time: 7.5 min

### Trastuzumab-MAL-phenyl-Ngly(triazole-glucuronideMMAE)-PSAR12 (DAR8) (=ADC-PSAR12)

Deconvoluted LC-1d Calc: 25794; Obs: 25794/Deconvoluted HC-3d Calc: 57660; Obs: 57660
Monomeric purity: 99+%
HIC retention time: 7.1 min

### Trastuzumab-MAL-phenyl-Ngly(triazole-val-cit-PAB-MMAE)-PSAR12 (DAR8)

Deconvoluted LC-1d Calc: 25871; Obs: 25870/Deconvoluted HC-3d Calc: 57889; Obs: 57888
Monomeric purity: 99+%
HIC retention time: 9.2 min

### Trastuzumab-MAL-phenyl-Ngly(triazole-glucuronideMMAE)-PSAR18 (DAR8) (=ADC-PSAR18)

Deconvoluted LC-1d Calc: 26221; Obs: 26221/Deconvoluted HC-3d Calc: 58939; Obs: 58939
Monomeric purity: 99+%
HIC retention time: 6.8 min

### Trastuzumab-MAL-phenyl-Ngly(triazole-SN38)-PSAR18 (DAR8)

Deconvoluted LC-1d Calc: 25567; Obs: 25567/Deconvoluted HC-3d Calc: 56979; Obs: 56977
Monomeric purity: 99+%
HIC retention time: 5.1 min

### Trastuzumab-MAL-phenyl-Ngly(triazole-glucuronide-Exatecan)-PSAR18 (DAR8)

Deconvoluted LC-1d Calc: 25938; Obs: 25937/Deconvoluted HC-3d Calc: 58092; Obs: 58089
Monomeric purity: 99+%
HIC retention time: 5.7 min

### Trastuzumab-MAL-phenyl-Ngly(triazole-PNU159682)-PSAR12 (DAR8)

Deconvoluted LC-1d Calc: 25446; Obs: 25446/Deconvoluted HC-3d Calc: 56614; Obs: 56612
Monomeric purity: 99+%
HIC retention time: 5.2 min

### Trastuzumab-MAL-phenyl-Ngly(triazole-PNU159682)-PSAR18 (DAR8)

Deconvoluted LC-1d Calc: 25872; Obs: 25872/Deconvoluted HC-3d Calc: 57894; Obs: 57892
Monomeric purity: 99+%
HIC retention time: 5.1 min

### Trastuzumab-MAL-phenyl-Ngly(triazole-glucuronideMMAE)-PSAR24 (DAR8) (=ADC-PSAR24)

Deconvoluted LC-1d Calc: 26647; Obs: 26674/Deconvoluted HC-3d Calc: 60218; Obs: 60218
Monomeric purity: 99+%
HIC retention time: 6.7 min

### CD19-MAL-phenyl-Ngly(triazole-glucuronideMMAE)-PSAR24 (DAR8)

Deconvoluted LC-1d Calc: 27347; Obs: 27347/Deconvoluted HC-3d Calc: 60137; Obs: 60132
Monomeric purity: 92.6%
HIC retention time: 6.8 min

### CD22-MAL-phenyl-Ngly(triazole-glucuronideMMAE)-PSAR24 (DAR8)

Deconvoluted LC-1d Calc: 27341; Obs: 27341/Deconvoluted HC-3d Calc: 60314; Obs: 60311
Monomeric purity: 97.4%
HIC retention time: 6.8 min

### Trastuzumab-MAL-phenyl-Ngly(triazole-glucuronideMMAE)-Ngly(triazole-glucuronideMMAE)-PSAR18 (DAR16)

Deconvoluted LC-1d Calc: 27544; Obs: 27545/Deconvoluted HC-3d Calc: 62910; Obs: 62907
Monomeric purity: 98.5%
HIC retention time: 8.7 min

### Trastuzumab-MAL-phenyl-Ngly[triazole-glucuronide(MMAE)₂]-PSAR24 (DAR16)

Deconvoluted LC-1d Calc: 27569; Obs: 27570/Deconvoluted HC-3d Calc: 62985; Obs: 62983
Monomeric purity: 98.2%
HIC retention time: 9.9 min

### Trastuzumab-MAL-phenyl-Ngly[triazole-galactoside(MMAE)₂₁-PSAR24 (DAR16)

Deconvoluted LC-1d Calc: 27555; Obs: 27554/Deconvoluted HC-3d Calc: 62943; Obs: 62940
Monomeric purity: 99+%
HIC retention time: 10.2 min

### Trastuzumab-MAL-phenyl-Ngly(triazole-SN38)-Ngly(triazole-SN38)-PSAR18 (DAR16)

Deconvoluted LC-1d Calc: 26237; Obs: 26237/Deconvoluted HC-3d Calc: 58989; Obs: 58987
Monomeric purity: 99+%
HIC retention time: 6.6 min

### Trastuzumab-MAL-phenyl-Ngly(triazole-glucuronideSN38)-Ngly(triazole-glucuronideSN38)-PSAR18 (DAR16)

Deconvoluted LC-1d Calc: 27270; Obs: 27270/Deconvoluted HC-3d Calc: 62086; Obs: 62087
Monomeric purity: 99+%
HIC retention time: 5.7 min

### Trastuzumab-MAL-phenyl-Ngly(triazole-glucuronideMMAE)-PEG12 (DAR8) (=ADC-PEG12)

Deconvoluted LC-1d Calc: 25541; Obs: 25541/Deconvoluted HC-3d Calc: 56901; Obs: 56900
Monomeric purity: 99+%
HIC retention time: 7.4 min

### Trastuzumab-glucuronideMMAE (DAR8)

Deconvoluted LC-1d Calc: 24721; Obs: 24720/Deconvoluted HC-3d Calc: 54439; Obs: 54438
Monomeric purity: 95.2%
HIC retention time: 9.2 min

### Trastuzumab-MAL-phenyl-triazole-glucuronideMMAE (DAR8) (=ADC-PSAR0)

Deconvoluted LC-1d Calc: 24884; Obs: 24884/Deconvoluted HC-3d Calc: 54926; Obs: 54926
Monomeric purity: 98.5%
HIC retention time: 8.4 min

### Trastuzumab-MAL-phenyl-PSAR12-triazole-glucuronideMMAE (DAR8) (=ADC-PSAR12L)

Deconvoluted LC-1d Calc: 25793; Obs: 25793/Deconvoluted HC-3d Calc: 57657; Obs: 57657
Monomeric purity: 99+%
HIC retention time: 8.5 min

### Human albumin-MAL-phenyl-Ngly(triazole-glucuronideMMAE)-PSAR24 (DAR1)

Deconvoluted Calc: 69765; Obs: 69645
Monomeric purity: 90.8%
HIC retention time: 3.9 min

### Trastuzumab

Deconvoluted LC Obs: 23439/Deconvoluted HC Obs: 50595
Monomeric purity: 99+%
HIC retention time: 4.7 min

### Anti-CD19 antibody

Deconvoluted LC Obs: 24139/Deconvoluted HC Obs: 50517
Monomeric purity: 93.2%
HIC retention time: 4.7 min

### Anti-CD22 antibody

Deconvoluted LC Obs: 24133/Deconvoluted HC Obs: 50692
Monomeric purity: 99+%
HIC retention time: 4.8 min

### Human Albumin

Deconvoluted Obs: 66556
Monomeric purity: 92.4%
HIC retention time: 2.5 min

### Example 12: Hydrophobic Interaction Chromatography (HIC) Profiles of Non-Polysarcosine-Based Antibody-Dru2-Conjugate (ADC-PSAR0), Polysarcosine-Based Antibody-Drug-Conjugate with an Orthogonal Configuration (ADC-PSAR12) and Polysarcosine-Based Antibody-Drug-Conjugate with a Linear Configuration (ADC-PSAR12L)

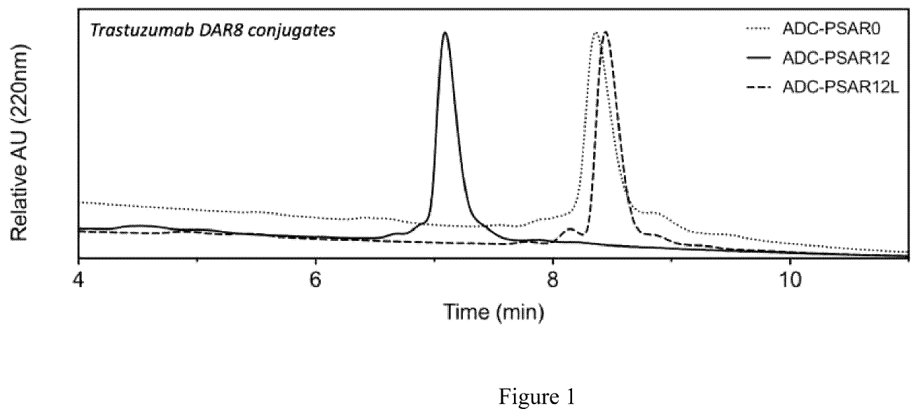

The relative exposure of the conjugated payload to bulk solvent and the apparent hydrophobicity of trastuzumab-based DAR8 ADC was assessed by hydrophobic interaction chromatography (HIC) on a Tosoh TSK-GEL BUTYL-NPR column, following the method described in Example 11. The results are shown in FIG. 1. Polysarcosine, when grafted in parallel (i.e. orthogonal) orientation in relation to the drug unit, provides efficient hydrophobicity masking properties and reduced apparent hydrophobicity of the conjugate (ADC-PSAR12). When polysarcosine is in a linear (i.e. serial) configuration however, no reduction of the apparent hydrophobicity of the conjugate is observed (ADC-PSAR12L).

### Example 13: Hydrophobic Interaction Chromatography (HIC) Profiles of Polysarcosine- and Polyethyleneglycol-Based Antibody-Drug-Conjugates

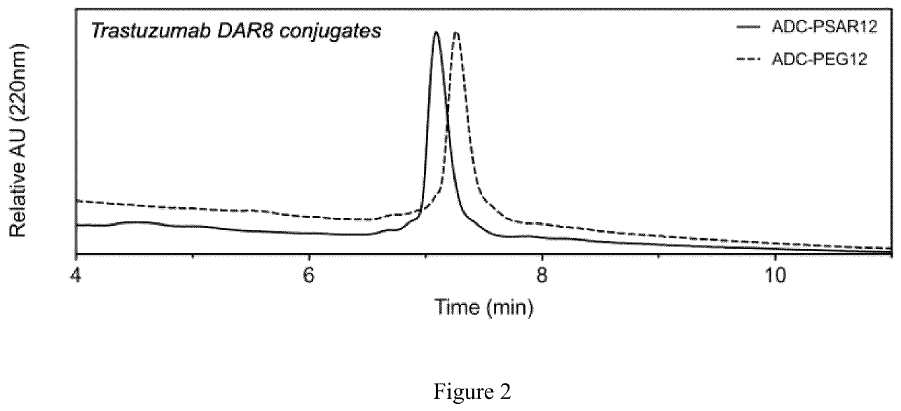

The relative exposure of the conjugated payload to bulk solvent and the apparent hydrophobicity of trastuzumab-based DAR8 ADC was assessed by hydrophobic interaction chromatography (HIC) on a Tosoh TSK-GEL BUTYL-NPR column, following the method described in Example 11. The results are shown in FIG. 2. At equal length (n=12 monomer units), polysarcosine provides better hydrophobicity masking properties than polyethyleneglycol (lower retention time).

### Example 14: Pharmacokinetic Profile (Total Antibody Concentration Over Time) in Mice Following a Single Intravenous 3 mg/kg Dose of Non-Polysarcosine-Based Antibody-Drug-Conjugate (ADC-PSAR0) and Polysarcosine-Based Antibody-Drug-Conjugate (ADC-PSAR12)

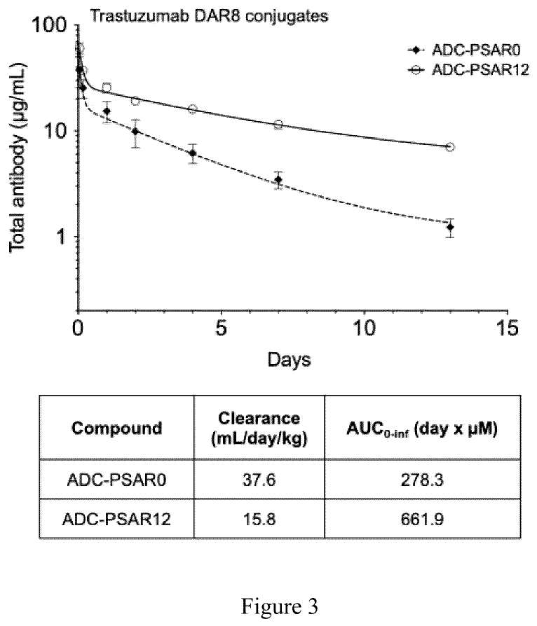

ADCs were injected at 3 mg/kg in male SCID mice (4-6 weeks old) via the tail vein (five animals per dose group, randomly assigned). Blood was drawn into citrate tubes via retro-orbital bleeding at various time points and processed to plasma. Total ADC concentration was assessed using a human IgG ELISA kit (Stemcell™ Technologies) according to the manufacturer's protocol. Standard curves of Trastuzumab were used for quantification. Pharmacokinetics parameters (clearance and AUC) were calculated by non-compartmental analysis using Microsoft® Excel® software incorporating PK functions (add-in developed by Usansky et al., Department of Pharmacokinetics and Drug Metabolism, Allergan, Irvine, USA). The results are shown in FIG. 3. ADC comprising polysarcosine exhibit favorable pharmacokinetics when compared to ADC without polysarcosine.

### Example 15: Tumor Volume (Mm³) and Survival Curves in a BT-474 Breast Cancer Xenograft Model Dosed Once Intravenously with 3 mg/kg of Non-Polysarcosine-Based ADC (ADC-PSAR0) and Polysarcosine-Based ADC (ADC-PSAR12)

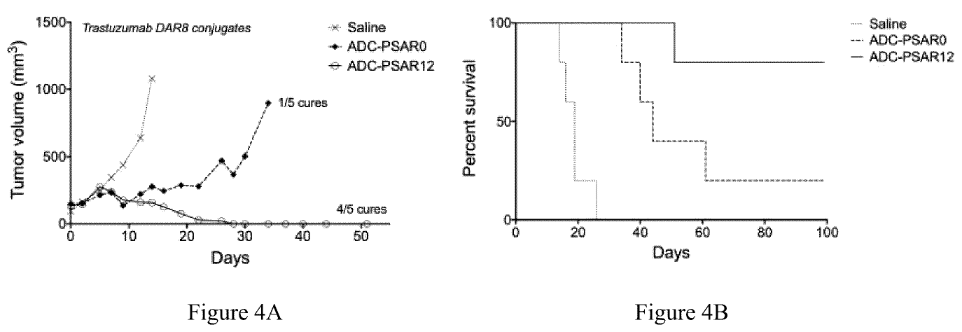

BT-474 breast cancer cells were implanted subcutaneously in female SCID mice (4 weeks old). ADCs from above Example 14 were dosed once intravenously at a 3 mg/kg dose when tumors had grown to approximately 150 mm³ (Day 20, 5 animals per group, assigned to minimize differences in initial tumor volumes between groups). The results are shown in FIGS. 4A and 4B. Tumor volume was measured every 3-5 days by a caliper device and was calculated using the formula (L×W²)/2. Mice were sacrificed when the tumor volume exceeded 1000 mm³. ADC comprising polysarcosine have improved in vivo activity when compared to ADC without polysarcosine. No significant body-weight change was observed in treated mice.

### Example 16: Pharmacokinetic Profile (Total Antibody Concentration Over Time) in Mice Following a Single Intravenous 3 mg/kg Dose of Polysarcosine-Based Antibody-Drug-Conjugate (ADC-PSAR12) and Poly(Ethyleneglycol)-Based Antibody-Drug-Conjugate (ADC-PEG12)

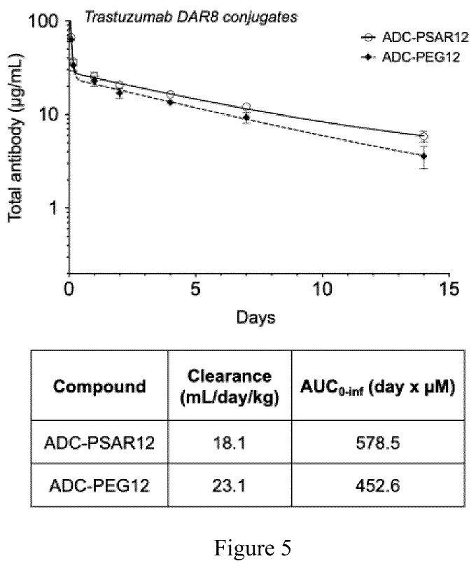

Experiment was conducted according to the procedure described in Example 14, in male CD-1 mice (4-6 weeks old). The results are shown in FIG. 5. ADC comprising polysarcosine have improved pharmacokinetics parameters when compared to ADC comprising poly(ethyleneglycol).

### Example 17: Tumor Volume (Mm³) in a BT-474 Breast Cancer Xenograft Model Dosed Once Intravenously with 2.5 mg/kg of Polysarcosine-Based Antibody-Drug Conjugates Having Different PSAR Lengths in an Orthogonal Orientation (ADC-PSAR6, ADC-PSAR12, ADC-PSAR18, ADC-PSAR24); Orthogonal Poly(Ethyleneglycol)-Based Antibody-Drug Conjugate (ADC-PEG12) and Linear Polysarcosine-Based Antibody-Drug Conjugates (ADC-PSAR12L)

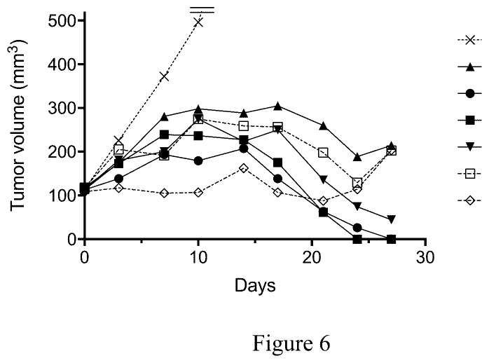

Experiment was conducted as described in Example 15. BT-474 breast cancer cells were implanted subcutaneously in female SCID mice (4 weeks old). ADCs were dosed once intravenously at a 2.5 mg/kg dose when tumors had grown to approximately 150 mm³ (Day 13, 6 animals per group, assigned to minimize differences in initial tumor volumes between groups). The results are shown in FIG. 6. No significant body-weight change was observed in treated mice.

1. A Ligand-Drug-Conjugate compound (LDC) having the following formula (XV) 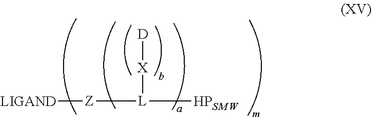
wherein
L is an orthogonal connector that allows for (HP_{SMW}) to be in an orthogonal orientation with respect to (X-D),
HP_{SMW} results from covalent binding to said orthogonal connector L, of a single molecular weight homopolymer having formula (I) 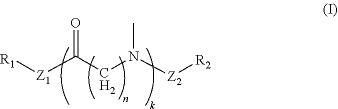
wherein
R₁ and R₂ are different, and
one of R₁ and R₂ is H or an inert group, the other one of R₁ and R₂ being a functionalized reactive group, said group being reactive for covalently binding a bindable group, in such reaction conditions that the inert group is non-reactive,
Z₁ and Z₂, identical or different, are optional spacers, and
n is 1 or more and k is 2 or more;
D is a cytotoxic drug,
X is an optional cleavable moiety for releasing D,
Z is an optional spacer, and
a is 1 or more, b is 1 or more and m is 1 or more.

2. The LDC compound of claim 1, wherein said single molecular weight homopolymer is polysarcosine.

3. The LDC compound of claim 2, wherein the HP_{SMW} results from covalent binding to the orthogonal connector L, of a single molecular weight homopolymer having formula (II) 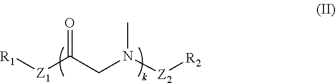
wherein
R₁ R₂, Z₁ and Z₂ are as defined in claim 1, and
k is 2 to 100, preferably 2 to 50.

4. The LDC compound of claim 1, wherein R₁ or R₂ is a functionalized reactive group and is selected from the following groups:
carboxylic acid group,
amino groups NRR″ wherein R and R″ are independently selected from H, (C₁-C₆) alkyl optionally interrupted by at least one heteroatom selected among O, N and S,
hydroxyl group,
halogen atoms,
hydrazine (—NH₂—NH₂) group,
nitro group,
hydroxylamine group,
azido group,
(C₂-C₆) alkynyl group,
(C₂-C₆) alkenyl group,
thiol group,
activated ester groups such as N-hydroxysuccinimide ester, perfluorinated esters, nitrophenyl esters, aza-benzotriazole and benzotriazole activated esters, acylureas,
boronic acid —B(OR″ ″)₂ groups, wherein R″ ″ is a hydrogen atom or a C₁-C₆ alkyl group,
thiol-reactive groups such as maleimide, halomaleimides, haloacetyls, pyridyl disulfides,
mesylate group,
tosylate group,
triflate group,
aldehyde group,
isocyanate or isothiocyanate group,
chlorosulfonyl group,
acrylate group.

5. The LDC compound of claim 2, wherein said LIGAND is selected from the group consisting of a polypeptide, a protein, an antibody and an antibody fragment.

6. The LDC compound of claim 2, wherein D is selected from the group consisting of a bioactive molecule, a therapeutic molecule such as an anticancer drug, an imaging agent and a fluorophore.

7. The LDC compound of claim 2, wherein L is one or more natural or non-natural aminoacids.

8. The LDC compound of claim 2, wherein L is selected from glutamic acid, lysine and glycine.

9. The LDC compound of claim 2, wherein X is selected form
one or more natural or non-natural amino acids,
a sugar moiety linked via an oxygen glycosidic bond to a self immolative group,
a disulfide linker, and
an acid-labile linker that is hydrolysable in the lysosome.

10. The LDC compound of claim 2, wherein X is selected form
one or more natural or non-natural amino acids, and
a sugar moiety linked via an oxygen glycosidic bond to a self immolative group.

11. The LDC compound of claim 2, wherein Z is selected from alkylene, heteroalkylene; alkoxy; polyether; one or more natural or non-natural aminoacids; C₃-C₈ heterocyclo; C₃-C₈ carbocyclo; arylene; and any combination thereof.

12. The LDC compound of claim 11, wherein Z is of formula (XVII), (XVIII), (XIX), (XX), (XXI) or (XXII), 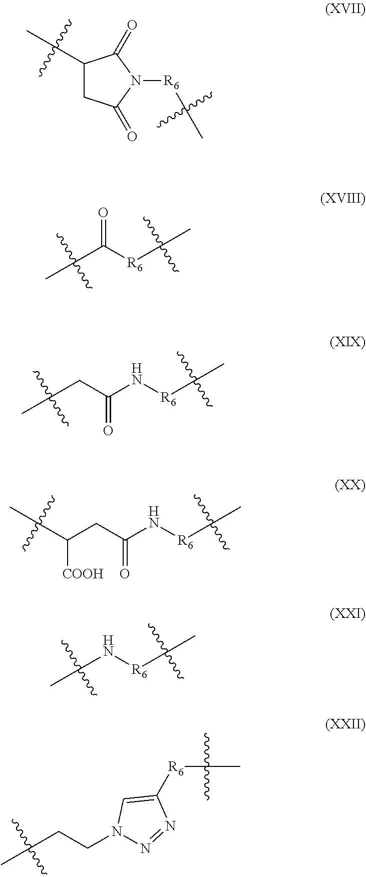
wherein the wavy bonds represent the attachment points and R₆ is —C₁-C₁₀ alkylene-, —C₁-C₁₀ heteroalkylene-, —C₁-C₁₀ alkylene-C(═O)—, —C₁-C₁₀ heteroalkylene-C(═O)—, -arylene-C₁-C₁₀ alkylene-C(═O)—, -arylene-C₁-C₁₀ alkylene-O—C(═O)—, and
any of the R₆ group is optionally substituted with one or more ═0.

13. An intermediate compound having formula (XVI) 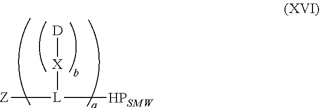
wherein
L is an orthogonal connector,
HP_{SMW} results from covalent binding to said orthogonal connector L, of a single molecular weight homopolymer having formula (I) 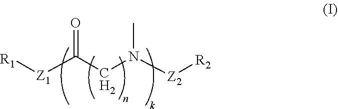
wherein
R₁ and R₂ are different, and
one of R₁ and R₂ is H or an inert group, the other one of R₁ and R₂ being a functionalized reactive group, said group being reactive for covalently binding a bindable group, in such reaction conditions that the inert group is non-reactive,
Z₁ and Z₂, identical or different, are optional spacers, and
n is 1 or more and k is 2 or more;
D is a cytotoxic drug,
X is an optional cleavable moiety for releasing D,
Z is an optional spacer, and
a is 1 or more and b is 0, 1 or more.

14. The intermediate compound of claim 13, wherein said single molecular weight homopolymer is polysarcosine.

15. The LDC of, for use as a medicament.

16. A compound having formula (XXIII) 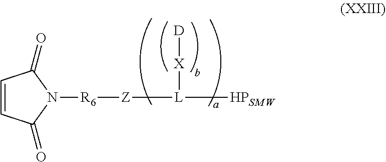
wherein
R₆ is —C₁-C₁₀ alkylene-, —C₁-C₁₀ heteroalkylene-, —C₃-C₈ carbocyclo-, —O—(C₁ C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀ heteroalkylene-C(═O)—, —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈carbocyclo-S—, —O—, (C₁-C₈ alkyl)-)—S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, —(C₃-C₈ heterocyclo)-C₁-C₁₀ alkylene-S—, —C₁-C₁₀ alkylene-O—C(═O)—, —C₃-C₈ carbocyclo-O—C(═O)—, —O—(C₁-C₈ alkyl)—O— C(═O)—, -arylene-O—C(═O)—, —C₁-C₁₀ alkylene-arylene-O—C(═O)—, -arylene-C₁-C₁₀ alkylene-O— C(═O)—, —C₁-C₁₀ alkylene-(C₃-C₈carbocyclo)-O—C(═O)—, —(C₃-C₈ carbocyclo)-C₁-C₁₀ alkylene-O—C(═O)—, —C₃-C₈ heterocyclo-O—C(═O)—, —C₁-C₁₀ alkylene-(C₃-C₈heterocyclo)-O—C(═O)—, —(C₃-C₈ heterocyclo)-C₁-C₁₀ alkylene-O—C(═O)—,
any of the R₆ group is optionally substituted with one or more of the substituents selected from: —X, —R′, —O⁻, —OR′, ═O, —SR′, —S⁻, —NR′₂, —NR′₃⁺, ═NR′, —CX₃, —CN, —OCN, —SCN, —N═C═O, —NCS, —NO, —NO₂, ═N₂, —N₃, —NR′C(═O)R′, —C(═O)R′, —C(═O)NR′₂, —SO₃⁻, —SO₃H, —S(═O)₂R′, —OS(═O)₂OR′, —S(═O)₂NR′, —S(═O)R′, —OP(═O)(OR′)₂, —P(═O)(OR′)₂, —PO₃⁻, —PO₃H₂, —C(═O)X, —C(═S)R′, —CO₂R′, —CO₂, —C(═S)OR′, C(═O)SR′, C(═S)SR′, C(═O)NR′₂, C(═S)NR′₂, and C(═NR′)NR′₂, where each X is independently a halogen: —F, —CI, —Br, or —I; and each R′ is independently —H, —C₁C₂₀ alkyl, —C₆-C₂₀ aryl, or —C₃-C₁₄ heterocycle,
Z is an optional spacer,
L is an orthogonal connector,
X is an optional cleavable moiety for releasing D,
D is a cytotoxic drug,
a is 1 or more and b is 0, 1 or more, and
HP_{SMW} results from covalent binding to said orthogonal connector L, of a single molecular weight homopolymer having formula (I) 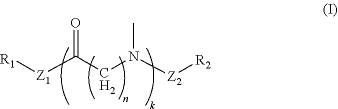
wherein
R₁ and R₂ are different, and
one of R₁ and R₂ is H or an inert group, the other one of R₁ and R₂ being a functionalized reactive group, said group being reactive for covalently binding a bindable group, in such reaction conditions that the inert group is non-reactive,
Z₁ and Z₂, identical or different, are optional spacers, and
n is 1 or more and k is 2 or more.

17. The compound of claim 16, wherein said single molecular weight homopolymer is polysarcosine.
